(19) United States
(12) Patent Application Publication
(10) Pub. No.: US 2008/0193457 A1
(43) Pub. Date: Aug. 14, 2008

(54) Ra Antigenic Peptides

(76) Inventors: Nikolaos Berntenis, St. Louis (FR); Gerrit Buurman, Weil am Rhein (DE); Harald Kropshofer, Loerrach (DE); Bernd Christian Mueller, Weil am Rhein (DE); Sebastian Thomas Spindeldreher, Loerrach (DE); Anne Vogt, Loerrach (DE); Werner Zolg, Weilheim (DE)

Correspondence Address: HOFFMANN-LA ROCHE INC.;PATENT LAW DEPARTMENT, 340 KINGSLAND STREET, NUTLEY, NJ, 07110, US

(21) Appl. No.: 10/567,386

(22) PCT Filed: Jul. 30, 2004
(86) PCT No.: PCT/EP04/08609

§ 371 Date: Apr. 1, 2008

(30) Foreign Application Priority Data
Aug. 7, 2003 (EP) 03017551.7

(51) Int. Cl. A61K 39/395; C07K 7/00; C07K 14/00; C07K 16/18; C12N 15/11; A61P 43/00; C12N 15/00; C12N 5/06; A61K 38/00; G01N 33/00

(52) U.S. Cl. 424/139.1; 530/328; 530/350; 530/387.9; 536/23.5; 435/320.1; 435/325; 514/16; 514/12; 436/86

(57) ABSTRACT

The present invention provides novel naturally-processed MHC class II antigenic peptides; which originate from interferon-γ-inducible lysosomal thiol reductase, integrin beta-2, phosphatitylinositol-4,5-bisphosphate 3-kinase, urokinase-type plas-minogen activator, immunoglobulin heavy chain V-III region (V_{H}26), DJ-1 protein, apolipoprotein B-100, 26S proteasome non-AT-Pase regulatory subunit 8, interleukin-1 receptor, fibromodulin, GM-CSF/IL-3/IL-5 receptor, sorting nexin 3, inter-α-trypsin inhibitor heavy chain H4, complement C4, complement C3 (α-chain), complement C3 (β-chain), SH3 domain-binding glutamic acid-rich-like protein 3, interleukin-4-induced protein 1, hemopexin, Hsc70-interacting protein, invariant chain (Ii), retinoic acid receptor responder protein 2, fibronectin, cathepsin B, tripeptidyl-peptidase II, legumain, platelet activating factor receptor, poly-alpha-2.8-sialyltrans-ferase, and ras-leated protein Rab-11B. Also provided are these antigenic peptides and the proteins they are derived from as markers for erosive and/or non-erosive RA. Moreover, these antigenic peptides linked to MHC class II molecules, antibodies reactive with said antigenic peptides, nucleic acids encoding said antigenic peptides, and nucleic acid constructs, host cells and methods for expressing said antigenic peptides are provided. The antigenic peptides of the invention can be used as markers in diagnosis of RA and in therapy as anti-RA vaccines.

The present invention provides novel naturally-processed RA antigenic peptides which are candidate markers for erosive and non-erosive RA. These antigenic peptides are presented by human MHC class II HLA-DR molecules. Moreover, these antigenic peptides linked to MHC class II molecules, as well as antibodies reactive with said antigenic peptides, nucleic acids encoding said antigenic peptides, and nucleic acid constructs and host cells for expressing said antigenic peptides are provided. The antigenic peptides of the invention as well as the polypeptides they are derived from can be used as markers in diagnosis of RA and in therapy as anti-RA vaccines.

Rheumatoid Arthritis (RA), originally termed chronic polyarthritis, is a systemic autoimmune disease and one of the most debilitating forms of articular inflammation (Feldmann, M. et al., Cell 85 (1996) 307-310; Dedhia, H. V. & DiBartolomeo, A., Critical care clinics 18 (2002) 841-854). Typically, RA causes joint pain, deformities and severe joint stiffness. The disease can also have its manifestation outside the joints, especially in patients who are positive for an autoantibody, termed “rheumatoid factor” (RF) (Mageed, R. A., in: van Venrooij, W. J. & Maini, R. N. eds., Manual of biological markers of disease, Kluwer Academic Publishers (1996) 1-18). RA occurs quite frequently in the Caucasian population with the susceptibility to RA being influenced by genetic and environmental factors. Both have a crucial effect on the onset and the progression of this autoimmune disease. Approximately 4% of the total population has an increased genetic susceptibility to RA, roughly 20% of which (around 1% of the total population) develops RA as a result of, as yet, uncharacterized non-inheritable factors. Beyond that, RA shows a significant bias in the sex ratio: women have a three fold higher risk for RA than men, indicating that sex hormones may also be involved in the pathogenesis.

In the beginning, RA progresses slowly. Typical early stage symptoms are palm sweating, morning stiffness of fingers and symmetrical joint inflammation. In addition, rheumatoid nodules can appear which is an indication for tissue affection outside the joints. In a simplified model, the immune system produces autoantibodies against healthy tissue. These autoantibodies attack the articular cartilage in the joint leading to its inflammation and later on to its destruction. This destruction stimulates the immune system to produce more autoantibodies. In addition, cytokines like tumor necrosis-factor alpha (TNF-α) and Interleukin-1 (IL-1) are produced which enhance the inflammatory reaction even further (Houssiau, F. A., Clin Rheumatol 14 Suppl 2 (1995) 10-13). The synovium begins to swell due to infiltration of additional cells of the immune system, such as macrophages and T cells. These cells are actively involved in causing further cell death and in driving joint inflammation (Fox, D. A., Arthritis Rheum 40 (1997) 598-609; Choy, E. H. & Panayi, G. S., N Engl J Med 344 (2001) 907-916). This process resembles a vicious circle of autoantibody production, joint inflammation and joint destruction.

Typically, RA progresses chronically, with 85-90% of all RA patients showing a mild to moderate disease development. Aggressive disease forms leading to complete loss of joint function up to the degree of invalidity is experienced by 10-15% of the patients. In this advanced RA state, patients have a permanent articular inflammation and display rheumatoid nodules. They suffer from strong chronical pain and the inflammation leads to severe finger stiffness and irreversible joint deformations or dislocations.

Diagnosis

There is growing evidence that therapeutic intervention early in the disease can reduce the extent of joint damage (Egsmose, C. et al., J Rheumatol 22 (1995) 2208-2213; Van der Heide, A. et al., Ann Intern Med 124 (1996) 699-707). Since treatment with disease-modifying antirheumatic drugs (DMARDs) is only justified when the risk:benefit or cost:effectiveness ratios are favorable, it is mandatory to be able to differentiate between RA and other forms of arthritis shortly after onset of the disease (Kirwan, J. R. & Quilty, B., Clin Exp Rheumatol 15 (1997) 15-25). The diagnosis is made by established criteria based on clinical history, physical examination and laboratory tests. The American Society of Rheumatism published a catalog of criteria to help gaining objective evidence for RA (Arnett, P. C. et al., Arthritis Rheum 31 (1987) 315-324). But so far, not a single test is available which is specific for RA. Several biological and biochemical markers, e.g. C-reactive protein (CRP), erythrocyte sedimentation rate (ESR), antinuclear antibody (ANA) or RF are utilized for the evaluation of RA. However, these markers are non-specific, as they appear in other inflammatory or autoimmune diseases as well. The RF, for instance, is an autoantibody that is present in the serum of approximately 50% of RA patients. Since increased levels of the same autoantibody can also be found in the context of other inflammatory diseases, such as Sjögren syndrome, endokarditis or chronical hepatitis, RF is unsuitable to serve as a diagnostic marker for RA. Rather than being of diagnostic value per se, the above mentioned biochemical and biological markers are useful for assessing disease activity and prognosis as well as in the treatment and management of RA patients (Nakamura, R. M., J Clin Lab Anal 14 (2000) 305-313).

Recently, a diagnostic set of criteria was developed that consists of clinical and biochemical aspects which were claimed to discriminate, at an early state, between self-limiting, persistent non-erosive, and persistent erosive RA (Visser, H. et al., Arthritis Rheum 46 (2002) 357-365). Self-limiting arthritis was characterized by natural remission: there was no arthritis on examination in a patient for a certain period of time. Erosive arthritis was defined based on the presence of erosions on radiographs of the hands and/or feet. In particular, the use of antibodies recognizing cyclic citrullinated peptides appears to be promising and suggests an important role for citrullinated antigens in the early diagnosis and prognosis of erosive RA (Schellekens, G. A. et al., J Clin Invest 101 (1998) 273-281; Vincent, C. et al., J Rheumatol 25 (1998) 838-846). The early recognition of erosive RA allows early intervention with DMARDs, which will lead to earlier disease control and improvement of disease outcome (Symmons, D. P. M. et al., J Rheumatol 25 (1998) 1072-1077; Anderson, J. J. et al., Arthritis Rheum 43 (2000) 22-29). Likewise, early recognition of self-limiting and non-erosive arthritis will prevent unnecessary treatment with potentially toxic therapeutics (Fries, J. F. et al., Arthritis Rheum 36 (1993) 297-306.

Therapy

The goal of any anti-rheumatic therapy is to relieve pain in order to ease the activities of every day life. So far, complete healing of RA is not possible, but by applying modern therapies the progression of the disease can be slowed down or even stopped. Due to individual differences, each patient requires an individualized therapy and early diagnosis, as mentioned before, is desirable. RA therapy is complex and includes lifelong medicinal treatment as well as physio- and radiotherapy. DMARDs used in RA therapy are basic therapeutics (e.g. Methotrexate, Sulfasalazin, Hydroxychloroquin, Leflunomid, Azathioprin), cortisone, non-steroidal anti-inflammatory drugs (NSAID) or monoclonal antibodies against the pro-inflammatory cytokines TNF-α, IL-1β or their respective receptors (http://rheuma-online.de). These drugs have all in common that they are inhibitors of inflammation by suppressing the immune response. The main disadvantage is their lack of specificity for RA, their adverse effects and their inability to effectively target the causes of RA.

Autoimmunity

Autoimmunity starts when a specific adaptive immune response is initiated against self antigens (autoantigens) manifested by the development of self-reactive T or B cells. The normal consequence of an adaptive immune response against a foreign antigen is the clearance of the antigen from the body. When an adaptive immune response develops against a self antigen, however, the antigen can in most cases not be completely removed from the body, leading to a sustained immune response. As a consequence, the effector mechanisms of immunity cause chronic inflammatory injury to tissues. The mechanisms of tissue damage are essentially the same in autoimmune disease as those that operate in protective immunity and in hypersensitivity. Even though it is not well understood what triggers autoimmunity, several events which are nowadays believed to contribute to the induction of autoimmune diseases and selection of autoantigenic targets have been summarized most recently (Marrack, P. et al., Nat Med 7 (2001) 899-905).

Autoimmune diseases are controlled by properties of particular genes of each individual and environmental factor. The host's genes affect the susceptibility to autoimmunity at least at three levels. First, some of the genes affect the overall reactivity of the immune system and, thus, can predispose the individual to certain or to several different types of autoimmune diseases. Second, this altered immunoreactivity is funneled to particular autoantigens and tissues by other genes that affect recognition of antigenic peptides by T cells. Third, still other genes act on the ability of target tissues to modulate immune attack for instance by influencing the activity of effector cells of the immune system which are destined to initiate an autoaggressive attack. The latter two sets of genes dictate which antigens will be the targets of autoimmunity and hence which organs will be attacked and what damage will occur.

In addition, signals from the environment influence the development of autoimmunity at the same three levels, by affecting the overall reactivity of the immune system, the antigen-specificity and the state of the potential target tissue. And finally, there is cross-talk between genetic and environmental factors.

Major Histocompatibility Complex (MHC)

Population studies, genotyping and modern approaches at the molecular level have unanimously shown that certain genes encoded by the major histocompatibility complex (MHC) confer a significantly higher risk for the development of RA (Stastny, P., Tissue Antigens 4 (1974) 571-579; Wordsworth, P. et al., PNAS 86 (1989) 10049-10053; Wordsworth, P. & Bell, J., Springer Semin Immunopathol 14 (1992) 59-78). In particular, the class II MHC alleles HLA-DRB1*0101, *0401, *0404 and *0405 in several ethnic groups increase the susceptibility to RA (Reveille, J., Curr Opin Rheumatol 10 (1998) 187-200). E.g. more than 90% of RF-positive RA patients carry one of these susceptibility alleles. HLA class II molecules are MHC-surface proteins that bind antigenic peptides within the cell and present them on the surface of antigen-presenting cells for interaction with the T cell receptors of CD4⁺ helper T lymphocytes, thereby initiating a cellular immune response (Banchereau, J. & Steinman, R. M., Nature 392 (1998) 245-252). The RA-association of particular HLA class II molecules together with the presence of large numbers of activated CD4⁺ T cells in synovial tissue has supported the model of disease induction in which disease-associated HLA-DR molecules present disease-relevant (e.g. synovial) autoantigens and cause stimulation and expansion of synovial T cells, which then drive the inflammatory process (Striebich, C. C. et al., J Immunol 161 (1998) 4428-4436).

MHC class II HLA-DR (short: DR) proteins are heterodimers consisting of monomorphic α- and extremely polymorphic β-chains that bind peptide antigens in a peptide binding groove. This groove generally has four major pockets to accept side chains at relative positions 1, 4, 6 and 9 of the peptide (Stern, L. J. et al., Nature 368 (1994) 215-221). The allelic variations between HLA class II molecules account for the differential ability to bind antigenic peptides. This is the rationale why individuals differing in their HLA alleles have divergent antigenic peptide repertoirs, thereby leading to differences in the quality of immune responses (Messaoudi, I. et al., Science 298 (2002) 1797-1800).

Peptides bound by class II MHC molecules are typically longer and more heterogeneous in size (11-25 amino acids) than the peptides bound by class I MHC molecules (8-10 amino acids). This difference arises because the peptide binding groove of class II proteins is open and while peptides are gripped in the middle, their ends can extend out of the groove in a variable fashion (Jones, E. Y., Curr Opin Immunol 9 (1997) 75-79). As a consequence, class II molecules typically bind sets of overlapping peptides that share a common core sequence, termed “T cell epitope”, but have different lengths.

More than a decade ago, it was recognized that the DRβchains encoded by RA-linked DRB1 alleles, although exhibiting polymorphic differences, all share a stretch of identical or almost identical amino acids at positions 67-74, known as the “shared epitope” (Gregersen, P. K. et al., Arthritis Rheum 30 (1987) 1205-1213). Since immunity to autoantigens has been regarded central to the pathogenesis of RA, it was hypothesized that the shared epitope could impose disease linkage on the respective DR molecules by at least two different mechanisms: first, by selecting the relevant autoantigenic peptides for presentation, and second, by selecting the appropriate autoreactive T cell specificities during ontogeny. The three-dimensional structure of DR molecules has indeed revealed that the shared epitope is located in the center of the α-helix flanking one side of the peptide binding groove (Stern, L. J. et al., Nature 368 (1994) 215-221). Thus, strategically this shared epitope region is positioned in such a way that it can interact with both bound peptide and T cell receptor.

However, one of the unresolved mysteries in rheumatology research is the question what are the key arthritogenic antigens and epitopes in man that trigger the onset and the development of RA. Although autoantibodies of different specificity have been identified in serum and synovial fluid of patients it is often unclear whether the antigens which were released at the time of cartilage degradation, were initiating pathogenicity or whether they are merely a consequence of antigen spreading as a result of inflammation (Corrigall, V. M. & Panayi G. S., Crit. Rev Immunol 22 (2002) 281-293). Furthermore it is difficult to define pathogenic mechanisms in which the antigen is present throughout the body, including the joint, but the pathology is targeted solely or predominately to the joint.

Autoantigens

The large number of possible autoantigens in RA is derived from studies using sera or, less frequently, T cells from patients with established chronic RA. One of the most convincing joint-specific antigen that has been proposed in the context of DR molecules, is type II collagen (CII), the predominant protein in articular cartilage. Autoantibodies against CII were found in elevated concentrations in the serum and joints of RA patients although it is not yet clear whether anti-CII antibodies are pathogenic in RA (Banerjee, S. et al., Clin Exp Rheumatol 6 (373-380). Snowden and coworkers have shown that peripheral blood T cells from RA patients proliferated to CII, most pronounced in those patients with anti-CII antibodies. However, the response was seen only in 50% of patients (Snowden, N. et al., Rheumatology 40 (1997) 1210-1218). In a mouse model immunization with CII was shown to induce arthritis in mice expressing the class II MHC alleles DRB1*0401 and *0101 (Rosloniec, E. F. et al., J Exp Med 185 (1997) 1113-1122; Rosloniec, E. F. et al., J immunol 160 (1998) 2573-2578). The immunodominant epitope in both *0401 and *0101 transgenic mice was localized to peptides within residues 261-273 of human CII (Fugger, L. et al., Eur J Immunol 26 (1996) 928-933). The same epitope of CII was capable of stimulating a T cell response in RA patients, particularly in the early stages of disease. Synovial fluid T cells were especially responsive (Kim, H. Y. et al., Arthritis Rheum 42 (1999) 2085-2093).

Although other cartilage proteins have been proposed as RA candidate antigens, DR4-binding epitopes have been defined only for human cartilage glycoprotein 39 (HCgp39). This protein is secreted by synovial cells and articular chondrocytes and its expression is upregulated in plasma and joints during inflammation (Vos, K. et al., Ann Rheum Dis 59 (2000) 544-548). Similar to CII, HCgp39 treatment induces arthritis in mice. In addition a HCgp39 response of peripheral blood T cells from RA patients was detected (Verheijden, G. F. et al., Arthritis Rheum 40 (1997) 1115-1125). The predominant epitope recognized by T cells in DR4 patients was defined between residues 263-275 and identical to the immunodominant epitope found in DRB1*0401-transgenic mice after immunization with native HCgp39 (Cope, A. P. et al., Arthritis Rheum 42 (1999) 1497-1507). Although not disease specific, responses to this peptide did correlate with disease activity in RA patients (Baeten, D. et al., Arthritis Rheum 43 (2000) 1233-1243). Antibodies to HCgp39, however, have also been detected in the sera of patients with inflammatory diseases, such as inflammatory bowel disease and systemic lupus erythiematosus (SLE), albeit at a lower level than in RA.

In an attempt to track antigen-specific T cells in RA, soluble peptide-DR4 tetrameric complexes were used to detect synovial CD4⁺ T cells reactive with CII or HCgp39 in DR4⁺ patients (Kotzin, B. L. et al., PNAS 97 (2000) 291-296). The CII-DR4 complex bound in a specific manner to CII peptide-reactive T cell hybridomas, but did not stain a detectable fraction of synovial CD4⁺ cells. Almost similar results were obtained with the HCgp39-DR4 complex suggesting that the major oligoclonal CD4⁺ T cell expansions present in RA joints are not specific for the dominant CII and HCgp39 determinants described above.

In summary, despite some strong indications for a CII and HCgp39 association with RA, the evidence that they are important antigens in RA is scanty. A direct proof that peptides of CII or HCgp39 are presented in a class II MHC-restricted manner by antigen-presenting cells with subsequent stimulation and activation of synovial CD4⁺ T cells is still lacking. Furthermore a major problem of animal models is their unknown relevance to RA as CII-induced arthritis by immunizing rats or mice differs in many respects from RA.

Naturally Processed MHC Class II-Associated Peptides

An alternative strategy to the identification of RA-specific autoantibodies and T cells relies on the sequence analysis of naturally processed peptide antigens bound to MHC class II molecules. With the help of monoclonal antibodies, class II MHC molecules conferring susceptibility to RA can be purified from cognate cells. RA-associated peptide antigens can be acid-eluted from purified HLA class II molecules. The mixture of small peptides can be separated by HPLC and the peptide sequence be determined by Edman sequencing or mass spectrometry. Due to limitations with peptide purification and sequencing techniques, peptide sequences were, as yet, only obtained from MHC molecules that have been isolated from cultured B cell lines or large amounts of tissue, and the analysis was restricted to a few abundant peptides (Kropshofer et al., J. Exp. Med. 175 (1992) 1799-1803; Chicz, R. M. et al., J Exp Med 178 (1993) 27-47). As a result of the development of high-resolution microcapillary HPLC columns and more sensitive mass spectrometers, MHC-bound peptides can be analyzed more efficiently (Dongre, A. R. et al., Eur J Immunol 31 (2001) 1485-1494; Engelhard, V. H. et al., Mol Immunol 39 (2002) 127-137).

In the present invention a modified peptide isolation and sequencing technique was used to investigate the peptide antigen repertoire of HLA-DR4 molecules derived from autologous dendritic cells (DCs) which were pulsed with serum or synovial fluid derived from RA patients. The main advantage of this innovative approach is the usage of human DCs that are professionals in RA-relevant antigen processing and presentation, instead of using transgenic animal models or artificial B cell lines.

DCs are enriched in rheumatoid synovial fluid and tissue and are derived from circulating immature precursors (Thomas, R. et al., J Immunol 152 (1994) 2613-2623). They are the most potent antigen-presenting cells which express high levels of MHC molecules together with a variety of accessory molecules (Mellman, I. et al., Trends Cell Biol 8 (1998) 231-237). In a most recent study, it was shown that ex vivo differentiated human DCs and macrophages that are phenotypically similar to antigen-presenting cells from RA synovial joints, were capable of generating and presenting immunodominant epitopes from CII and HCgp39 (Tsark, E. C. et al., J Immunol 169 (2002) 6625-6633). DC have the capacity to prime CD4⁺ helper T cells and to effectively activate cytotoxic CD8⁺ T cells (Ridge, T. et al., Nature 393 (1998) 474-478). Thus, peptides bound to MHC class II molecules and presented by DCs play a superior role in the pathogenesis of diseases involving T cell-driven immune responses.

Therefore, the problem posed by the lack of knowledge of MHC class II restricted antigenic peptides for RA is solved by providing novel naturally-processed MHC class II associated RA antigenic peptides and the polypeptides they are derived from as markers for RA.

The present invention provides novel naturally-processed antigenic peptides which are candidate RA markers in erosive and non-erosive RA. These antigenic peptides are presented by human MHC class II HLA-DR molecules derived from dendritic cells which were pulsed with serum or synovial fluid derived from patients with established erosive or non-erosive RA. The MHC class II antigenic peptide of the invention are comprising (a) at least the amino acid sequence of the peptide binding motif selected from the group consisting of SEQ ID NOs. 49 to 57 and SEQ ID NOs. 103 to 122, or (b) at least the amino acid sequence of the peptide binding motif selected from the group consisting of SEQ ID NOs. 49 to 57 and SEQ ID NOs. 103 to 122 with additional N- and C-terminal flanking sequences of a corresponding sequence selected from the group consisting of SEQ ID NOs. 1 to 39 or SEQ ID NOs. 58 to 102, and originate from interferon-γ-inducible lysosomal thiol reductase, integrin beta-2, phosphatitylinositol-4,5-bisphosphate 3-kinase, urokinase-type plasminogen activator, immunoglobulin heavy chain V-III region (V_{H}26), DJ-1 protein, apolipoprotein B-100, 26S proteasome non-ATPase regulatory subunit 8, interleukin-1 receptor, fibromodulin, GM-CSF/IL-3/IL-5 receptor, sorting nexin 3, inter-α-trypsin inhibitor heavy chain H4, complement C4, complement C3 (α-chain), complement C3 (β-chain), SH3 domain-binding glutamic acid-rich-like protein 3, interleukin-4-induced protein 1, hemopexin, Hsc70-interacting protein, invariant chain (Ii), retinoic acid receptor responder protein 2, fibronectin, cathepsin B, tripeptidyl-peptidase II, legumain, platelet activating factor receptor, poly-alpha-2.8-sialyltransferase, and ras-leated protein Rab-11B. The present invention also provides these antigenic peptides and the proteins they are derived from as markers for erosive and/or non-erosive RA. Moreover, these antigenic peptides linked to MHC class II molecules, as well as antibodies reactive with said antigenic peptides, nucleic acids encoding said antigenic peptides, and nucleic acid constructs, host cells and methods for expressing said antigenic peptides are provided. Further methods are provided for isolating and identifying RA antigenic peptides.

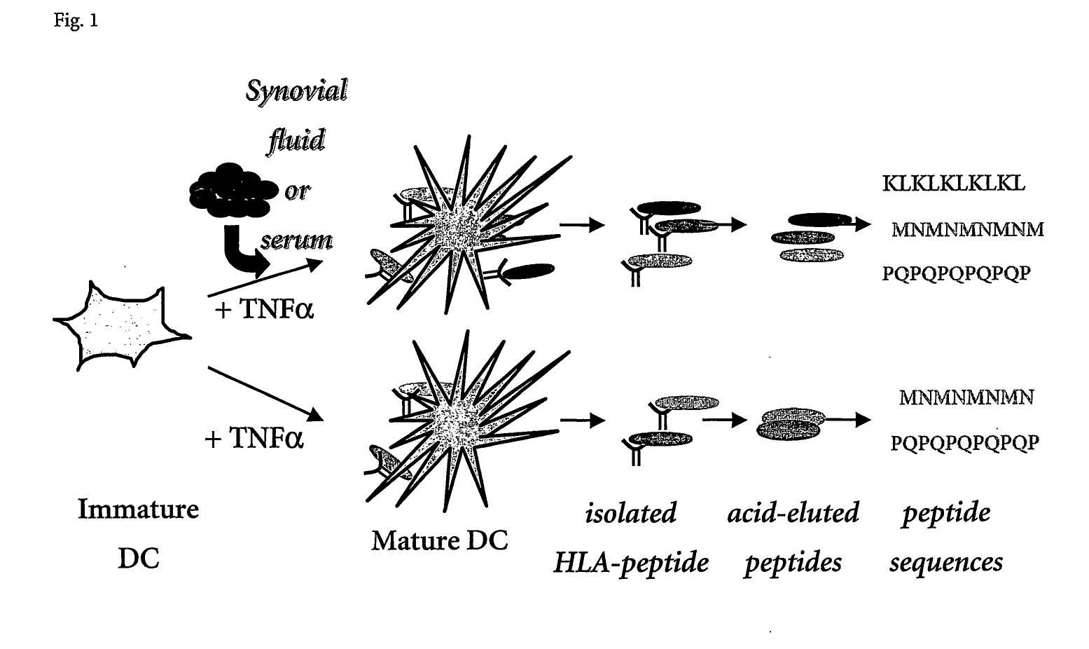

FIG. 1: Diagram of Dendritic cell (DC)-mediated analysis of tissue samples: Dendritic cells (DCs), the most specialized antigen-presenting cells (APCs), are brought in contact with an antigen source (e.g. synovial fluid) under optimal conditions for antigen uptake and antigen processing. As a control, DCs are cultured under the same conditions in the absence of synovial fluid antigens. After maturation of DCs, antigen-loaded MHC class II molecules are purified and the respective MHC class II-associated antigenic peptides are isolated and identified.

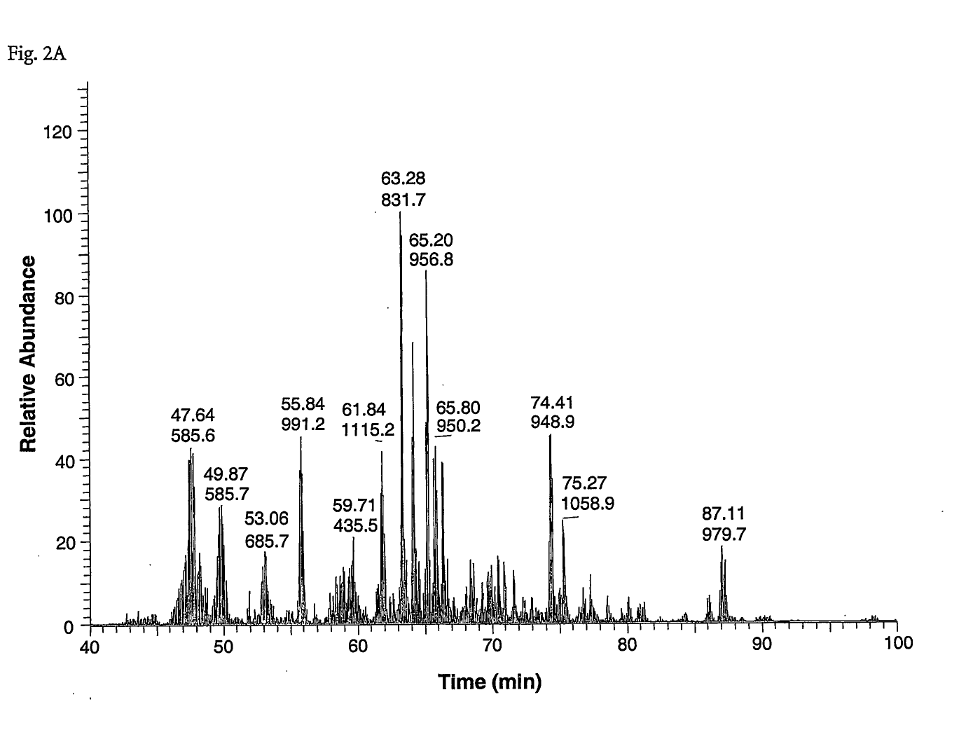

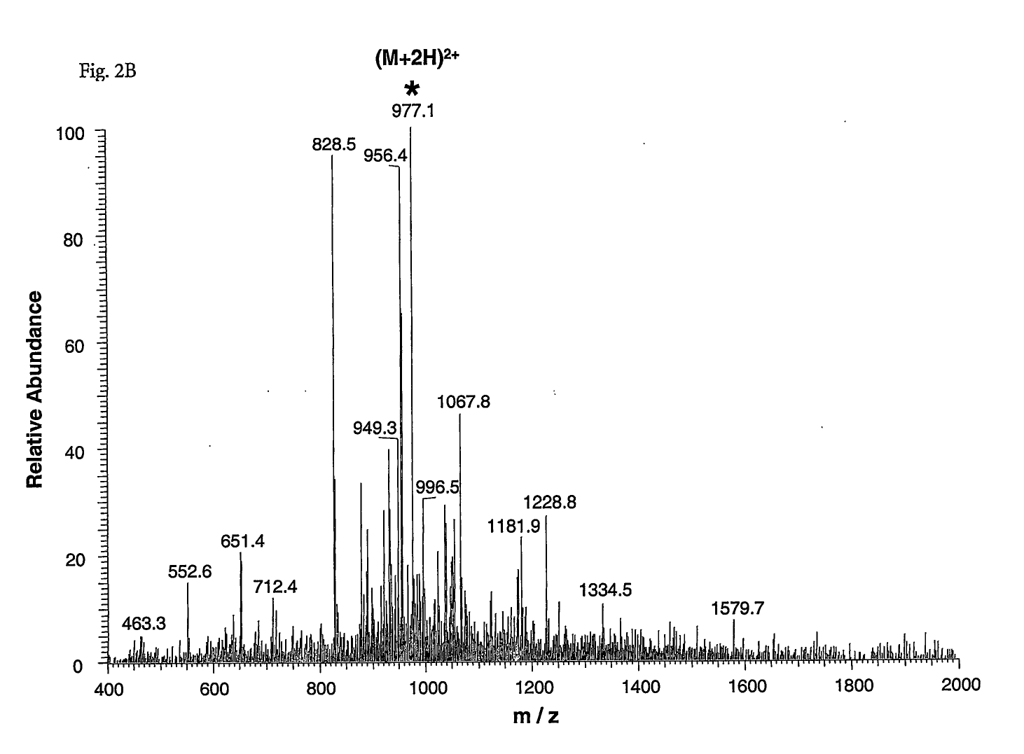

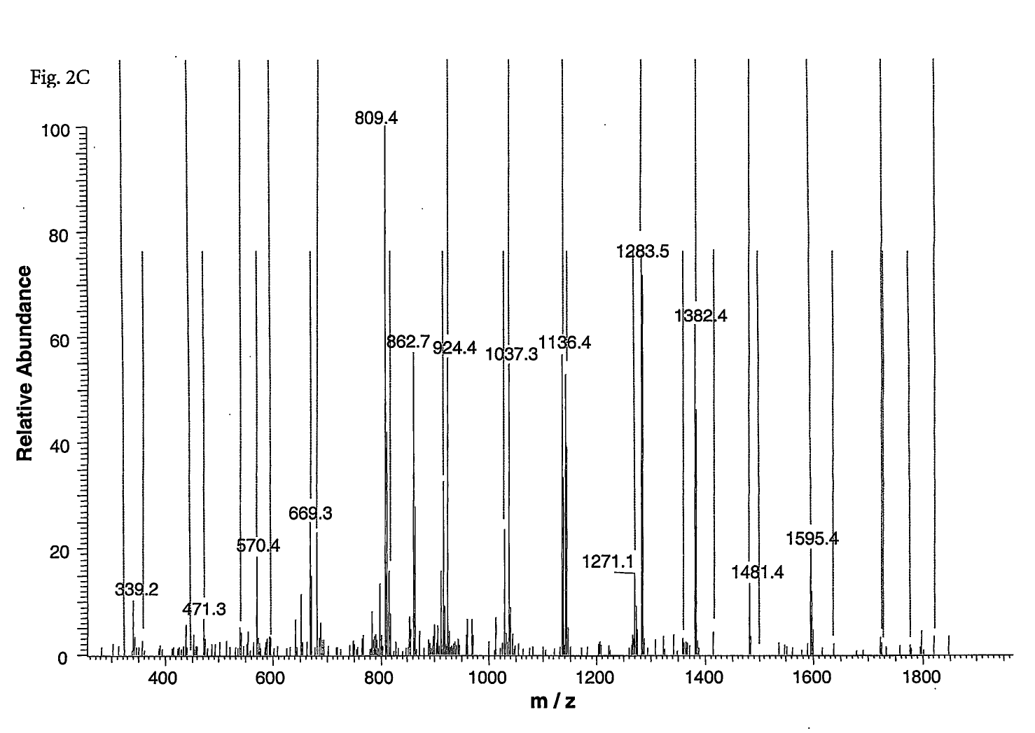

FIG. 2A: ION-TRAP MS Base Peak Chromatogram of MHC class II-associated antigenic peptides that were isolated from dendritic cells pulsed with the serum of a RA patient. The peptides were eluted directly from a RP-C18-HPLC column into the ion trap mass spectrometer for immediate MS/MS identification. The numbers indicate the retention times (upper value) and the molecular masses (lower value) of the most prominent peptide peaks in the mixture at the respective time.

FIG. 2B: ION-TRAP MS spectrum of antigenic peptides at a retention time of 65.4 min. The marked peak was further fragmented and corresponded to a doubly charged peptide ion from the inter-alpha-trypsin inhibitor ITIH4 (cf. table 3).

FIG. 2C: ION-TRAP MS/MS spectrum of the doubly charged peptide ion at m/z 977.1. The fragmentation masses, together with the mass of the parent ion, were searched against a non-redundant human database by using the SEQUEST algorithm. The retrieved sequence MPKNVVFVIDKSGSMSGR (one-letter-code) corresponded to the dominant epitope ITIH4 (271-288) of the inter-alpha-trypsin inhibitor. The positions of the assigned series of N-terminal B-ions and C-terminal Y-ions are marked.

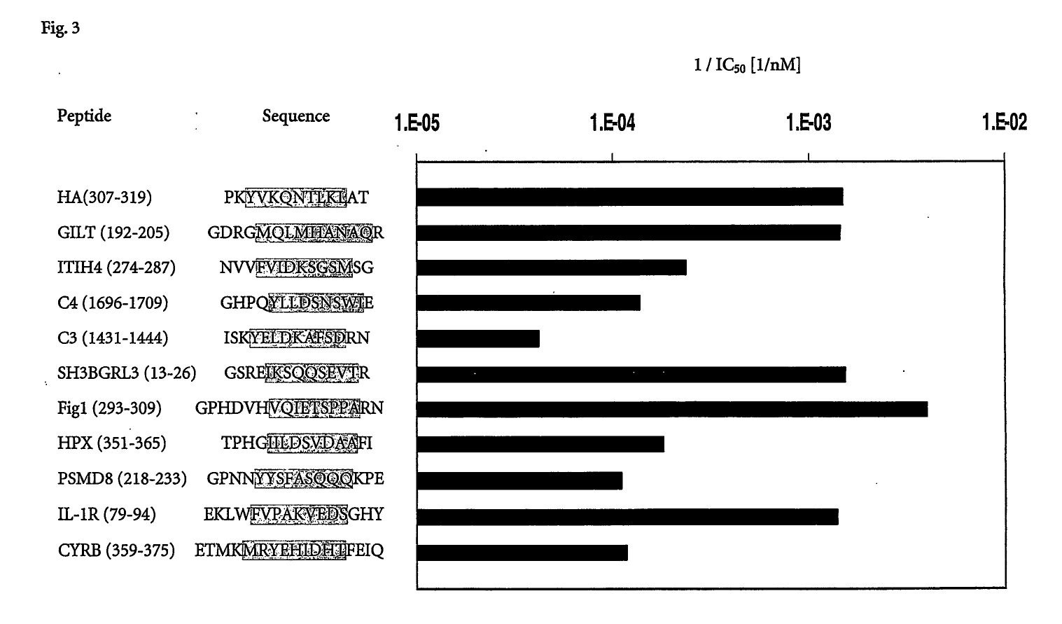

FIG. 3: Summary of the differential binding capacity of the tested candidate RA antigens in the context of binding to the HLA-DRB1*0401 allele. The putative HLA-DRB1*0401 binding motif is boxed in grey. and. As a measure for affinity, the peptide concentration was determined that was needed to reduce binding of a fixed amount of biotinylated HA(307-319) peptide by 50% (IC₅₀) through competition. The reciprocal (1/IC₅₀) directly correlates with peptide affinity. As a reporter biotinylated HA(307-319) peptide from influenza hemagglutinin (Rothbard, J. B. et al., Cell 52 (1988) 515-523) was included in the study.

The antigenic peptides of the invention are peptides, which are associated with and presented by MHC molecules and thereby can have the potential to activate or tolerize T cells. Antigenic peptides presented by MHC class II molecules are therefore MHC class II associated or MHC class II antigenic peptides, whereas antigenic peptides presented by MHC class I molecules are MHC class I associated or MHC class I antigenic peptides.

Peptides which are derived from proteins that are encoded in the genome of the body or an APC are denoted as “self-peptides”. The main function of self-peptides presented by DCs in the peripheral lymphoid organs is thought to be the induction of T cell tolerance to self-proteins. Tolerance is the failure to respond to an antigen; when that antigen is borne by self tissues, tolerance is called self tolerance.

Antigens which are derived from an individual's own body are called “self antigens” or “autoantigens”. An adaptive immune response directed against self antigens is called an autoimmune response. Likewise, adaptive immunity specific for self antigens is called autoimmunity. Autoreactivity describes immune responses directed against self antigens. RA is probably due to an autoimmune response that is based on the involvement of autoreactive T cells and/or autoreactive antibodies. Immunogenic peptide includes, but is not limited to, an antigenic peptide capable of causing or stimulating a cellular or humoral immune response. Such peptides may also be reactive with antibodies.

Peptides derived from proteins encoded in the genome of bacteria, viruses or other foreign invaders and which differ from self-proteins are called “foreign antigenic” or “foreign” peptides. They are able to elicit a T cell response against foreign proteins they are derived from.

RA antigenic peptides are self-peptides that function as self antigens and as a consequence of the disease erroneously trigger autoreactivity against self tissues.

The present invention provides a MHC class II antigenic peptide comprising (a) at least the amino acid sequence of the peptide binding motif selected from the group consisting of SEQ ID NOs. 49 to 57 and SEQ ID NOs. 103 to 122, or (b) at least the amino acid sequence of the peptide binding motif selected from the group consisting of SEQ ID NOs. 49 to 57 and SEQ ID NOs. 103 to 122, with additional N- and C-terminal flanking sequences of a corresponding sequence selected from the group consisting of SEQ ID NOs. 1 to 39 and SEQ ID NOs. 58 to 102. Preferably, the MHC class II antigenic peptide has a length of less than 26 amino acids, more preferably a length of 11 to 25 amino acids. Even more preferred is the antigenic peptide of the invention with a length of 11 to 19 amino acids. Most preferred is the antigenic peptide of the invention consisting of the peptide binding motif comprising the four anchor amino acids.

The present invention also provides a MHC class II antigenic peptide comprising (a) at least the amino acid sequence of the peptide binding motif of SEQ ID NO. 49, or (b) at least the amino acid sequence of the peptide binding motif of SEQ ID NO. 49 with additional N- and C-terminal flanking sequences of a corresponding sequence selected from the group consisting of SEQ ID NOs. 1 to 3.

Furthermore, a MHC class II antigenic peptide is provided comprising (a) at least the amino acid sequence of the peptide binding motif of SEQ ID NO. 103, or (b) at least the amino acid sequence of the peptide binding motif of SEQ ID NO. 103 with additional N- and C-terminal flanking sequences of the corresponding sequence of SEQ ID NOs. 58 and 59.

Furthermore, a MHC class II antigenic peptide is provided comprising (a) at least the amino acid sequence of the peptide binding motif of SEQ ID NO. 104, or (b) at least the amino acid sequence of the peptide binding motif of SEQ ID NO. 104 with additional N- and C-terminal flanking sequences of the corresponding sequence of SEQ ID NO. 60.

Furthermore, a MHC class II antigenic peptide is provided comprising (a) at least the amino acid sequence of the peptide binding motif of SEQ ID NO. 105, or (b) at least the amino acid sequence of the peptide binding motif of SEQ ID NO. 105 with additional N- and C-terminal flanking sequences of the corresponding sequence of SEQ ID NO. 61.

Furthermore, a MHC class II antigenic peptide is provided comprising (a) at least the amino acid sequence of the peptide binding motif of SEQ ID NO. 106, or (b) at least the amino acid sequence of the peptide binding motif of SEQ ID NO. 106 with additional N- and C-terminal flanking sequences of the corresponding sequence of SEQ ID NO. 62.

Furthermore, a MHC class II antigenic peptide is provided comprising (a) at least the amino acid sequence of the peptide binding motif of SEQ ID NO. 107, or (b) at least the amino acid sequence of the peptide binding motif of SEQ ID NO. 107 with additional N- and C-terminal flanking sequences of the corresponding sequence of SEQ ID NO. 63.

Furthermore, a MHC class II antigenic peptide is provided comprising (a) at least the amino acid sequence of the peptide binding motif of SEQ ID NO. 50, or (b) at least the amino acid sequence of the peptide binding motif of SEQ ID NO. 50 with additional N- and C-terminal flanking sequences of the corresponding sequence of SEQ ID NO. 5.

Furthermore, a MHC class II antigenic peptide is provided comprising (a) at least the amino acid sequence of the peptide binding motif of SEQ ID NO. 108, or (b) at least the amino acid sequence of the peptide binding motif of SEQ ID NO. 108 with additional N- and C-terminal flanking sequences of the corresponding sequence of SEQ ID NOs. 64 to 67.

Furthermore, a MHC class II antigenic peptide is provided comprising (a) at least the amino acid sequence of the peptide binding motif of SEQ ID NO. 109, or (b) at least the amino acid sequence of the peptide binding motif of SEQ ID NO: 109 with additional N- and C-terminal flanking sequences of the corresponding sequence of SEQ ID NO. 68.

Furthermore, a MHC class II antigenic peptide is provided comprising (a) at least the amino acid sequence of the peptide binding motif of SEQ ID NO: 110, or (b) at least the amino acid sequence of the peptide binding motif of SEQ ID NO:110 with additional N- and C-terminal flanking sequences of the corresponding sequence of SEQ ID NOs. 69 and 70.

Furthermore, a MHC class II antigenic peptide is provided comprising (a) at least the amino acid sequence of the peptide binding motif of SEQ ID NO: 111, or (b) at least the amino acid sequence of the peptide binding motif of SEQ ID NO: 111 with additional N- and C-terminal flanking sequences of the corresponding sequence of SEQ ID NO. 72.

Furthermore, a MHC class II antigenic peptide is provided comprising (a) at least the amino acid sequence of the peptide binding motif of SEQ ID NO. 112, or (b) at least the amino acid sequence of the peptide binding motif of SEQ ID NO. 112 with additional N- and C-terminal flanking sequences of the corresponding sequence of SEQ ID NO. 73.

The MHC class II associated novel antigenic peptides of the invention originate from interferon-γ-inducible lysosomal thiol reductase (SEQ ID NOs. 1 to 3), integrin beta-2 (SEQ ID NOs. 58 and 59), phosphatitylinositol-4,5-bisphosphate 3-kinase (SEQ ID NO: 60), urokinase-type plasminogen activator (SEQ ID NO: 61), immunoglobulin heavy chain V-III region (V_{H}26) (SEQ ID NO: 62), DJ-1 protein (SEQ ID NO: 63), apolipoprotein B-100 (SEQ ID NOs. 4 and 5), 26S proteasome non-ATPase regulatory subunit 8 (SEQ ID NOs. 64 to 67), interleukin-1 receptor (SEQ ID NO: 68), fibromodulin (SEQ ID NOs.: 69 and 70), GM-CSF/IL-3/IL-5 receptor (SEQ ID NOs. 71 and 72), sorting nexin 3 (SEQ ID NO: 73), inter-α-trypsin inhibitor heavy chain H4 (SEQ ID NOs. 6 to 12), complement C4 (SEQ ID NOs. 13 to 18), complement C3 (α-chain) (SEQ ID NOs. 19 to 23, 74 and 75), complement C3 (β-chain) (SEQ ID NOs. 76 and 77), SH3 domain-binding glutamic acid-rich-like protein 3 (SEQ ID NOs. 24 to 27), interleukin-4-induced protein 1 (SEQ ID NOs. 28 to 30), hemopexin (SEQ ID NOs. 31 to 35 and 78), Hsc70-interacting protein (SEQ ID NOs. 36 to 39), invariant chain (II) (SEQ ID NOs. 79 to 83), retinoic acid receptor responder protein 2 (SEQ ID NOs. 84 to 86), fibronectin (SEQ ID NOs. 87 to 91), cathepsin B (SEQ ID NO: 92), tripeptidyl-peptidase II (SEQ ID NOs. 93 and 94), legumain (SEQ ID NO: 95), platelet activating factor receptor (SEQ ID NO: 96), poly-alpha-2.8-sialyltransferase (SEQ ID NO: 97), and ras-leated protein Rab-11B (SEQ ID NOs. 98 to 102).

The single peptide binding groove of MHC class II molecules is about 25 Å long, but in contrast to MHC class I molecules, both sides are open (Stern L J et al., Nature 1994; 368, 215-221). Thus, naturally processed antigenic peptides eluted from human MHC class II molecules have a minimal length of about 11 residues and attain a maximal length of about 25 residues (Chicz R M et al., J Exp Med 1993; 178, 27-47).

The stability of the MHC-peptide interaction is determined by more than a dozen hydrogen bonds involving the peptide backbone and the complementarity between specificity pockets of the binding groove and appropriately located amino acid side-chains of the peptide. The amino acids of the peptide fitting into the respective pockets were named “anchor” residues. With regard to most HLA-DR alleles, these anchors are located at relative positions P1, P4, P6 and P9. The combination of amino acids at these 4 anchor positions conferring high-stability binding to the respective HLA-DR allelic product and vary from allele to allele. The peptide binding motif is defined herein as the sequence of nine amino acids comprising the four anchor amino acids. The peptide binding motif of the MHC class II antigenic peptide of the invention is depicted in SEQ ID NO. 49 for the peptides derived from interferon-γ-inducible lysosomal thiol reductase (SEQ ID NOs. 1 to 3), in SEQ ID NO. 103 for the peptides derived from integrin beta-2 (SEQ ID NOs. 58 and 59), in SEQ ID NO. 104 for the peptides derived from phosphatitylinositol-4,5-bisphosphate 3-kinase (SEQ ID NO: 60), in SEQ ID NO. 105 for the peptides derived from urokinase-type plasminogen activator (SEQ ID NO: 61), in SEQ ID NO. 106 for the peptides derived from immunoglobulin heavy chain V-III region (V_{H}26) (SEQ ID NO: 62), in SEQ ID NO. 107 for the peptides derived from DJ-1 protein (SEQ ID NO: 63), in SEQ ID NO. 50 for the peptides derived from apolipoprotein B-100 (SEQ ID NOs. 4 and 5), in SEQ ID NO. 108 for the peptides derived from 26S proteasome non-ATPase regulatory subunit 8 (SEQ ID NOs. 64 to 67), in SEQ ID NO. 109 for the peptides derived from interleukin-1 receptor (SEQ ID NO: 68), in SEQ ID NO. 110 for the peptides derived from fibromodulin (SEQ ID NOs.: 69 and 70), in SEQ ID NO. 111 for the peptides derived from GM-CSF/IL-3/IL-5 receptor (SEQ ID NOs. 71 and 72), in SEQ ID NO. 112 for the peptides derived from sorting nexin 3 (SEQ ID NO: 73), in SEQ ID NO. 51 for the peptides derived from inter-α-trypsin inhibitor heavy chain H4 (SEQ ID NOs. 6 to 12), in SEQ ID NO. 52 for the peptides derived from complement C4 (SEQ ID NOs. 13 to 18), in SEQ ID NO. 53 for the peptides derived from complement C3 (α-chain) (SEQ ID NOs. 19 to 23, 74 and 75), in SEQ ID NO. 113 for the peptides derived from complement C3 (i-chain) (SEQ ID NOs. 76 and 77), in SEQ ID NO. 54 for the peptides derived from SH3 domain-binding glutamic acid-rich-like protein 3 (SEQ ID NOs. 24 to 27), in SEQ ID NO. 55 for the peptides derived from interleukin-4-induced protein 1 (SEQ ID NOs. 28 to 30), in SEQ ID NO. 56 for the peptides derived from hemopexin (SEQ ID NOs. 31 to 35 and 78), in SEQ ID NO. 57 for the peptides derived from Hsc70-interacting protein (SEQ ID NOs. 36 to 39), in SEQ ID NO: 114 for the peptides derived from invariant chain (Ii) (SEQ ID NOs. 79 to 83), in SEQ ID NO. 115 for the peptides derived from retinoic acid receptor responder protein 2 (SEQ ID NOs. 84 to 86), in SEQ ID NO. 116 for the peptides derived from fibronectin (SEQ ID NOs. 87 to 91), in SEQ ID NO. 117 for the peptides derived from cathepsin B (SEQ ID NO: 92), in SEQ ID NO. 118 for the peptides derived from tripeptidyl-peptidase II (SEQ ID NOs.93 and 94), in SEQ ID NO. 119 for the peptides derived from legumain (SEQ ID NO: 95), in SEQ ID NO. 120 for the peptides derived from platelet activating factor receptor (SEQ ID NO: 96), in SEQ ID NO. 121 for the peptides derived from poly-alpha-2.8-sialyltransferase (SEQ ID NO: 97) and in SEQ ID NO. 122 for the peptides derived from Ras-related protein Rab-11B (SEQ ID NO: 98 to 102).

The peptide binding motif may also comprise at least one, at least two, at least three, at least four or at least five modifications of the amino acid sequence while still attaining the binding capacity of the non-modified peptide binding motif. Preferably, the modified peptide binding motif comprises at least three of the four anchor amino acids of the non-modified peptide binding motif. The amino acid modification may be a conservative amino acid substitution as described below.

Additional binding energy is provided by hydrogen bonds involving residues in front of the P1 anchor and behind the P9 anchor. In agreement with that, in most naturally processed peptides the nonameric core-region (P1-P9) is N- and C-terminally flanked by 3-4 residues. Hence, the majority of peptides are 15-17-mers. Longer peptides protrude from the groove, thereby allowing access of exopeptidases which are trimming both ends.

Therefore, the MHC class II antigenic peptide of the invention comprising (a) at least the amino acid sequence of the peptide binding motif selected from the group consisting of SEQ ID NOs. 49 to 57 and SEQ ID NOs. 103 to 122, or (b) at least the amino acid sequence of the peptide binding motif selected from the group consisting of SEQ ID NOs. 49 to 57 and SEQ ID NOs. 103 to 122 with additional N- and C-terminal flanking sequences of a corresponding sequence selected from the group consisting of SEQ ID NOs. 1 to 39 and SEQ ID NOs. 58 to 102, preferably comprises additional N- and C-terminal flanking amino acid residues providing additional binding energy.

Preferably, the MHC class II antigenic peptide of the present invention has a binding capacity to the corresponding MHC class II molecule of between one tenth and ten-fold the IC₅₀ of a corresponding peptide selected from the group consisting of SEQ ID NOs. 1 to 39 and SEQ. ID NOs. 58 to 102. The binding capacity of a peptide is measured by determining the concentration necessary to reduce binding of a labelled reporter peptide by 50%. This value is called IC₅₀. A MHC class II antigenic peptide of the invention maintains its binding capacity to the relevant HLA class II molecules as long as it attains IC₅₀ values between one tenth and 10-fold the IC₅₀ of the established reference peptides.

Since peptide trimming occurs in an individual fashion both before and after binding into the peptide binding groove, the occurrence of several truncation variants sharing a common nonameric core region is a common feature of MHC class II-bound peptides.

Importantly, it was shown that C- or N-terminal truncation variants of the same epitope can trigger divergent T cell responses (Arnold et al., (2002) J. Immunol. 169, 739-749).

Several parameters can be envisaged that have an influence on the relative abundance of truncation variants of a particular epitope, e.g. the abundance and integrity of the antigen of relevance, antigen-associated proteins, the abundance of proteases, the type of proteases available and the supply with competitive antigens and/or peptides. Since the antigen supply is a major characteristic that may correlate with the origin of a sample, the ratio of particular truncation variants of an epitope can be of diagnostic value.

A peptide of the invention is a peptide which either has no naturally-occurring counterpart (e.g., such as an mutated peptide antigen), or has been isolated, i.e., separated or purified from components which naturally accompany it, e.g., in tissues such as pancreas, liver, spleen, ovary, testis, muscle, joint tissue, neural tissue, gastrointestinal tissue, or body fluids such as blood, serum, synovial fluid or urine. Typically, the peptide is considered “isolated” when a preparation comprising a peptide of the invention consists to at least 70%, by dry weight of said peptide and to less than 30% of the proteins and naturally-occurring organic molecules with which it is naturally associated. Preferably, a preparation of a peptide of the invention consists of at least 80%, more preferably at least 90%, and most preferably at least 99%, by dry weight, the peptide of the invention. Since a peptide that is chemically synthesized is, by its nature, separated from the components that naturally accompany it, the synthetic peptide is “isolated”.

The invention further provides analogs of the antigenic peptide of the invention. The term analog includes any peptide which displays the functional aspects of these antigenic peptides comprising the binding capacity IC₅₀ and the recognition by antibodies and cells of the immune system. Analogs exhibit essentially the same IC₅₀ as the corresponding reference peptide. The term analog also includes conservative substitutions or chemical derivatives of the peptides.

The term “analog” includes any polypeptide having an amino acid residue sequence substantially identical to the sequences described herein in which one or more residues have been conservatively substituted with a functionally similar residue and which displays the functional aspects of the peptides as described herein. Examples of conservative substitutions include the substitution of one non-polar (hydrophobic) residue such as phenylalanine, tyrosine, isoleucine, valine, leucine or methionine for another, the substitution of one polar (hydrophilic) residue for another such as between arginine and lysine, between glutamine and asparagine, between threonine and serine, the substitution of one basic residue such as lysine, arginine or histidine for another, or the substitution of one acidic residue, such as aspartic acid or glutamic acid for another.

The phrase “conservative substitution” also includes the use of a chemically derivatized amino acid in place of a non-derivatized amino acid. “Chemical derivative” refers to a subject polypeptide having one or more amino acids chemically derivatized by reaction of a functional side group. Examples of such derivatized molecules include for example, those molecules in which free amino groups have been derivatized to form amine hydrochlorides, p-toluene sulfonyl groups, carbobenzoxy groups, t-butyloxycarbonyl groups, chloroacetyl groups, acetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as chemical derivatives are those proteins or peptides, which contain one or more naturally-occurring amino acid derivative of the twenty standard amino acids. For examples: 4-hydroxyproline may be substituted for proline; 5-hydroxylysine may be substituted for lysine; 3-methylhistidine may be substituted for histidine; homoserine may be substituted for serine; and ornithine or citrulline may be substituted for lysine.

The MHC class II antigenic peptides of the invention and the proteins they are derived from can be used as markers in diagnosis of RA and in therapy as anti-RA vaccines. The term marker as used herein refers to a biomolecule, preferably a peptide or a polypeptide, which is expressed in a group of patients with a diagnosed disease, e.g. RA, and attains an abundance that is significantly increased or decreased as compared to a control group.

The marker of the present invention may be used as a prognostic marker to predict the susceptibility to a disease, e.g., to predict the susceptibility to RA, as a diagnostic marker for the diagnosis of a disease, e.g. for the diagnosis of RA, as a differential diagnostic marker to differentiate between different forms of a disease, e.g., to differentiate between different forms of RA, as a prognostic marker for the prediction of the outcome of a disease, e.g., for the prognosis of RA, and as a response marker to determine the efficacy of a therapeutic regime, e.g., as a response marker in the treatment of RA.

In a further embodiment, the MHC class II antigenic peptide comprising (a) at least the amino acid sequence of the peptide binding motif selected from the group consisting of SEQ ID NOs. 49 to 57 and SEQ ID NOs. 103 to 122, or (b) at least the amino acid sequence of the peptide binding motif selected from the group consisting of SEQ ID NOs. 49 to 57 and SEQ ID NOs. 103 to 122, with additional N- and C-terminal flanking sequences of a corresponding sequence selected from the group consisting of SEQ ID NOs. 1 to 39 and SEQ ID NOs. 58 to 102, is used as a marker for erosive and/or non-erosive RA.

In a further embodiment, the MHC class II antigenic peptide comprising (a) at least the amino acid sequence of the peptide binding motif of SEQ ID NO. 49, or (b) at least the amino acid sequence of the peptide binding motif of SEQ ID NO. 49 with additional N- and C-terminal flanking sequences of a corresponding sequence selected from the group consisting of SEQ ID NOs. 1 to 3 is used as a marker for non-erosive RA.

Furthermore, a MHC class II antigenic peptide is provided comprising (a) at least the amino acid sequence of the peptide binding motif of SEQ ID NO. 103, or (b) at least the amino acid sequence of the peptide binding motif of SEQ ID NO. 103 with additional N- and C-terminal flanking sequences of the corresponding sequence of SEQ ID NOs. 58 and 59 is used as a marker for non-erosive RA.

Furthermore, a MHC class II antigenic peptide is provided comprising (a) at least the amino acid sequence of the peptide binding motif of SEQ ID NO. 104, or (b) at least the amino acid sequence of the peptide binding motif of SEQ ID NO. 104 with additional N- and C-terminal flanking sequences of the corresponding sequence of SEQ ID NO. 60 is used as a marker for non-erosive RA.

Furthermore, a MHC class II antigenic peptide is provided comprising (a) at least the amino acid sequence of the peptide binding motif of SEQ ID NO. 105, or (b) at least the amino acid sequence of the peptide binding motif of SEQ ID NO. 105 with additional N- and C-terminal flanking sequences of the corresponding sequence of SEQ ID NO. 61 is used as a marker for non-erosive RA.

Furthermore, a MHC class II antigenic peptide is provided comprising (a) at least the amino acid sequence of the peptide binding motif of SEQ ID NO. 106, or (b) at least the amino acid sequence of the peptide binding motif of SEQ ID NO. 106 with additional N- and C-terminal flanking sequences of the corresponding sequence of SEQ ID NO. 62 is used as a marker for non-erosive RA.

Furthermore, a MHC class II antigenic peptide is provided comprising (a) at least the amino acid sequence of the peptide binding motif of SEQ ID NO. 107, or (b) at least the amino acid sequence of the peptide binding motif of SEQ ID NO. 107 with additional N- and C-terminal flanking sequences of the corresponding sequence of SEQ ID NO. 0.63 is used as a marker for non-erosive RA.

In a further embodiment, the MHC class II antigenic peptide comprising (a) at least the amino acid sequence of the peptide binding motif of SEQ ID NO. 50, or (b) at least the amino acid sequence of the peptide binding motif of SEQ ID NO: 50 with additional N- and C-terminal flanking sequences of the corresponding sequence of SEQ ID NO: 5 is used as a marker for erosive RA.

Furthermore, a MHC class II antigenic peptide is provided comprising (a) at least the amino acid sequence of the peptide binding motif of SEQ ID NO. 108, or (b) at least the amino acid sequence of the peptide binding motif of SEQ ID NO. 108 with additional N- and C-terminal flanking sequences of the corresponding sequence of SEQ ID NOs. 64 to 67 is used as a marker for erosive RA.

Furthermore, a MHC class II antigenic peptide is provided comprising (a) at least the amino acid sequence of the peptide binding motif of SEQ ID NO. 109, or (b) at least the amino acid sequence of the peptide binding motif of SEQ ID NO. 109 with additional N- and C-terminal flanking sequences of the corresponding sequence of SEQ ID NO. 68 is used as a marker for erosive RA.

Furthermore, a MHC class II antigenic peptide is provided comprising (a) at least the amino acid sequence of the peptide binding motif of SEQ ID NO. 110, or (b) at least the amino acid sequence of the peptide binding motif of SEQ ID NO. 110 with additional N- and C-terminal flanking sequences of the corresponding sequence of SEQ ID NOs. 69 and 70 is used as a marker for erosive RA.

Furthermore, a MHC Class II antigenic peptide is provided comprising (a) at least the amino acid sequence of the peptide binding motif of SEQ ID NO. 111, or (b) at least the amino acid sequence of the peptide binding motif of SEQ ID NO. 111 with additional N- and C-terminal flanking sequences of the corresponding sequence of SEQ ID NO. 72 is used as a marker for erosive RA.

Furthermore, a MHC class II antigenic peptide is provided comprising (a) at least the amino acid sequence of the peptide binding motif of SEQ ID NO. 112, or (b) at least the amino acid sequence of the peptide binding motif of SEQ ID NO. 112 with additional N- and C-terminal flanking sequences of the corresponding sequence of SEQ ID NO. 73 is used as a marker for erosive RA.

In a further embodiment, the MHC dass II antigenic peptides of the invention as described above are provided linked to a MHC class II molecule.

Multimers (e.g., dimers, trimers, tetramers, pentamers, hexamers or oligomers) of a class II MHC molecule containing a covalently or non-covalently bound peptide according to the present invention, if conjugated with a detectable label (e.g., a fluorescent moiety, a radionuclide, or an enzyme that catalyzes a reaction resulting in a product that absorbs or emits light of a defined wavelength) can be used to quantify T cells from a subject (e.g., a human patient) bearing cell surface receptors that are specific for, and therefore will bind, such complexes. Relatively high numbers of such T cells are likely to be diagnostic of disease or an indication that the T cells are involved in immunity to the disease. In addition, continuous monitoring of the relative numbers of multimer-binding T cells can be useful in establishing the course of a disease or the efficacy of therapy. Such assays have been developed using tetramers of class I MHC molecules containing an HIV-1-derived or an influenza virus-15 derived peptide (Altman et al. (1996), Science 274:94-96; Ogg et al. (1998), Science 279:2103-21061), and corresponding class II MHC multimers would be expected to be similarly useful. Such complexes could be produced by chemical cross-linking of purified class II MHC molecules assembled in the presence of a peptide of interest or by modification of already established recombinant techniques for the production of class II MHC molecules containing a single defined peptide (Kazono et al. (1994), Nature 369:151-154; Gauthier et al. (1998), Proc. Natl. Acad. Sci. U.S.A. 95:11828-118331). The class II MHC molecule monomers of such multimers can be native molecules composed of full-length alpha and beta chains. Alternatively, they can be molecules containing either the extracellular domains of the alpha and beta chains or the alpha and beta chain domains that form the “walls” and “floor” of the peptide-binding cleft.

The invention also relates to an antibody, fragments or derivatives thereof, directed to and reactive with the above-described MHC class II antigenic peptides. The general methodology for producing antibodies is well known and is disclosed per example in Kohler and Milstein, 1975, Nature 256,494 or in J. G. R. Hurrel, Monoclonal Hybridoma Antibodies: Techniques and Applications, CRC Press Inc., Boco Raron, Fla. (1982). The antibodies can be polyclonal or, preferably, monoclonal, or antibody fragments like be F (ab′) 2, Fab, Fv or scFv. The antibodies of the present invention may also be humanized (Merluzzi S. et al., (2000), Adv. Clin. Path., 4(2): 77-85) or human antibodies (Aujame L. et al., Hum. Antibodies, (1997), 8(4): 155-168).

The present invention also provides a nucleic acid molecule encoding a MHC class II antigenic peptide of the invention comprising (a) at least the amino acid sequence of the peptide binding motif selected from the group consisting of SEQ ID NOs. 49 to 57 and SEQ ID NOs. 103 to 122, or (b) at least the amino acid sequence of the peptide binding motif selected from the group consisting of SEQ ID NOs. 49 to 57 and SEQ ID NOs. 103 to 122, with additional N- and C-terminal flanking sequences of a corresponding sequence selected from the group consisting of SEQ ID NOs. 1 to 39 and SEQ. ID NOs. 58 to 102. Preferably, the nucleic acid molecule is a DNA molecule.

Furthermore, a nucleic acid molecule is provided encoding a MHC class II antigenic peptide of the invention linked to a MHC class II molecule.

This invention also provides a recombinant nucleic acid construct comprising the nucleic acid molecules as described above, operably linked to an expression vector. Expression vectors suitable for use in the present invention comprise at least one expression control element operably linked to the nucleic acid sequence encoding the antigenic peptide or the antigenic peptide linked to a MHC class II molecule. The recombinant expression construct may be a DNA construct.

The expression control elements are inserted in the vector to control and regulate the expression of the nucleic add sequence encoding the antigenic peptide of the invention. Examples of expression control elements include, but are not limited to, lac system, operator and promoter regions of phage lambda, yeast promoters and promoters derived from polyoma, adenovirus, retrovirus or SV40. Additional preferred or required operational elements include, but are not limited to, leader sequence, termination codons, polyadenylation signals and any other sequences necessary or preferred for the appropriate transcription and subsequent translation of the nucleic acid sequence in the host system. It will be understood by one skilled in the art that the correct combination of required or preferred expression control elements will depend on the host system chosen. It will further be understood that the expression vector should contain additional elements necessary for the transfer and subsequent replication of the expression vector containing the nucleic acid sequence in the host system. Examples of such elements include, but are not limited to, origins of replication and selectable markers. It will further be understood by one skilled in the art that such vectors are easily constructed using conventional methods (“DNA Isolation and Sequencing”, Bruce A. Roe, Judy S. Crabtree and Akbar S. Khan, Published by John Wiley & Sons, 1996) or are commercially available.

Another aspect of this invention relates to a host organism or a host cell into which a recombinant nucleic acid construct comprising the nucleic acid molecules as described above, operably linked to an expression vector, has been inserted. The host cells transformed with the nucleic acid constructs encompassed by this invention include eukaryotes, such as animal, plant, insect and yeast cells and prokaryotes, such as E. coli. The means by which the nucleic add construct carrying the nucleic acid sequence may be introduced into the cell include, but are not limited to, microinjection, electroporation, transduction, or transfection using DEAE-dextran, lipofection, calcium phosphate or other procedures known to one skilled in the art (Sambrook et al. (1989) in “Molecular Cloning. A Laboratory Manual”, Cold Spring Harbor Press, Plainview, N.Y.).

In a preferred embodiment, eukaryotic expression vectors that function in eukaryotic cells are used. Examples of such vectors include, but are not limited to, retroviral vectors, vaccinia virus vectors, adenovirus vectors, herpes virus vector, fowl pox virus vector, plasmids, or the baculovirus transfer vectors. Preferred eukaryotic cell lines include, but are not limited to, COS cells, CHO cells, HeLa cells, NIH/3T3 cells, 293 cells (ATCC# CRL15731), T2 cells, dendritic cells, monocytes or Epstein-15 Barr Virus transformed B cells.

An antigenic peptide of the invention can be obtained, for example, by extraction from a natural source (e.g., elution from MHC II molecules); by expression of a recombinant nucleic acid encoding the peptide; or by chemical synthesis. A peptide that is produced in a cellular system different from the source from which it naturally originates is “isolated,” because it will be separated from components which naturally accompany it. The recombinant peptide expressed by a host organism can be obtained as a crude lysate or can be purified by standard protein purification procedures known in the art which may include differential precipitation, size exclusion chromatography, ion-exchange chromatography, isoelectric focusing, gel electrophoresis, affinity, and immunoaffinity chromatography and the like. The extent of isolation or purity can be measured by any appropriate method, e.g. mass spectrometry or HPLC analysis. The peptides may be prepared synthetically by procedures described in Merrifield, (1986) Science 232: 341-347, and Barany and Merrifield, The Peptides, Gross and Meienhofer, eds (N.Y., Academic Press), pp. 1-284 (1979). The synthesis can be carried out in solution or in solid phase or with an automatized synthesizer (Stewart and Young, Solid Phase Peptide Synthesis, 2nd ed., Rockford Ill., Pierce Chemical Co. (1984)).

Therefore, the present invention further provides a method for producing a MHC class II antigenic peptide comprising (a) at least the amino acid sequence of the peptide binding motif selected from the group consisting of SEQ ID NOs. 49 to 57 and SEQ ID NOs. 103 to 122, or (b) at least the amino acid sequence of the peptide binding motif selected from the group consisting of SEQ ID NOs. 49 to 57 and SEQ ID NOs. 103 to 122, with additional N- and C-terminal flanking sequences of a corresponding sequence selected from the group consisting of SEQ ID NOs. 1 to 39 and SEQ ID NOs. 58 to 102, comprising the steps of culturing the host cell containing a recombinant nucleic acid construct as described above under conditions allowing expression of said peptide and recovering the peptide from the cells or the culture medium.

In a further embodiment of the present invention, a method is provided for isolating and identifying MHC class II associated RA antigenic peptides in femtomolar amounts, which method comprises (a) providing immature dendritic cells in a number comprising 0.1 to 5 μg MHC class II molecules; (b) contacting the cells of (a) with serum or synovial fluid and inducing maturation of dendritic cells by adding TNFalpha; (c) isolating class II MHC molecule-antigenic peptide complexes from the cells with methods comprising solubilization of the cells and sequestration of the complexes of MHC class II molecules with antigenic peptides by immunoprecipitation or immunoaffinity chromatography, (d) washing the sequestered complexes of MHC class II molecules with antigenic peptides with water in an ultrafiltration tube; (e) eluting the associated antigenic peptides from the MHC class II molecules at 37° C. with diluted trifluoro acetic acid, and (f) separating, detecting and identifying the isolated peptides by liquid chromatography and mass spectrometry. Furthermore, in step (f) of the method, the liquid chromatography comprises a first linear elution step from the reversed-phase material with a volume sufficient to elute the majority of contaminants prior to peptide elution. Moreover, the method may further comprise (g) analyzing the identified peptides by methods comprising a database and a software developed to perform comparative data analysis across multiple datasets.

The amount of tissue or bodily fluid necessary to obtain e.g. 100 ng MHC class II molecules depends on the number of cells that do express MHC class II and on the expression rate of MHC class II molecules: e.g. 100 ng of MHC class II are equivalent to about 2×10⁵ mature DCs or 5 to 10×10⁶ peripheral blood monocytes or about 5×10⁷ peripheral blood mononuclear cells which can be obtained from about 50 ml of blood.

For the purification of class II MHC molecule-antigenic peptide complexes from cells or tissue, the membranes of the cells or tissue have to be solubilized. Cell lysis may be carried out with methods known in the art, e.g. freeze-and-thaw cycles and the use of detergents, and combinations thereof. Preferred lysis methods are solubilization using detergents, preferably TX-100, NP40, n-octylglucoside, Zwittergent, Lubrol, CHAPS, most preferably TX-100 or Zwittergent 3-12. Cell debris and nuclei have to be removed from cell lysates containing the solubilized receptor-peptide complexes by centrifugation. Therefore, the complexes of class II MHC molecules with antigenic peptides are isolated from the cells with methods comprising solubilization with a detergent.

Furthermore, the MHC class II molecule-peptide complexes are purified from cell lysates by methods comprising immunoprecipitation or immunoaffinity chromatography. For the immunoprecipitation or immunoaffinity chromatography, antibodies specific for MHC class II molecules and suitable for these methods are used. The specific antibodies are preferably monoclonal antibodies, and are covalently or non-covalently e.g. via Protein A, coupled to beads, e.g. sepharose or agarose beads. A selection of the broad panel of anti-HLA antibodies used in the prior art comprises: anti-HLA-DR antibodies: L243, TU36, DA6.147, preferably L243; anti-HLA-DQ antibodies: SPVL3, TU22, TU169, preferably TU22 and TU169; anti-HLA-DP antibody B7/21 and anti-HLA-A,B,C antibodies W6/32 and B9.12.

Monoclonal antibodies specific for different MHC class II molecules may be commercially obtained (e.g. Pharmingen, Dianova) or purified from the supernatant of the respective hybridoma cells using Protein A- or Protein G-affinity chromatography. Purified monoclonal antibodies may be coupled by various methods known in the art, preferably by covalently coupling antibody amino groups to CNBr-activated sepharose.

Immunoisolation of MHC molecules may be performed by incubating the antibody-beads with the cell lysate under rotation for several hours or chromatographically by pumping the cell lysate through a micro-column. Washing of the antibody-beads may be performed in eppendorf tubes or in the microcolumn. The efficacy of the immunoprecipitation may be analysed by SDS-PAGE and western blotting using antibodies recognizing denatured MHC molecules (anti-HLA-DRalpha: 1B5; anti-HLA class I: HC10 or HCA2).

The sequestered MHC class II molecule-peptide complexes are washed with water or low-salt buffer before elution in order to remove residual detergent contaminants. The low salt buffer may be a Tris, phosphate or acetate buffer in a concentration range of 0.5-10 mM, preferably in a concentration of 0.5 mM. In a more preferred embodiment, the MHC class II molecule-peptide complexes are washed with ultrapure water (sequencing grade) conventionally used for HPLC analysis, preferably with ultrapure (sequencing grade) water from MERCK. The washing step may be carried out by ultrafiltration. The ultrafiltration may be carried out in an ultrafiltration tube with a cut-off of 30 kD, 20 kD, 10 kD or 5 kD, preferably of 30 kD and a tube volume of 0.5-1.0 ml (“Ultrafree” tubes; Millipore). The washing in the ultrafiltration tube may be carried out 4 to 12 times, preferably 6 to 10 times, with a volume of 16 to 20 times the volume of the beads carrying the receptor-peptide complexes, preferably with a volume of 15 times the beads. The eluted peptides may be separated from the remaining MHC class II molecules using the same-ultrafiltration tube. The eluted peptides may then be lyophilized.

By eluting the peptides from the MHC class II molecules, a complex mixture of naturally processed peptides derived from the source of potential antigen and from polypeptides of intra- or extracellular origin, is obtained. Only after elution, peptides can be separated and subjected to sequence analysis.

The antigenic peptides in the method of the present invention may be eluted by a variety of methods known in the art, preferably by using diluted add, e.g., diluted acetonitrile (Jardetzky T S et al., Nature 1991 353, 326-329), diluted acetic acid and heating (Rudensky A Y et al., Nature 1991, 353, 622-626; Chicz R M et al., Nature 1992, 358, 764-768) or diluted trifluoro acetic acid at 37° C. (Kropshofer H et al., J Exp Med 1992, 175, 1799-1803). Most preferably, the peptides are eluted at 37° C. with diluted trifluoro acetic acid.

The isolated antigenic peptides are then separated, detected and identified. By detecting it is understood that the amino acid sequence of the individual peptides in the mixture of isolated antigenic peptides is elucidated by methods adequate to detect and sequence femtomolar amounts of peptides. By identifying it is understood that it is established from which proteins or polypeptides the antigenic peptides are derived and which sequence they constitute within these proteins or polypeptides.

In a first step, the complex mixture of eluted peptides may be separated by one of a variety of possible chromatographic methods, e.g. by reversed phase, anion exchange, cation exchange chromatography or a combination thereof. Preferably, the separation is performed by C18-reverse phase chromatography or by reversed-phase/cation exchange two-dimensional HPLC, denoted as MudPit (Washburn M P et al., Nat. Biotechnol., (2001), 19, 242-247).

The separation is done in a HPLC mode utilizing fused-silica micro-capillary columns which are either connected to a nano-flow electrospray source of a mass spectrometer or to a micro-fractionation device which spots the fractions onto a plate for MALDI analysis.

Liquid chromatography comprises peptide fractionation by the use of a strong ion exchange material and a hydrophobic reversed-phase material. For the elution of the peptides from the ion exchange and reversed-phase material different elution programs are run one after another comprising elutions with salt and with organic solvents, e.g., acetonitrile. The elution from the reversed-phase material is conducted in several steps of linear gradients of different lengths and slopes. A contamination in the sample to be fractionated may be any contamination whose elution competes with the detection of the peptide peaks in the mass spectrometer. Therefore, in order to prevent simultaneous elution, the contaminants have to be eluted with a sufficient solvent volume prior to the peptide elution step. Depending on the column used for liquid chromatography the solvent volume sufficient to elute the contaminants prior to the peptide elution step may be 100 to 200 times the column volume.

A variety of mass spectrometric techniques are suitable, preferably MALDI-post source decay (PSD) MS or electrospray ionization tandem mass spectrometry (ESI-MS), most preferably ion-trap ESI-MS.

The sequences of the individual peptides can be determined by means known in the art. Preferably, sequence analysis is performed by fragmentation of the peptides and computer-assisted interpretation of the fragment spectra using algorithms, e.g. MASCOT or SEQUEST. Both computer algorithms use protein and nucleotide sequence databases to perform cross-correlation analyses of experimental and theoretically generated tandem mass spectra. This allows automated high through-put sequence analysis.

The isolated and identified antigenic peptides of the invention can be validated by the MHC binding motif, the MHC binding capacity and/or by T cell recognition.

MHC Binding Motif

Peptides associated to a particular MHC molecule (allelic variant) have common structural characteristics, denoted as binding motifs, necessary to form stable complexes with MHC molecules. Peptide ligands eluted from MHC class I molecules are relatively short, ranging from 8-11 amino acids. Moreover, 2 or 3 side chains of the peptide are relevant for binding. The position of the respective amino acid side chains varies with the HLA allele, most often two of these so-called “anchor” residues are located at positions 2 and 9. With respect to a particular anchor position, only 1 or 2 amino acids normally can function as anchor amino acids e.g. leucine or valine V at position 2 in the case of HIA-A2.

In the case of MHC class II molecules, the peptide length varies from 11 to 25 amino acids, as longer peptides can bind since both ends of the peptide binding groove are open. Most HLA class II molecules accommodate up to 4 anchor residues at relative positions P1, P4, P6 and P9 contained in a nonameric core region. This core region, however, can have variable distance from the N-terminus of the peptide. In the majority of cases, 2-4 N-terminal residues precede the core region. Hence, the P1 anchor residues is located at positions 3, 4 or 5 in most HLA class II associated peptides. Peptides eluted from HLA-DR class II molecules share a big hydrophobic P1 anchor, represented by tyrosine, phenylalanine, tryptophane, methionine, leucine, isoleucine or valine.

The position and the exact type of anchor residues constitute the peptide binding motif which is known for most of the frequently occurring HLA class II allelic products. A computer algorithm allowing motif validation in peptide sequences is “Tepitope”, available by vaccinome.

MHC Binding Capacity

Peptides identified by the method of the invention may be tested for their ability to bind to the appropriate MHC class II molecule by methods known in the art using, for example, isolated MHC class II molecules and synthetic peptides with amino acid sequences identical to those identified by the method of the invention (Kropshofer H et al., J. Exp. Med. 1992; 175, 1799-1803; Vogt A B et al., J. Immunol. 1994; 153, 1665-1673; Sloan V S et al., Nature 1995; 375, 802-806). Alternatively, a cellular binding assay using MHC class II expressing cell lines and biotinylated peptides can be used to verify the identified epitope (Arndt S O et al., EMBO J., 2000; 19, 1241-1251)

In both assays, the relative binding capacity of a peptide is measured by determining the concentration necessary to reduce binding of a labelled reporter peptide by 50%. This value is called IC₅₀. Peptide binding with a reasonable affinity to the relevant HLA class II molecules attain IC₅₀ values not exceeding 10-fold the IC₅₀ of established reference peptides.

The same binding assays can also be used to test the ability of peptides to bind to alternative class II MHC molecules, i.e., class II MHC molecules other than those from which they were eluted using the method of the invention. The diagnostic methods of the invention using such peptides and therapeutic methods of the invention, using either the peptides or peptides derived from them, can be applied to subjects expressing such alternative class II MHC molecules.

T Cell Recognition

The epitope verification procedure may involve testing of peptides identified by the method of the invention for their ability to activate CD4+ T cell populations. Peptides with amino add sequences either identical to those identified in the present invention or corresponding to a core sequence derived from a nested group of peptides identified in the present invention are synthesized. The synthetic peptides are then tested for their ability to activate CD4+ T cells from (a) test subjects expressing the MHC class II molecule of interest and having at least one symptom of the disease; and (b) control subjects expressing the MHC class II molecule of interest and having no symptoms of the disease. Additional control subjects can be those with symptoms of the disease and not expressing the MHC class II molecule of interest.

In some diseases (e.g., those with an autoimmune component) responsiveness in the CD4+ T cells of test subjects but not in CD4+ T cells of the control subjects described in (b) provides confirmatory evidence that the relevant peptide is an epitope that activates CD4+ T cells that can initiate, promote, or exacerbate the relevant disease. In other diseases (e.g., cancer or infectious diseases without an autoimmune component), a similar pattern of responsiveness and non-responsiveness to that described in the previous sentence would indicate that the relevant peptide is an epitope that activates CD4+ T cells that can mediate immunity to the disease or, at least, a decrease in the symptoms of the disease.

CD4+ T cell responses can be measured by a variety of in vitro methods known in the art. For example, whole peripheral blood mononuclear cells (PBMC) can be cultured with and without a candidate synthetic peptide and their proliferative responses measured by, e.g., incorporation of [³H]-thymidine into their DNA. That the proliferating T cells are CD4+ T cells can be tested by either eliminating CD4+ T cells from the PBMC prior to assay or by adding inhibitory antibodies that bind to the CD4+ molecule on the T cells, thereby inhibiting proliferation of the latter. In both cases, the proliferative response will be inhibited only if CD4+ T cells are the proliferating cells. Alternatively, CD4+ T cells can be purified from PBMC and tested for proliferative responses to the peptides in the presence of APC expressing the appropriate MHC class II molecule. Such APC can be B-lymphocytes, monocytes, macrophages, or dendritic cells, or whole PBMC. APC can also be immortalized cell lines derived from B-lymphocytes, monocytes, macrophages, or dendritic cells. The APC can endogenously express the MHC class II molecule of interest or they can express transfected polynucleotides encoding such molecules. In all cases the APC can, prior to the assay, be rendered non-proliferative by treatment with, e.g., ionizing radiation or mitomycin-C.

As an alternative to measuring cell proliferation, cytokine production by the CD4+ T cells can be measured by procedures known to those in art. Cytokines include, without limitation, interleukin-2 (IL-2), interferon-gamma (IFN-gamma), interleukin-4 (IL-4), TNF-alpha, interleukin-6 (IL-6), interleukin-10 (IL-10), interleukin-12 (IL-12) or TGF-beta. Assays to measure them include, without limitation, ELISA, and bio-assays in which cells responsive to the relevant cytoline are tested for responsiveness (e.g., proliferation) in the presence of a test sample.

Alternatively, cytokine production by CD4+ lymphocytes can be directly visualized by intracellular immunofluorescence staining and flow cytometry.

Moreover, the MHC class II antigenic peptides of the present invention may be used in the diagnosis of RA. Therefore, a further embodiment of the invention is the use of an antigenic peptide according to the present invention as a marker for RA.

Preferably, a MHC class II antigenic peptide comprising (a) at least the amino acid sequence of the peptide binding motif selected from the group consisting of SEQ ID NOs. 49 to 57 and SEQ ID NOs. 103 to 122, or (b) at least the amino acid sequence of the peptide binding motif selected from the group consisting of SEQ ID NOs. 49 to 57 and SEQ ID NOs. 103 to 122, with additional N- and C-terminal flanking sequences of a corresponding sequence selected from the group consisting of SEQ ID NOs. 1 to 39 and SEQ ID NOs. 58 to 102, is used as a marker for RA.

In another embodiment, the antigenic peptides of the invention may be used as response markers to track the efficacy of a therapeutic regime. Essentially, a baseline value for an antigenic peptide can be determined, then a given therapeutic agent is administered, and the levels of the antigenic peptide are monitored subsequently, whereas a change in the level of the antigenic peptide is indicative of the efficacy of a therapeutic treatment.

Furthermore, the antigenic peptides which are only found in certain stages or phases of a disease, preferably of RA, may be utilized as stage-specific markers. Essentially, the levels of the antigenic peptides which have been linked to a certain disease stage are monitored regularly, thereby providing information about the stage of the disease and its progression.

The invention also includes the use of the polypeptides the RA antigenic peptides are derived from as markers for the diagnosis and monitoring of a disease, preferably of RA, and in particular, of erosive versus non-erosive RA. The rationale for the use of the respective proteins is that DCs reside in most tissues where they capture exogenous antigens via specific receptors and via specialized endocytotic mechanisms (e.g. macropinocytosis) followed by presentation of the processed antigens as peptides on MHC class II molecules. Previous studies have shown that the frequency of a peptide epitope found in the context of MHC class II molecules, e.g. the RA antigenic peptides, in the majority of cases mirrors the abundance of the protein from which this particular peptide was derived from. Therefore, not only the RA antigenc peptides but also the corresponding proteins can serve as markers for RA.

Therefore, in a further embodiment of the present invention, the use of a polypeptide selected from the group consisting of interferon-gamma-inducible lysosomal thiol reductase (SEQ ID NO: 40), integrin beta-2 (SEQ ID NO. 123), phosphatitylinositol-4,5-bisphosphate 3-kinase (SEQ ID NO: 124), urokinase-type plasminogen activator (SEQ ID NO. 125), immunoglobulin heavy chain V-III region (V_{H}26) (SEQ ID NO. 126), DJ-1 protein (SEQ ID NO. 127), apolipoprotein B-100 (SEQ ID NO: 41), 26S proteasome non-ATPase regulatory subunit 8 (SEQ ID NO. 128), interleukin-1 receptor (SEQ ID NO: 129), fibromodulin (SEQ ID NO. 130), GM-CSF/IL-3/IL-5 receptor (SEQ ID NO. 131), sorting nexin 3 (SEQ ID NO. 132), inter-alpha-trypsin inhibitor heavy chain H4 (SEQ ID NO: 42), complement C4 (SEQ ID NO: 43), complement C3 (SEQ ID NO: 44), SH3 domain-binding glutamic acid-rich-like protein 3 (SEQ ID NO: 45), interleukin-4-induced protein 1 (SEQ ID NO: 46), hemopexin (SEQ ID NO: 47), Hsc70-interacting protein (SEQ ID NO: 48), invariant chain (Ii) (SEQ ID NO. 133), retinoic acid receptor responder protein 2 (SEQ ID NO. 134), fibronectin (SEQ ID NO. 135), cathepsin B (SEQ ID NO: 136), tripeptidyl-peptidase II (SEQ ID NO. 137), legumain (SEQ ID NO. 138), platelet activating factor receptor (SEQ ID NO. 139), poly-alpha-2.8-sialyltransferase (SEQ ID NO. 140), ras-leated protein Rab-11B (SEQ ID NO. 141) as a marker for RA is provided. Preferably, the polypeptide is used as a marker for erosive RA. It is also preferred to use the polypetide as a marker for non-erosive RA. Especially preferred is the use of interleukin-4-induced protein 1 (SEQ ID NO: 46) as a marker for RA. The FIG. 1 polypeptide has not been known as a marker for RA until now, and is considered as an important candidate marker for RA.

The diagnosis of RA can be made by examining expression and/or composition of a polypeptide or peptide marker for RA, by a variety of methods, including enzyme linked immunosorbent assays (ELISAs), Western blots, immunoprecipitations and immunofluorescence. A test sample from an individual is assessed for the presence of an alteration in the expression and/or an alteration in composition of a polypeptide or a peptide of the present invention. An alteration in expression of a polypeptide or peptide can be, for example, an alteration in the quantitative polypeptide expression (i.e., the amount of polypeptide produced); an alteration in the composition of a polypeptide is an alteration in the qualitative polypeptide expression (e.g., expression of a mutant polypeptide or of a different splicing variant).

Both such alterations (quantitative and qualitative) can also be present. An “alteration” in the polypeptide expression or composition, as used herein, refers to an alteration in expression or composition in a test sample, as compared with the expression or composition of the peptide or polypeptide in a control sample. A control sample is a sample that corresponds to the test sample (e.g., is from the same type of cells), and is from an individual who is not affected by RA. An alteration in the expression or composition of the peptide or polypeptide in the test sample, as compared with the control sample, is indicative of RA or a susceptibility to RA. Various means of examining expression or composition of a peptide or polypeptide of the present invention can be used, including spectroscopy, colorimetry, electrophoresis, isoelectric focusing, and immunoassays (e.g., David et al., U.S. Pat. No. 4,376,110) such as immunoblotting (see also Current Protocols in Molecular Biology, particularly chapter 10). For example, in one embodiment, an antibody capable of binding to the polypeptide (e.g., as described above), preferably an antibody with a detectable label, can be used. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

Western blotting analysis, using an antibody as described above that specifically binds to a peptide or polypeptide of the present invention, may be used to measure the level or amount of a peptide or polypeptide in a test sample and comparing it with the level or amount of the peptide or polypeptide in a control sample. Preferably the peptide or polypeptide in a test sample is measured in a homogenous or a heterogenous immuno assay. A level or amount of the polypeptide in the test sample that is higher or lower than the level or amount of the polypeptide in the control sample, such that the difference is statistically significant, is indicative of an alteration in the expression of the polypeptide, and is diagnostic for a RA or a susceptibility to RA.

Therefore, the present invention also relates to a diagnostic composition comprising an antibody reactive with a MHC class II antigenic peptide of the invention.

In a further embodiment the antigenic peptides of the invention or the proteins they are derived from may be used in the prevention and treatment of a disease, preferably of RA.

One aspect of the invention is a therapeutic purpose, wherein one or more of the identified antigenic peptides are used to vaccinate patients against RA, preferably against erosive and/or non-erosive RA. In the course of the vaccination the antigenic peptide would induce an antigen-specific T cell tolerance in the patient which would ultimately lead to regression of the disease or to an attenuation of disease development.

A promising strategy to induce specific immune tolerance in future clinical trials is the use of DNA tolerizing vaccines. DNA tolerizing vaccines encoding autoantigens alone were shown to reduce T cell proliferative responses (Ruiz, P. et al., J Immunol 162 (1999) 3336-3341), while DNA tolerizing vaccines co-delivering autoantigen plus IL-4 also induced protective T_{H}2 responses (Garren, H. et al., Immunity 15 (2001) 15-22). Examples of non-polynucleotide-specific tolerizing therapies under development include protein antigens, naturally processed peptides, altered peptide ligands, other biomolecules, such as DNA, or proteins and peptides containing posttranslational modifications, and antigens delivered orally to induce “oral tolerance” (reviewed in: Robinson, W. H. et al., Clin Immunol 103 (2002) 7-12). A potential adverse effect with regard to tolerizing therapies is the development of autoimmunity.

To this end, the relevant RA antigenic peptides may be directly administered to the patient in an amount sufficient for the peptides to bind to the MHC molecules, and provoke peripheral tolerance of T cells.

Alternatively, the antigenic peptides of the invention may be utilized for the generation of vaccines based on DCs. In this case, autologous DCs derived from patients' monocytes may be pulsed with the relevant peptides or recombinant proteins containing the relevant peptide sequences.

Therefore, the present invention provides a pharmaceutical composition comprising a MHC class II antigenic peptide comprising (a) at least the amino acid sequence of the peptide binding motif selected from the group consisting of SEQ ID NOs. 49 to 57 and SEQ ID NOs. 103 to 122, or (b) at least the amino acid sequence of the peptide binding motif selected from the group consisting of SEQ ID NOs. 49 to 57 and SEQ ID NOs. 103 to 122, with additional N- and C-terminal flanking sequences of a corresponding sequence selected from the group consisting of SEQ ID NOs. 1 to 39 and SEQ ID NOs. 58 to 102, an antibody reactive with said antigenic peptide, or a polypeptide selected from the group consisting of SEQ ID NOs 40 to 48 and SEQ. ID NOs. 123 to 141, and optionally a pharmaceutically acceptable excipient, diluent or carrier. The antigenic peptide has to be present in an amount sufficient to tolerize specific lymphocytes. Such an amount will depend on the peptide used, the administration, the severity of the disease to be treated and the general conditions of the patient and will usually range from 1 to 50 mg/ml, for example in case of peptides being loaded on dendritic cells.

An acceptable excipient, diluent or carrier may be phosphate buffered saline for in vitro studies and physiological salt solutions for in vivo applications.

“Vaccination” herein means both active immunization, i.e. the in vivo administration of the peptides to elicit an in vivo immune tolerance directly in the patient and passive immunization, i.e. the use of the peptides to tolerize in vitro CD4+ T lymphocytes or to stimulate autologous or allogeneic dendritic cells, which are subsequently re-inoculated into the patient.

The present invention also provides the antigenic peptides, antibodies, nucleic acids, host cells, methods, compositions and uses substantially as herein before described especially with reference to the Examples.

Having now generally described this invention, the same will become better understood by reference to the specific examples, which are included herein for purpose of illustration only and are not intended to be limiting unless otherwise specified, in connection with the following figures.

### EXAMPLES

The examples below are illustrated in connection with the figures described above and based on the methodology summarized in FIG. 1, as described in the following. Commercially available reagents referred to in the examples, were used according to manufacturer's instructions unless otherwise indicated.

Methodology of the Invention

Dendritic Cells and Culturing The study was performed with human dendritic cells which were differentiated from monocytes, as described below. Monocytes were purified from human peripheral blood. The blood was taken from healthy donors with the following haplotypes: (1) HLA-DRB1*0401, *03011, (2) HLA-DRB1*0401, *0304, (3) HLA-DRB1*0401, *1301, (4) HLA-DRB1*0401, *0701, HLA-DRB1*0401, *0407.

All cells were cultured in RPMI 1640 medium (short: RPMI) supplemented with 1 mM Pyruvate, 2 mM Glutamine and 10% heat-inactivated fetal calf serum (Gibco BRL, Rockville, Md.).

Isolation of Peripheral Blood Mononuclear Cells (PBMCs)

Peripheral blood was obtained from the blood bank in Mannheim, Germany as standard buffy coat preparations from healthy donors. Heparin (200 I.U./ml blood, Liquemine, Roche) was used to prevent clotting. Peripheral blood mononuclear cells (PBMCs) were isolated by centrifugation in LSM® (1.077-1.080 g/ml; ICN, Aurora, Ohio) at 800 g (room temperature) for 30 min. PBMCs were collected from the interphase and washed twice in RPMI containing 20 mM Hepes (500 g for 15 min, 300 g for 5 min). In order to remove erythrocytes, PBMCs were treated with ALT buffer (140 mM ammonium chloride, 20 mM Tris, pH 7.2) for 3 min at 37° C. PBMCs were washed twice with RPMI containing 20 mM Hepes (200 g for 5 min).

Generation of Dendritic Cells from Peripheral Blood Monocytes.

Monocytes were isolated from PBMCs by positive sorting using anti-CD14 magnetic beads (Miltenyi Biotech, Auburn, Calif.) according to the manufacturer's protocol. Monocytes were cultured in RPMI supplemented with 1% non-essential amino acids (Gibco, BRL, Rockville, Md.), 50 ng/ml recombinant human granulocyte macrophage-colony stimulating factor (GM-CSP; S.A. 1.1×10⁷ U/mg) (Leucomax; Novartis, Basel Switzerland) and 3 ng/ml recombinant human IL-4 (S.A. 2.9×10⁴ U/μg) (R&D Systems, Minneapolis, Minn.). Monocytes were seeded at 0.3×10⁶/ml in 6-well plates (Costar) for 5 days to obtain immature dendritic cells.

The quality of monocyte-derived immature dendritic cells was routinely monitored by flow-cytometric analysis and assessed to be appropriate when they displayed the following phenotype: CD1a (high), CD3 (neg.), CD14 (low), CD19 (neg.), CD56 (neg.), CD80 (low), CD83 (neg.), CD86 (low) and HLA-DR (high). In contrast, mature dendritic cells (cf. below) display the following phenotype: CD1a (low), CD80 (high), CD83 (high), CD86 (high) and HLA-DR (high). Monoclonal antibodies against CD1a, CD3, CD14, CD19, CD56, CD80, CD83, CD86 as well as the respective isotype controls were purchased from Pharmingen (San Diego, Calif.).

Exposure of Dendritic Cells to Serum or Synovial Fluid

Serum and synovial fluid were irradiated for 30 min with ¹³⁷Cs (70 TBq). To feed dendritic cells with serum- or synovia-derived antigen, 6×10⁶ immature dendritic cells were pulsed with either 1 ml serum or 0.6 ml synovial fluid. At the same time maturation of dendritic cells was induced by adding 10 ng/ml recombinant human tumor necrosis factor alpha (TNFα; S.A. 1.1×10⁵ U/μg). As a control, 6×10⁶ immature dendritic cells were incubated with TNFα alone.

After 24 hrs in culture, mature dendritic cells were harvested by centrifugation at 300 g for 10 min. Cells were washed with PBS and transferred to an eppendorf tube. After centrifugation at 400 g for 3 min, the supernatant was completely removed and the cells were frozen at −70° C.

Generation of Anti-HLA Class II Beads

The anti-HLA-DR monoclonal antibody (mAb) L243 (ATCC, Manassas, Va.) was produced by culturing the respective mouse hybridoma cell line. mAb L243 was purified using ProteinA sepharose (Pharmacia, Uppsala, Sweden) and immobilized to CNBr-activated sepharose beads (Pharmacia) at a final concentration of 2.5 mg/ml, according to the manufacturer's protocol. L243 beads were stored in PBS containing 0.1% Zwittergent 3-12 (Calbiochem, La Jolla, Calif.).

Nano-Scale Purification of HLA-DR-Peptide Complexes

Pellets of frozen dendritic cells were resuspended in 10-fold volume of ice cold lysis buffer (1% Triton-X-100, 20 mM Tris, pH 7.8, 5 mM MgCl₂, containing protease inhibitors chymostatin, pepstatin, PMSF and leupeptin (Roche, Mannheim, Germany)) and lysed in a horizontal shaker at 1000 rpm, 4° C. for 1 h. The cell lysate was cleared from cell debris and nuclei by centrifugation at 10000 g, 4° C. for 10 min. The lysate was co-incubated with L243 beads (5-10 μL243 beads per 100 μl cell lysate) in a horizontal shaker at 1000 rpm, 4° C. for 2 hrs. Immunoprecipitated HLA-DR-peptide complexes bound to L243 beads were sedimented by centrifugation at 1000 g, 4° C. for 1 min and washed four times with 500 μl 0.1% Zwittergent 3-12 (Calbiochem) in PBS.

The efficacy of depletion of HLA-DR-peptide complexes was monitored by analyzing the respective cell lysates before and after immunoprecipitation and aliquots of the beads by western blotting using the anti-HLA-DRα-specific mAb 1B5 (Adams, T. E. et al., Immunology 50 (1983) 613-624).

Elution of HLA-DR-Associated Peptides

HLA-DR-peptide complexes bound to L243 beads were resuspended in 100 μl H₂O (HPLC-grade; Merck, Darmstadt, Germany), transferred to an ultrafiltration tube, Ultrafree MC, 30 kD cut-off (Millipore, Bedford, Mass.) and washed 10 times with 100 μl H₂O(HPLC-grade) by centrifugation for 1-2 min at 10000 g at RT. For eluting the bound peptides, 60 μl 0.1% trifluoracetic acid (Fluka, Buchs, Switzerland) in H₂O(HPLC-grade) was added and incubation was performed for 30 min at 37° C. Eluted peptides were collected in a new eppendorf tube by centrigugation of the Ultrafree unit at 10000 g for 3 min at RT and immediately lyophilized in a Speed-Vac™ vacuum centrifuge.

Fractionation by Two-Dimensional Nanoflow LC

To perform high-throughput sequencing of complex peptide mixtures, the MudPIT (multidimensional protein identification technology) was used (Washburn, M. P. et al., Nat Biotechnol 19 (2001), 242-247) which is based on liquid chromatographic fractionation followed by mass spectrometric sequence determination.

To this end, lyophilized peptides eluted from HLA molecules were resuspended in a buffer containing 5% (v/v) acetonitrile (ACN), 0.5% (v/v) acetic acid, 0.012% (v/v) heptafluoro butyric acid (HFBA) and 1% (v/v) formic acid. The peptide mixture was fractionated on a fused-silica microcapillary column (100 μm i.d.×375 μm) generated by a Model P-2000 laser puller (Sutter Instrument Co., Novato, Calif.). The microcolumn was packed with 3 μm/C18 reversed-phase material (C18-ACE 3 μm [ProntoSIL 120-3-C18 ACE-EPS, Leonberg, Germany]) followed by 3 cm of 5 μm cation exchange material (Partisphere SCX; Whatman, Clifton, USA).

A fully automated 8-step gradient separation on a LC Packings UltiMate HPLC (LC Packings, San Francisco, USA) was carried out, using the following buffers: 5% ACN/0.012% HFBA/0.5% acetic acid (buffer A), 80% ACN/0.012% HFBA/0.5% acetic acid (buffer B), 250 mM ammonium acetate/5% ACN/0.012% HFBA/0.5% acetic acid (buffer C), and 1.5 M ammonium acetate/5% ACN/0.012% HFBA/0.5% acetic acid (buffer D). The first 116 min step consisted of a 75 min gradient from 0 to 40% buffer B followed by a 10 min gradient from 40 to 80% buffer B, a 6 min hold at 80% buffer B and a min equilibration step with 100% buffer A. The next 5 steps (146 min each) were characterized by the following profile: 5 min 100% buffer A, 5 min gradient from 0 to x % buffer C, 5 min 100% buffer A, 30 min gradient from 0 to 10% buffer B, 55 min gradient from 10 to 35% buffer B, 20 min gradient from 35 to 50% buffer B, 10 min gradient from 50 to 80% buffer B, a 6 min hold at 80% buffer B, and a 10 min equilibration step with 100% buffer A. The buffer C percentages (x) in steps 2-6 were as follows: 20, 40, 60, 80, and 90%. The 30 min gradient from 0 to 10% buffer B, which is the first linear elution step from the reversed-phase material, was needed in order to sufficiently separate peptide elution from the elution of a major contaminant (m/z=945) which otherwise would have led to the loss of the more hydrophilic peptide peaks. Step 7 consisted of the following profile: 5 min 100% buffer A, 20 min 100% buffer C, 5 min gradient from 0 to 10% buffer B, 35 min gradient from 10 to 35% buffer B, 50 min gradient from 35 to 50% buffer B, 10 min gradient from 50 to 80% buffer B, a 5 min hold at 80% buffer B and a 10 min equilibration step with 100% buffer A. Step 8 was identical to step 7 with the exception of using buffer D instead of buffer C.

Ion Trap MS/MS Mass Spectrometry

The HPLC column was directly coupled to a Finnigan LCQ Deca XP Plus ion trap mass spectrometer (Thermo Finnigan, San Jose, USA) equipped with a nano-LC electrospray ionization source. Mass spectrometry in the MS/MS mode was performed according to the manufacturer's protocol. Peptides were identified by the SEQUEST algorithm (U.S. Pat. Nos. 6,017,693 and 5,538,897).

MALDI-TOF Mass Spectrometry

Peptides spotted onto an Anchor Chip plate were co-cristallized with matrix (5 mg/ml; α-cyano-4-hydroxy-cinnamic acid (Merck, Darmstadt, Germany), 50% acetonitrile, 0.1% trifluoroacetic acid). For qualitative analysis of the whole peptide repertoire, samples were analyzed on an Ultraflex MALDI-TOF mass spectrometer (Bruker, Bremen, Germany), according to the manufacturer's protocol.

Sequence Identification by SEQUEST and Differential Dataset Analysis.

MS/MS fragmentation data were analyzed with the software SEQUEST (Thermo Finnigan, San Jose, USA). From an in-house protein database, which was created based on the public databases Swiss-Prot and TrEMBL, SEQUEST extracted for each spectrum all peptide sequences that corresponded to the molecular mass of the parent ion and measured the degree of similarity between the experimental spectrum and the theoretical, in silico generated, spectrum. Only the top-scoring candidate sequence was listed.

The peptide sequences derived from the SEQUEST analysis and their accompanying information on mass accuracy, scoring parameters and peptide origin were stored in an appropriately designed relational database and further processed. Certain constraints were enforced in order to guarantee the storage of only significant sequences with satisfying SEQUEST scores. The two most important constraints were: (i) keep only those sequences that have a cross correlation coefficient (CC) higher than a certain value and (ii) from the remaining sequences keep those ones which have a predefined delta cross correlation coefficient (ΔCC). For both criteria the minimum chosen values are based on empirical knowledge of interpreting SEQUEST results.

A dataset was defined as the sum of data from a particular set of spectra. The design of database and software allowed queries on a single dataset as well as comparisons of multiple datasets. Such a database and software design enables comparative sample analysis, which is not provided by SEQUEST. For instance, possible queries on a single dataset could provide information on the score distribution among the stored spectra, on the existence of further sequence length variants or common subsequences, or on the protein origin of peptide sequences. Since the occurrence of truncation variants of the same epitope is a general characteristic of class II MHC-bound peptides, the existence of length variants in a dataset provides additional strong evidence for the presence of an epitope in a set of spectra.

The most important feature in the analysis of multiple datasets is the possibility to extract a common subset of sequences that satisfies a given criterion. Such a criterion could be based on sequence similarity, e.g., within all sequences of a collection of datasets, those sequences were selected that had at least one subsequence in common with any other sequence. Such comparisons across different datasets constitute the differential approach (RA samples versus control samples) and thereby optimize the search for candidate RA marker peptides.

The pairwise similarity scores between sequences were calculated by a software routine, which is an implementation of a standard string-comparison algorithm. Subsequently, these scores were used to group closely-related sequences (sequences sharing a common subsequence) in well-separated clusters by an additionally developed software routine, which is based on a well-established algorithm (hierarchical clustering, UPGMA).

The generated clusters (e.g. of peptide truncation variants) were then used to identify closely-related sequences across different datasets.

Overall, the data evaluation software provided the ability to perform swiftly and reproducibly the following:
Select from the sequence output generated by SEQUEST those sequences that satisfy reliable empirical criteria.
Store the data in a database appropriately designed for the discovery process at hand.
Extract information about the sequence content of each stored dataset. This information is valuable in assessing the importance of individual sequences within the given dataset and, consequently, across multiple datasets.
Provide, by virtue of the multiple dataset comparisons, a tool that realizes the differential approach, namely the study of the actual sequence content of one sample versus other(s).

Purification of HLA-DR Molecules

HLA-DR molecules were purified from 10¹⁰ EBV-transformed B cell lines or T2-transfectants by affinity chromatography, using anti-DR monoclonal antibody L243, as previously described (Kropshofer H. et al., PNAS 92 (1995) 8313-8317).

In Vitro Peptide Binding Assay

HA(307-319), PKYVKQNTLKLAT, is an immunodominant epitope from influenza virus hemagglutinin that binds well to HLA-DR4 molecules and was used as a reporter peptide in an in vitro peptide binding assay (Rothbard, J. B. et al., Cell (1988) 52:515-523).

Purified detergent-solubilized HLA-DR4 molecules (200 nM) were co-incubated with biotinylated HA(307-319) peptide (200 nM) and graded amounts of competitor peptide (100 nM-10 μM) for 24 hrs at 37° C. in binding buffer (50 nM sodium phosphate, 50 mM sodium citrate, pH 4.8, 0.1% Zwittergent 3-12) in a total volume of 50 μl. The competitor peptides were derived from the candidate RA antigens identified in this study and purchased as synthetic peptides from Medprobe (Lund, Sweden).

3×10 μl were then diluted 10-fold in PBS containing 0.05% Tween-20 and 1% BSA and incubated for 2 hrs in a microtiterplate (Nalge Nunc), which has been coated over night with anti-DR monoclonal antibody L243. Afterwards the samples were developed by incubation with 0.1 μg/ml EU-labeled streptavidin (Wallay Oy, Turku, Finland) for 45 min according to the manufacturer's protocol. After intensive washing with 0.05% Tween-20 in PBS, Europium fluorescence was measured with a time-resolved fluorometer (VICTOR 1420, Wallac/Perkin Elimer Life Sciences) to quantify the binding of biotinylated HA(307-319) peptide to HLA-DR4 molecules (Arndt, S. O. et al., EMBO J. 19 (2000) 1241-1251).

### Example 1

In this example, the technique mentioned in FIG. 1 was used to identify novel HLA-DR-associated peptide markers derived from serum and synovial fluid of patients with non-erosive RA.

6×10⁶ immature dendritic cells were pulsed with either 1 ml serum (5 samples) or 0.6 ml synovial fluid (2 samples) of patients with non-erosive RA and cultured for 24 hrs in the presence of 10 ng/ml TNFα. As a control, 6×10⁶ dendritic cells were cultured in the presence of TNFα (10 ng/ml) without adding serum but 1 ml of PBS. In an additional experiment 6×10⁶ dendritic cells were pulsed with 1 ml serum from 2 healthy test persons and cultured for 24 hrs in the presence of TNFα (10 ng/ml).

Dendritic cells were lysed in detergent TX-100 and HLA-DR molecules were isolated using mAb L243. HLA-DR-associated peptides were eluted with 0.1% TFA and analyzed by high-throughput 2D-LC-MS/MS technology. Peptide identification was achieved by using the SEQUEST algorithm. The peptide sequences derived from the SEQUEST analysis and accompanying information on mass accuracy, scoring parameters and peptide origin were stored in a database and further processed.

The peptide sequences identified from unpulsed DCs (control 1) and from DCs pulsed with the serum of healthy test persons (control 2) were compared with the peptide sequences identified from DCs pulsed with the serum of non-erosive RA patients. Among the RA-specific sequences, only those peptides were selected for further evaluation that re-occurred in at least three of five non-erosive RA samples.

In each serum sample roughly 600±150 individual peptide sequences (cross correlation coefficient CC>3.0 and ΔCC>0.15) were identified. In the synovia samples the number of individual peptide sequences was slightly smaller (400±30). Approximately 80-85% of the peptides found in RA samples were also identified in control samples, underlining the high reproducibility of the analysis. In the majority of cases, several length variants of the same epitope could be identified which is a typical characteristic of class II MHC-bound antigens and supports the validity of the results (Jones, E. Y., Curr Opin Immunol 9 (1997) 75-79). Further confidence in the quality of the data relies on the fact that several of the identified peptides or proteins have already been described in the context of MHC class II molecules: epitopes derived from ubiquitous proteins like Hsp70, enolase, annexin II, cathepsin C or collagen II, as well as from MHC molecules (HLA-A, -B, -C, -E, -G, and β₂-microglobulin) and CLIP (Chicz, R. M. et al., J Exp Med 178 (1993) 27-47; Sinigaglia, F. & Hammer, J., Curr Opin Immunol 6 (1994) 52-56; Arnold-Schild, D. et al., J Imnunol 162 (1999) 3757-3760; Vogt, A. B. & Kropshofer, H., Trends Biochem Sci 4 (1999) 150-154) were frequently detected.

RA-specific peptide sequences were further validated with regard to binding to the RA susceptibility allele DRB1*0401 by using the TEPITOPE software (Hammer, J. et al., Adv Immunol 66 (1997) 67-100). This software provides means for the qualitative and quantitative prediction of T cell epitopes.

The output of the study consists of an epitope that occurred, apart from one exception, only in non-erosive RA samples (Table 1).

Interferon-Gamma-Inducible Lysosomal Thiol Reductase

A very interesting epitope which was identified in 3 out of 7 non-erosive RA samples from serum and synovia is derived from the interferon-gamma-inducible lysosomal thiol reductase (GILT): the 16-mer GILT (192-207) with the amino acid sequence of SEQ ID NO: 3 (Table 1). Further length variants in three other samples support the relevance of this epitope (Table 1): the 14-mer GILT (192-205; SEQ ID NO: 1) and the 17-mer GILT (192-208; SEQ ID NO: 2).

As judged from the shortest length variant, GILT (192-205), the epitope contains a suitable binding motif, with regard to binding to the RA susceptibility allele DRB1*0401: 196M serves as a P1 anchor, 199M as a P4 anchor and 201A as a P6 anchor. According to TEPITOPE scoring, the epitope has a binding score (threshold value) of 1% which is similar to the binding score of an epitope from influenza haemagglutinin (307-319) that was shown to be a strong DRB1*0401 binder (Table 1) (Rothbard, J. B. et al., Cell 52 (1988) 515-523).

GILT is constitutively expressed in antigen-presenting cells, such as dendritic cells, macrophages and B cells, and facilitates unfolding of endocytosed antigens in MHC class II-containing compartments (MIIC) by enzymatically reducing disulfide bonds (Phan, U. T. et al., J Biol Chem 275 (2000) 25907-25914). Direct binding of GILT to HLA-DR molecules has been reported for B cells (Arunachalam, B. et al., J Immunol 160 (1998) 5797-5806). A rather long second epitope of GILT was found to bind to HLA-DR3 molecules: the 22-mer GILT (38-59) having the amino acid sequence SPLQALDFFGNGPPVNYKTGNL (Chicz, R. M. et al., J Exp Med 178 (1993) 27-47).

In addition to GILT (192-207), another epitope of the same protein was identified in several RA samples, but also in control samples: GILT (210-227) with the amino acid sequence QPPHEYVPWVTVNGKPLE. This epitope was accompanied by 3 other length variants: the 16-mer GILT (210-225), the 17-mer GILT (210-226) and the 19-mer GILT (210-228).

As indicated by its name, GILT expression can be induced by the pro-inflammatory cytokine interferon gamma (IFN-γ) in various types of cells, including macrophages, endothelial cells and fibroblasts (Luster, A. D. et al., J Biol Chem 263 (1988) 12036-12043). As IFN-γ is known to be present in inflamed joints of RA patients, GILT could become over-expressed in synovia and serum and, hence, could be taken up by DCs as an exogenous antigen. GILT (192-207) may be derived from exogenous GILT. The other GILT epitope, which is also present in the control samples, may be derived from endogenous GILT, expressed by DCs. Alternatively, both GILT (192-207) and GILT (210-227) may be derived from endogenous GILT, in case that GILT processing and GILT-derived epitope presentation by DCs were critically altered upon contact with RA-associated material.

The identified epitope of Interferon-gamma-inducible lysosomal thiol reductase GILT (192-205) which has been described already in detail, was further analysed in an in vitro binding assay using synthetic GILT (192-205) peptide and purified HLA-DR4 molecules (FIG. 3): In agreement with TEPITOPE scoring, the peptide was shown to bind to HLA-DR4 with high affinity, comparable to the viral HA(307-319) peptide.

Integrin Beta-2

The extended analysis revealed the presence of an epitope which was identified in two out of seven non-erosive RA samples from serum and synovia and which is derived from the beta subunit of Integrin (ITB2): the 17-mer ITB2 (315-331; SEQ. ID NO: 58) with the amino acid sequence NIQPIFAVTSRMVKTYE (Table 1). One length variant was found: the 19-mer ITB2 (313-331; SEQ. ID NO: 59) supporting the validity of the identified epitope (Table 1).

The peptide sequence contains a moderate HLA-DRB1*0401 binding motif with 316I and 319I serving as P1 and P4 anchor, respectively (TEPITOPE binding score: 2%).

Integrins are a family of cell surface receptors that play important roles in embryogenesis, wound healing, immune responses, and cell adhesion. ITB2 is a heterodimeric receptor for intercellular and vascular adhesion molecules (ICAMs and VCAMs) and exclusively expressed on leukocytes. ICAMs and VCAMs are members of the immunoglobulin superfamily that has a central function in humoral and cell-mediated immune responses. Many cytokines, such as interferon-γ, IL-1 and TNFα, which are upregulated in inflammatory diseases like RA, induce expression of ICAMs on the surface of endothelial cells. It was shown that the expression of ICAM-1 and VCAM-1 is higher in synovial tissue of RA patients as compared to osteoarthritis patients (Furuzawa-Carballeda, J. et al., Scand J Immunol, 50 (1999) 215-222).

Binding of ITB2 and other integrins to ICAMs allows leukocytes to infiltrate into their target tissues, e.g. the sites of inflammation. In order to function in a non-adherent or circulating mode, leukocytes constitutively express ITB2 and other integrins with low ligand-binding capacity. The ITB2/ICAM-1 interaction has become an important strategic target to approach inflammation, autoimmune diseases and cancer (Yusuf-Makagiansar, H. et al., Med Res Rev 22 (2002) 146-167), hence these findings strongly support the validity of the identified ITB2 epitope as a candidate marker for RA.

Phosphatidylinositol-4,5-bisphosphate 3-kinase

Another epitope with a moderate HLA-DRB1*0401 binding motif was identified in two non-erosive serum samples: the 17-mer PI3K (792-808; SEQ. ID NO: 60) with the amino acid sequence NKVFGEDSVGVIFKNGD derived from Phosphatidylinositol 3-kinase (PI3K) (Table 1). In this peptide 794V could serve as a hydrophobic P1 anchor, 797E as a negatively charged P4 anchor, and 799S as a typical DR4-P6 anchor (TEPITOPE binding score: 3%).

PI3K is ubiquitously expressed in many cell types and phosphorylates lipids, predominantly phosphatidylinositol-4,5-bisphosphate. The protein has emerged as a key signal transducer for survival factor receptors, including growth factors, cytokines, and integrins (reviewed by Toker, A. & Cantley, L., Nature 387 (1997) 673-676). In addition, PI3K plays an important role in the signaling pathway of Toll-like receptors (TLRs) that recognize a variety of microbial products, collectively termed pathogen-associated molecular patterns (PAMPs) (Fukao, T. & Koyasu, S., Trends Immunol 24 (2003) 358-363). Stimulation through TLRs by PAMPs, such as lipopolysaccharide (LPS) (endotoxin) triggers production of various cytokines, including IL-12, which is a key cytokine in TLR-mediated Th1 responses. It was shown that PI3K is an endogenous suppressor of TLR-mediated IL-12 production and limits excessive Th1 polarization. Thus PI3K was suggested to be a negative regulator of innate immune responses in order to prevent prolonged activation of innate immunity harmful to the host. The knowledge that autoimmune responses in RA rely on Th1 cytokines, ultimately links PI3K with RA. Although no specific pathogen has been consistently linked to the development of RA, it is believed that RA develops in two stages in which an initial response induced by foreign antigens, subsequently develops into a self-sustaining autoimmune response (Klinman, D., Arthritis Rheum 48 (2003) 590-593).

Urokinase-Type Plasminogen Activator

Another epitope which was only found in non-erosive RA samples was derived from a urokinase-type plasminogen activator (uPA): the 16-mer uPA (328-343; SEQ. ID NO: 61) with the amino acid sequence YPEQLKMTVVKLISHR (Table 1). With regard to HLA-DRB1*0401 binding, the sequence reveals a moderate binding motif 332L, 335T and 337V are putative P1, P4 and P6 anchors, respectively (TEPITOPE binding score: 2%).

Plasminogen activators (PAs) are highly specific serine proteases generating plasmin from plasminogen. Two types of PAs, a urokinase-type (uPA) and a tissue-type (tPA) have been identified in mammals. The activities of PAs are controlled by natural inhibitors (PAIs). Plasmin is involved in inflammatory reactions by inducing cytokines such as TGFβ, and in cartilage and fibrin degradation. Evidences for ongoing coagulation. within the rheumatoid joint together with an enhancement of uPA synthesis and activation in arthritic joints have led to the hypothesis that the PA/plasmin system is associated with the clinical severity of arthritis (reviewed by Busso, N. & Hamilton, J. A., Arthritis Rheum 46 (2002) 2268-2279). Based on in vitro studies, there are several cell types present in the inflamed joints of RA patients, in particular monocytes and synovial fibroblasts which can express PAs and PAIs and which therefore could contribute to the in vivo levels. The activity of PAs (i.e. uPA) is stimulated by cytokines, such as IL-1 and TNFα, which are known to be highly upregulated in serum as well as in synovial fluid of RA patients. These lines of evidence render uPA (328-343) a putative peptide marker for RA.

Immunglobulin Heavy Chain V-III Region (V_{H}26)

One of the identified epitopes that display a very strong binding motif with regard to HLA-DRB1*0401 binding, is derived from the V_{H}26 gene segment of the immunglobulin heavy chain: the 16-mer V_{H}26 (95-110; SEQ. ID NO: 62) with the amino acid sequence KNTLYLQMNSLRAEDT (Table 1). The high TEPITOPE binding score (1%) is substantiated by 99Y functioning as a very potent P1 anchor, 102M as a moderate P4 anchor and 104S as a typical HLA-DR4P6 anchor.

Immunglobulins (Ig) are responsible for antigen binding and stimulate further immune reactions, e.g. by binding to isotope-specific Fc receptors. Interestingly, the human V_{H}26 gene segment appears to encode a high-avidity synovial rheumatoid factor and thus seems to have an impact on RA progression (Wong, A. et al., Autoimmunity 20 (1995) 191-199). The fact that the identified epitope V_{H}26 (95-110) was found in the serum of two seropositive RA patients (Table 1) correlates with the above observation.

DJ-1 Protein

In two non-erosive RA samples our continued studies revealed the presence of an epitope which is derived from a protein termed DJ-1: the 16-mer DJ-1 (135-150; SEQ. ID NO: 63) with the amino acid sequence NGGHYTYSENRVEKDG (Table 1). According to TEPITOPE scoring the peptide contains a rather weak HLA-DRB1*0401 binding motif with 139Y serving as a P1 anchor, 142S as a P4 anchor and 144N as a possible P6 anchor (binding score: 8%).

DJ-1 belongs to the ThiJ/PfpI protein family whose members are evolutionary distributed from Archaea to Eukarya. ThiJ/PfpI proteins share a conserved ThiJ domain that is structurally related to the type I glutamine amidotransferase domain (Lee, S. J. et al., J Biol Chem 25 (2003) Epub ahead of print).

DJ-1, which is preferentially expressed in testis and moderately in other tissues, was first identified as a novel candidate of the oncogene product that transformed mouse NIH3T3 cells in cooperation with ras. In the mean time additional physiological roles of DJ-1 were revealed including a strong correlation to Parkinson disease and sperm fertilization. DJ-1 seems to have multiple functions—here we provide the first indication for an association of DJ-1 with RA.

### Example 2

In this example, the same technology was used that has been described in detail in example 1. Serum (6 samples) and synovial fluid (2 samples) of patients with diagnosed erosive RA were utilized in this case to identify candidate markers specific for erosive RA.

The peptide sequences found in the erosive RA samples were compared with the sequences identified in unpulsed DCs (control 1) and in DCs pulsed with the serum of healthy test persons (control 2). Among the RA-specific sequences, only those peptides were selected for further evaluation that re-occurred in at least three of six erosive RA samples.

In this study one epitope was discovered which occurred, apart from one exception, only in erosive RA samples.

Apolipoprotein B-100

The epitope which was mainly found in erosive RA sera (4 out of 8 erosive RA samples) is derived from apolipoprotein B-100: the 16-mer ApoB (2877-28.92) with the amino acid sequence of SEQ ID NO: 4 (Table 2). In addition a length variant of the same epitope was identified (Table 2): the 17-mer ApoB (2877-2893; SEQ ID NO: 5). The following DRB1*0401 binding motif can be predicted: 2881L as a PI anchor, 2884D as a P4 anchor and 2886N as a P6 anchor (binding score 3%).

In an earlier study on EBV-B cells, the epitope ApoB (2885-2900), which partly overlaps with the epitope described here, has been found in the context of HLA-DR4 (Chicz, R. M. et al., J Exp Med 178 (1993) 27-47).

Apolipoprotein B-100 is a constituent of very low-density lipoproteins (VLDL) and low-density lipoproteins (LDL) and functions as a recognition signal for the cellular binding and internalization of LDL particles by the ApoB/E receptor (Yang, C. Y. et al., Nature 323 (1986) 738-742). Interestingly, an increased ratio of LDL cholesterol to HDL cholesterol was observed among newly diagnosed RA patients (Park, Y. B. et al., J Rheumatol 26 (1999) 1701-1704). The adverse lipid profile in active RA could be improved by treating RA patients with DMARDs without the use of lipid-lowering agents (Park, Y. B. et al., Am J Med 113 (2002) 188-193). Since an increased cardiovascular mortality among patients with chronic inflammatory diseases, such as RA, is well documented (Symmons, D. P. et al., J Rheumatol 25 (1998) 1072-1077) it was suggested that local inflammation in RA leads to altered blood lipid levels, thereby increasing the risk of atherosclerosis. The question whether components of the lipoprotein metabolism are causal for pathogenesis or merely affected by ongoing immune reactions during RA development cannot be answered yet. However, the observation of adverse lipid profiles in RA patients supports the validity of the presented ApoB epitope as a serum-derived RA candidate marker.

The length variant ApoB (2877-2892), but not ApoB (2877-2893), has been identified in samples of two healthy controls (Table 2). Since Apolipoprotein B constitutes 1% of all plasma proteins, the presence of ApoB epitopes in healthy control samples is not surprising. The results suggest that only the length variant ApoB (2877-2893; SEQ ID NO: 5) is specific for erosive RA.

26S Proteasome Non-ATPase Regulatory Subunit 8

An epitope which was quite frequently identified in mostly erosive RA samples both from serum and synovia, is derived from the regulatory subunit 8 of the 26S proteasome (PSMD8): the 15-mer PSMD8 (218-232; SEQ. ID NO: 64) with the amino acid sequence GPNNYYSFASQQQKP (Table 2). Three additional length variants were identified: the 16-mer PSMD8 (218-233; SEQ. ID NO: 65), the 17-mer PSMD8 (218-234; SEQ. ID NO: 66) and the 18-mer PSMD8 (218-234; SEQ. ID NO: 67) (Table 2). The presence of length variants supports the validity of the identified epitope as a class II MHC-derived antigenic peptide. The peptide displays a moderate binding motif with respect to DRB1*0401 binding (TEPITOPE binding score: 3%). Binding to HLA-DR4 could be confirmed in an in vitro binding assay using synthetic PSMD8 (218-233) peptide and purified HLA-DR4 molecules (FIG. 3): According to its IC₅₀ value against the reporter peptide HA(308-319), PSMD8 (218-233; SEQ. ID NO: 65) binds to HLA-DR4 with a moderate affinity, confirming the TEPITOPE prediction. The 15-mer PSMD8 (218-232; SEQ. ID NO: 64) was identified once in an unpulsed control sample.

The proteasome accounts for about 1% of the cytoplasmic protein pool and is involved in ATP-dependent degradation of ubiquitinated proteins. Moreover, the proteasome is responsible for the processing of several transcription factors (i.e. nuclear factor-KB), for cell cycle control and the generation of MHC class I restricted antigens. The regulatory subunits of the proteasome are important for the selectivity of protein degradation. The non-ATPase regulatory subunit 8, in particular, is known to be necessary for the activation of the cell division control protein 28 (Cdc28), an essential cell cycle regulator in yeast.

Importantly, strongly increased levels of circulating proteasomes (cProteasomes) were detected in serum samples obtained from patients with different systemic autoimmune diseases, including RA (Egerer, K. et al., J Reumatol 29 (2002) 2045-2052). There appears to be a close correlation between the levels of cProteasome and disease activity and concentrations of C-reactive protein in patients with severe RA cProteasome is discussed to trigger subsequent immune responses, which would indicate an antigen-driven mechanism. The concentration of released Proteasome antigen seems to reflect the magnitude of cellular damage in autoimmune diseases. From their findings Egerer and coworkers concluded that cProteasomes could represent a novel marker for disease severity in autoimmune processes. Since the epitope PSMD8 (218-232; SEQ. ID NO: 64) was mainly identified in erosive RA samples our analysis supports this conclusion.

Interleukin-1 Receptor

Another candidate marker for erosive RA is the Interleukin-1 receptor (IL-1R) which was identified in two erosive serum samples via its peptide IL-1R (79-94; SEQ. ID NO: 68) with the amino acid sequence EKLWFVPAKVEDSGHY (Table 2). IL-1R (79-94; SEQ. ID NO: 68), with 83F as a P1 anchor, 86A as a P4 anchor and 88V as a P6 anchor, contains a strong binding motif with regard to binding to the RA susceptibility allele DRB1*0401 (TEPITOPE binding score: 1%). In an in vitro binding assay the synthetic peptide was shown to bind to HLA-DR4 molecules with high affinity (FIG. 3), similar to HA(309-319) peptide.

Interleukin-1 (IL-1) is a proinflammatory cytokine and implicated in a variety of infectious immune responses as well as in RA and other inflammatory diseases (Dinarello, C., Blood 87 (1996) 2095-2147). It binds to its respective receptor which functions as a signal transducer to trigger cell proliferation or to stimulate protein synthesis. Increased IL-1 production has been observed in RA patients and plays a pivotal role in its clinical manifestations (Dayer, J. M., Rheumatology 42 (2003) ii3-ii19). IL-1 is viewed as a key mediator in RA through activation of macrophages and T- and B-lymphocytes. In addition, IL-1 contributes to inflammation by inducing the expression of cell-adhesion molecules, other cytokines and chemokines. Furthermore IL-1 is also pivotal in the destruction of bone and cartilage in RA by stimulating the production of matrix metalloproteinases. Thus, the IL-1/IL-1R complex is a first class target for anti-inflammatory therapeutics. The use of a recombinant human IL-1 receptor antagonist (IL-IRA) has been approved for the treatment of RA patients.

Fibromodulin

Three erosive RA samples gave rise to an epitope derived from the secreted matrix protein fibromodulin (FM): the 13-mer FM (178-190; SEQ. ID NO: 70) with the amino acid sequence LRELHLDHNQISR (Table 2). Furthermore, the 14-mer FM (177-190; SEQ. ID NO: 69) was identified, which was also found once in an unpulsed control experiment. The epitope depicts a strong DRB1*0401 binding motif with 181L serving as a P1 anchor, 184D as a P4 anchor and 186N as a P6 anchor (TEPITOPE binding score: 1%).

Fibromodulin belongs to a family of small leucine-rich proteoglycans (SLRPs) that bind to TGFβs and collagens and other extracellular matrix molecules. In vitro, SLRPs were shown to regulate collagen fibrillogenesis, a process essential in development, tissue repair and metastasis. To better understand the function of SLRPs in vivo, SLRP-deficient mice were generated and shown to develop a wide array of diseases (e.g. osteoporosis and osteoarthritis), most of them resulting primarily from an abnormal collagen fibrillogenesis (Ameye, L. & Young, M. F., Glycobiology 12 (2002) 107R-116R). Since collagen formation and degradation is highly enhanced in the inflamed joints of RA patients, an increased level of fibromodulin can be envisaged. In accord with this consideration is the fact that the identified FM epitope was primarily found in RA samples of synovial origin (3 out of 4 synovia samples).

GM-CSF/IL-3/IL-5 Receptor

In 5 out of 8 samples from erosive RA patients an epitope from the β-chain of the multiple cytokine receptor CYRB could be identified: the 15-mer CYRB (359-373; SEQ. ID NO: 71) with the amino acid sequence ETMKMRYEHIDHTFE and its length variant, the 17-mer CYRB (359-375; SEQ. ID NO: 72) (Table 2). In an in vitro binding assay the synthetic CYRB (359-375) peptide was shown to bind to HLA-DR4 molecules with moderate affinity (FIG. 3). This is in agreement with TEPITOPE which predicted a moderate peptide binding motif with respect to DRB1*0401 binding (TEPITOPE binding score: 3%). The epitope was clearly overrepresented in the RA samples since it was identified in the serum of only one healthy test person.

The GM-CSF/IL-3/IL-5 receptor is a type I membrane protein and differentially expressed throughout the hematopoietic system (reviewed by Geijsen, N. et al., Cytokine Growth Factor Rev 12 (2001) 19-25). Its ligands, IL-3 and GM-CSF, are secreted by CD4+ T cells and important stimuli for the formation of dendritic cells originating from progenitor cells in the bone marrow. The critical role of dendritic cells in the initiation and development of an immune response suggests that they play a key role in the development of autoimmune inflammatory disorders, such as RA, by transporting autoantigen to the draining lymph node where DCs encounter and prime naïve T cells. The activity of GM-CSP has been linked to proinflammatory effects in RA supporting the identified receptor epitope CYRB (359-373; SEQ. ID NO: 71) as a putative candidate peptide marker for the diagnosis of RA.

Sorting Nexin 3

Another epitope which appears to be indicative for erosive RA is derived from a protein termed sorting nexin 3 (SNX3): the 16-mer SNX3 (142-157; SEQ. ID NO: 73) having the amino acid sequence HMFLQDEIIDKSYTPS (Table 2). In this epitope 144F, 147D and 149I could serve as P1, P4 and P6 anchors, respectively, in the peptide-binding groove of the RA susceptibility allele DRB1*0401 (TEPITOPE binding score: 2%).

Sorting nexins are a diverse group of cellular trafficking proteins that share a common phospholipid-binding motif (reviewed by Worby, C A. & Dixon, J. E., Nat Rev Mol Cell Biol 3 (2002) 919-31). The ability of these proteins to bind specific phospholipids, as well as their propensity to form protein-protein complexes, points to the involvement of these proteins in membrane trafficking and protein sorting. Sorting nexin 3 in particular is present in the cytosol and in endosomes and appears to be involved in membrane trafficking from early to recycling endosomes. Whether sorting nexin 3 plays a role in RA, e.g. by influencing antigen presentation routes, remains unknown at the moment. The epitope SNX3 (142-157; SEQ. ID NO: 73) identified in this study suggests a link between sorting nexins and autoimmunity.

### Example 3

All peptide sequences identified in examples 1 and 2 from non-erosive and erosive RA samples were used in this example to search for common markers relevant for both RA types. The RA-specific sequences were again compared with peptide sequences of the control samples (unpulsed DCs and DCs pulsed with the serum of two healthy test persons) and only those peptides were selected for further evaluation that re-occurred in at least three of altogether fifteen RA samples (erosive and non-erosive RA).

Inter-Alpha-Trypsin Inhibitor

Ten out of eleven serum samples (erosive & non-erosive RA) gave rise to an epitope derived from the heavy chain H4 of the inter-alpha-trypsin inhibitor: ITIH4 (271-287) with the amino acid sequence of SEQ ID NO: 8 (Table 3). Apart from this major length variant of the ITIH4 epitope, six length variants of the same ITIH4 epitope could be identified (Table 3): the 19-mer ITIH4 (271-289; SEQ ID NO: 6), the 18-mer ITIH4 (271-288; SEQ ID NO: 7), the 16-mer ITIH4 (274-289; SEQ ID NO: 12), the 15-mer ITIH4 (273-287; SEQ ID NO: 10), the 15-mer (274-288; SEQ ID NO: 11) and the 14-mer ITIH4 (274-287; SEQ ID NO: 9).

As judged from the shortest length variant, ITIH4 (274-287), the epitope contains a very strong binding motif, with regard to binding to the RA susceptibility allele DRB1*0401: 277F serves as a P1 anchor, 280D as a P4 anchor and 282S as a P6 anchor (binding score: 1%).

ITIH4 belongs to the Inter-alpha-inhibitor (IαI) family which is a group of serum protease inhibitors that bind to hyaluronic acid (HA) and appear to be involved in acute-phase reactions (Salier, J. P. et al., Biochemical Journal 315 (1996) 1-9).

HA is a polysaccharide found in all tissues of the body, in particular, in loose connective tissue, e.g. joint fluid (Evered, D. & Whelan, J. eds., The Biology of Hyaluronan, John Wiley & Sons (1989)). HA has an important structural function in cartilage and other tissues where it stabilizes the extracellular matrix by forming aggregates with proteoglycans. It has also been assigned important biological functions by regulating cellular activities via binding to cell surface proteins, such as CD44 and ICAM-1 (Knudson, C. B. & Knudson, W., FASEB J 7 (1993) 1233-1241; Hall, C. L. et al., J Cell Biol 126 (1994) 575-588). RA is accompanied by a large increase in total HA in the joint fluid as well as in the serum, suggesting that circulating HA originates from rheumatoid joints (Engström-Laurent, A. et al., Scand J Clin Lab Invest 45 (1985) 497-504).

Complexes of HA and some IαI family members were observed in large amounts in the synovial fluid of RA patients (Jessen, T. E. et al., Biological Chemistry Hoppe-Seyler 375 (1994) 521-526). The role of the IαI-HA complex in inflammatory reactions might be to modify the CD44-HA interaction that mediates leukocyte activation and invasion (Isacke, C. M. & Yarwood, H., Int J Biochem Cell Biol 34 (2002) 718-721). Additionally, synovial fluid of RA patients contains elevated levels of TSG-6, an anti-inflammatory glycoprotein and a member of the hyaladherin family of HA-binding proteins (Wisniewski, H. G. et al., J Immunol 151 (1993) 6593-6601). It has been shown that a complex of TSG-6 with IαI family members inhibits the activity of plasmin, a central molecule in the activation of inflammation-associated enzymes (Wisniewski, H. G. et al., J Immunol 156 (1996) 1609-1615). A regulation of plasmin activity by several acute-phase plasma proteins, namely TSG-6 and IαI family members, may prove to be important in RA, given the high contents of HA, TSG-6 and IαI family members in synovial fluid of inflamed joints.

This evidence, together with the identification of multiple length variants of the same epitope and a strong HLA-DR4 binding motif, convincingly support the validity of the presented ITIH4 epitope as a serum-derived RA candidate marker.

Complement C4

In eight out of eleven RA sera (erosive and non-erosive) tested, another dominant epitope was identified which is derived from complement C4: the 15-mer C4 (1697-1711) with the amino acid sequence of SEQ ID NO: 13 (Table 3). Five additional length variants of the same epitope could be found (Table 3): the 12-mer C4 (1697-1708; SEQ ID NO: 18), the 13-mer C4 (1698-1710; SEQ ID NO: 17), the 14-mer C4 (1697-1710; SEQ ID NO: 15), the 16-mer C4 (1697-1712; SEQ ID NO: 14) and the 18-mer C4 (1697-1714; SEQ ID NO: 16). Moreover, the presented epitope displays a very strong DRB1*0401 binding motif 1700Y as P1 anchor, 1704D as P2 anchor and 1706N as P6 anchor (binding score: 1%).

C4 which constitutes approximately 0.5% of plasma protein mass plays a critical role in the triggering of the central pathway of the complement system. The protein is synthesized as a single-chain precursor and, prior to secretion, is enzymatically cleaved to form a trimer of non-identical α-, β-, and γ-chains. The identified epitope C4 (1697-1711) is located at the very C-terminus of the C4 γ-chain. The C4 α-chain is further proteolytically degraded by activated C1 to form the C4a anaphylatoxin, which is a mediator of local inflammatory processes (Moon, K. E. et al., J Biol Chem 256 (1981) 8685-8692).

In general, the complement cascade is involved in the induction and progression of inflammatory reactions and is a major defense system against various pathogenic agents, including bacteria, viruses and other antigens (Morgan, B. P., Methods Mol Biol 150 (2000) 1-13). Inappropriate activation, however, can lead to tissue damage and manifestation of disease (Speth, C. et al., Wien Klin Wochenschr 111 (1999) 378-391).

Activation of the complement system has been repeatedly implicated in the pathogenesis of RA, based on studies showing increased levels of complement metabolites, including C4 and C4a, in plasma, synovial fluid and synovial tissue of RA patients (Neumann, E. et al., Arthritis Rheum 46 (2002) 934-945). In addition collagen-induced arthritis (CIA) in mice is characterized by the presence of complement activation products (Linton, S. M. & Morgan, B. P., Mol Immunol 36 (1999) 905-914). CIA is prevented after treatment with anti-C5 monoclonal antibodies (Wang, Y. et al., PNAS 92 (1995) 8955-8959) or with soluble CR1, an inhibitor of the complement system, delivered by gene therapy (Dreja, H. et al., Arthritis Rheum 43 (2000) 1698-1709). Activation of complement factors in joints is possibly induced by the presence of various immune complexes and it was hypothesized that stimulation of the innate immune system by infectious agents and cytokines may contribute to the initiation of RA (Friese, M. A. et al., Clin Exp Immunol 121 (2000) 406-414).

Two of the six presented C4 epitopes, the 15- and the 18-mer, were also identified in healthy control samples (Table 3) indicating that only some length variants of this C4 epitope are RA-specific, namely the antigenic peptides of SEQ ID NOs: 14, 15, 17, and 18.

Complement C3

Another epitope that was found in erosive and non-erosive RA samples is derived from complement C3 (alpha-chain): the 14-mer C3 (1431-1444) with the amino acid sequence of SEQ ID NO: 21 (Table 3). Six additional length variants of the same epitope were identified in serum (Table 3): the 13-mer C3 (1431-1443; SEQ ID NO: 23), the 14-mer C3 (1429-1442; SEQ ID NO: 74), the 15-mer C3 (1431-1445; SEQ ID NO: 22), the 15-mer C3 (1429-1443; SEQ ID NO: 20), the 17-mer C3 (1427-1443; SEQ ID NO:75) and the 19-mer C3 (1426-1444; SEQ ID NO: 19). As judged from the shortest length variant, C3 (1431-1443), a DRB1*0401 binding motif can be postulated: 1434Y serves as a P1 anchor, 1437D as a P4 anchor and 1439A as a P6 anchor.

In erovise and non-erosive RA samples a further epitope was found, which is derived from complement C3 (beta-chain): the 19 mer C3 (157-175) with the amino acid sequence of SEQ. ID NO: 76 (Table 3). One additional length variant of the same epitope was identified in serum (Table 3): 20-mer C3 (157-176; SEQ. ID NO: 77)

Complement C3 which constitutes about 1-2% of plasma protein mass plays a central role in the activation of the complement system and belongs to the family of the acute-phase proteins. Its processing by C3 convertase to C3a anaphylatoxin and C3b is the central step in both the classical and alternative complement pathways (Barrington, R. et al., Immunol Rev 180 (2001) 5-15). After activation, C3b can bind covalently, via a reactive thiolester, to cell surface carbohydrates or immune aggregates (Isaac, L. & Isenman, D. E., J Biol Chem 267 (1992) 10062-10069). The identified epitope C3 (1431-1444) is located at the C-terminus of C3b.

As already discussed in the context of complement epitope C4 (1697-1711), there is increasing evidence for an important role of components of the complement cascade in the pathophysiology of RA. The result of this study, in which two major epitopes derived from complement C3 and C4 were identified in serum of RA patients, underlines the close link between the activated complement system and pathogenesis of RA. This coincidence makes a strong argument for the validity of the presented C3/C4 epitopes as serum-derived candidate RA markers.

SH3 Domain-Binding Glutamic Acid-Rich-Like Protein 3

Another epitope which was elucidated quite frequently in serum of RA patients (six out of eleven erosive and non-erosive RA samples), is derived from the SH3 domain-binding glutamic acid-rich-like protein 3 (SH3BGRL3): SH3BGRL3 (15-26) with the amino acid sequence of SEQ ID NO: 25 (Table 3). Three length variants of the same epitope were identified (Table 3): the 14-mer SH3BGRL3 (13-26; SEQ ID NO: 26), the 14-mer SH3BGRL3 (15-28; SEQ ID NO: 27) and the 16-mer SH3BGRL3 (13-28; SEQ ID NO: 24). The DRB1*0401 binding motif is: 17I as P1 anchor, 20Q as P4 anchor and 22S as P6 anchor (binding score 4%).

SH3BGRL3 is a small 10 kD protein that belongs to the SH3BGR family. The precise function of the protein is unknown but a role as a modulator of glutaredoxin biological activity is postulated (Mazzocco, M. et al., Biochem Biophys Res Commun 285 (2001) 540-545). So far, SH3BGRL3 has not been described in the context of RA.

Interestingly, the analysis elucidated a second epitope of the same protein, which was highly abundant in all RA and control samples: the 16-mer SH3BGRL3 (29-44) with the amino acid sequence DGKRIQYQLVDISQDN. In addition multiple length variants of the same epitope were found in most samples as well. As judged from the shortest length variant, SH3BGRL3 (31-42), the epitope contains almost similar DRB1*0401 anchor residues compared with SH3BGRL3 (15-26): 33I serves as a P1 anchor, 36Q as a P4 anchor and 38V as a P6 anchor (binding score-2). This similarity is reflected by comparable binding scores.

The presence of this second SH3BGRL3 epitope supports the validity of the SH3BGRL3 (15-26) epitope because both peptides are derived from the same protein, however, only one of them, epitope SH3BGRL3 (15-26), appears to be generated in a RA-specific manner. A similar observation has been described already for GILT in example 1.

Among the four SH3BGRL3 length variants the longest variant, SH3BGRL3 (13-28), was also identified in a healthy control sample (Table 3). However, this particular length variant was found only one time, which indicates a significant enrichment of the SH3BGRL3 epitope in the context of RA.

Interleukin-4 (IL-4) Induced Protein 1

In all the investigated synovial fluids (erosive & non-erosive RA) and in eight out of eleven sera (erosive & non-erosive RA), one highly dominant epitope was identified which is derived from the human homolog of the IL-4 induced protein 1 (Fig1): Fig1 (293-309) with the amino acid sequence of SEQ ID NO: 28 (Table 3). The validity of the epitope was further supported by the presence of additional length variants in several samples (Table 3): the 16-mer Fig1 (293-308; SEQ ID NO: 30) and the 19-mer Fig1 (293-311; SEQ ID NO: 29). Moreover, the amino acid sequence displays a typical DRB1*0401 binding motif: 299V serves as P1 anchor, 302E as P4 anchor and 304S as P6 anchor (binding score 1%).

Two length variants of the same epitope, Fig1 (293-308) and Fig1 (293-309), were identified in one unpulsed sample and in one healthy control sample as well (Table 3). However, the presence of the Fig1 epitope in almost all RA samples but not in all of the control samples tested strongly indicates an enrichment in the context of RA.

The human fig1 gene was first identified in IL-4-stimulated B cell cultures (Chu, C. C. & Paul, W. E., PNAS 94 (1997) 2507-2512). The human fig1 resides on chromosome 19q13.3-19q13.4, a region previously identified to be involved in susceptibility to autoimmune diseases, including SLE, arthritis, multiple sclerosis, and insulin-dependent diabetes mellitus (Becker K. G. et al., PNAS 95 (1998) 9979-9984). Since its expression is largely limited to immune tissues and its regulation is dependent on IL-4, a key modulator of the immune response, fig1 is thus an attractive candidate gene for autoimmune disease susceptibility (Chavan, S. S. et al., Biochim Biophys Acta 1576 (2002) 70-80). The HLA-DR4-restricted presentation of a Fig1 epitope provides the first indication that Fig1 protein is produced and possibly involved in the disease development of RA. The Fig1 polypeptide has not been known as a marker for RA until now, and is considered as an important candidate marker for RA.

Hemopexin

Another RA candidate marker which was frequently identified in serum samples (ten out of eleven samples) and in synovia samples (two out of four samples) (erosive & non-erosive RA) is derived from hemopexin (HPX): HPX (351-367) with the amino acid sequence of SEQ ID NO: 32 (Table 3). Several length variants were found which support the validity of this epitope (Table 3): the 13-mer HPX (351-363; SEQ ID NO: 33), the 14-mer HPX (350-363; SEQ ID NO: 34), the 15-mer HPX (351-365; SEQ ID NO: 35), the 18-mer HPX (351-368; SEQ ID NO: 31) and the 18-mer HPX (350-367; SEQ ID NO: 78). Furthermore, the epitope contains a very strong DRB1*0401 binding motif 355I serves as a P1 anchor, 358D as a P4 anchor and 360V as a P6 anchor (binding score: 1%).

Two length variants of the same epitope, HPX (351-367; SEQ ID NO: 32) and HPX (351-365; SEQ ID NO: 35), could also be identified in healthy control samples (Table 3) indicating that only some length variants are specific for RA, namely the antigenic peptides of SEQ ID NOs. 31, 33, 34 and 78.

HPX is a 60 kD plasma glycoprotein with a high binding affinity to heme (Müller-Eberhard, U., Methods Enzymol 163 (1988) 536-565). It is mainly expressed in the liver, and belongs to the acute-phase proteins the synthesis of which is induced in an inflammatory situation. RA is a chronic inflammatory autoimmune disease and elevated levels of several acute-phase proteins, including C-reactive protein and serum amyloid A, have been reported (Nakamura, R., J Clin Lab Anal 14 (2000) 305-313). HPX is responsive to the cytokines IL-1 and IL-6, which are upregulated in patients suffering from RA (Feldmann, M. & Maini, R.N., Rheumatology 38, Suppl 2 (1999) 3-7).

HPX is the major vehicle for the transportation of heme in the plasma and its principal role is to prevent heme-mediated oxidative stress and loss of heme-bound iron (Tolosano, E. & Altruda, F., DNA Cell Biol 21 (2002) 297-306). It can protect cells against oxidative stress by inducing the expression of intracellular antioxidants such as heme oxygenase, metallothioneins and ferritin. Metallothioneins are cytosolic proteins that are expressed particularly in synovial fibroblasts (Backman, J. T. et al., Virchows Arch 433 (1998) 153-160). There is significant experimental evidence for the presence of oxidative stress in the synovial tissue of RA patients (reviewed in: Schett, G. et al., Arthritis Res 3 (2000) 80-86). Furthermore HPX was reported to promote proliferation of human T lymphocytes (Smith, A. et al., Exp Cell Res 232 (1997) 246-254). These studies render it likely that HPX belongs to the up-regulated proteins in serum and synovia of RA patients, thereby providing a rationale for the relevance of HPX (351-367) as a RA-specific candidate marker.

Hsc70-Interacting Protein

An epitope which was mostly identified in serum samples (4 out of eleven erosive and non-erosive RA samples) and which is also related to stress responses is derived from the Hsc70-interacting protein Hip: Hip (83-98) with the amino acid sequence of SEQ ID NO: 38 (Table 3). Two length variants of this epitope were identified (Table 3): the 18-mer Hip (83-100; SEQ ID NO: 36) and the 15-mer Hip (84-98; SEQ ID NO: 39). An additional length variant was discovered in one erosive synovia sample (Table 3): the 15-mer Hip (85-99; SEQ ID NO: 37). As judged from the shortest length variant Hip (84-98) a DRB1*0401 binding motif attaining a moderate score of 8%, can be postulated: 89I as P1 anchor, 92D as P4 anchor, 94D as P6 anchor.

In the cytosol of eukaryotic cells, Hip and Hop proteins associate with Hsc70 in order to participate in the regulation of Hsc70 chaperone activity (Frydman, J. & Höhfeld, J., Trends Biochem Sci 22 (1997) 87-92). The 42 kD Hip protein binds to the ATPase domain of Hsc70. It was postulated that Hip might increase the half-life of the chaperone-substrate complex providing the molecular basis for an efficient cooperation of Hsc70 with downstream chaperone systems. Hsc70 and Hsp90 have been shown to cooperate during protein folding in vitro (Jakob, U. & Buchner, J., Trends Biochem Sci 19 (1994) 205-211; Freeman, B. C. & Morimoto, R. I., EMBO J. 15 (1996) 2969-2979) and to play a role in thermal denaturation (Schneider, C. et al., PNAS 93 (1996) 14536-14541). The Hsc70 and Hsp90 association with stress-adaptation ultimately links Hip to stress responses, including the induction of heat shock proteins, in the synovial tissue of RA patients (reviewed in: Schett, G. et al., Arthritis Res 3 (2001) 80-86).

Binding Properties of ITIH4, C4, C3, SH3BGRL3, Fig1, HPX and HIP

In order to further investigate the binding properties of the above described antigenic peptides of ITIH4, C4, C3, SH3BGRL3, Fig1, HPX and Hip to HLA-DR4 molecules, in vitro binding assays were performed (FIG. 3): According to their IC₅₀ values against the reporter peptide HA (309-317), the synthetic peptides ITIH4 (274-287), SH3BGRL3 (13-26), and Fig1 (293-309) bind to HLA-DR4 with high affinity (FIG. 3). Moderate binding to HLA-DR4 was measured for the synthetic peptides C4 (1696-1709) and HPX (351-365). The synthetic peptide C3 (1431-1444) bound only weakly to HLA-DR4 which is in agreement with TEPITOPE scoring (9%). No binding was measured for the Hip (84-98) peptide (data not shown).

Invariant Chain (Ii)

Among the candidate marker peptides that appear to be indicative for erosive and non-erosive RA is an epitope which is derived from the HLA-DR-associated invariant chain (Ii): the 15-mer Ii (110-124; SEQ. ID NO: 83) with the amino acid sequence ATPLLMQALPMGALP (Table 3). Moreover, four length variants were identified: the 16-mer Ii (109-124; SEQ. ID NO: 81), the 17-mer Ii (109-125; SEQ. ID NO: 80), the 18-mer Ii (109-126; SEQ. ID NO: 79) and the 21-mer Ii (109-129; SEQ. ID NO: 82) (Table 3). The presence of length variants gives confidence in the identified peptide sequence which displays a moderate DRB1*0401 binding motif (TEPITOPE binding score: 5%) with 114L serving as a P1 anchor, 117A as a P4 anchor and 119P as a P6 anchor. The epitope was identified in two erosive and three non-erosive RA samples. Two Ii length variants were found in one unpulsed sample and two samples of healthy test persons.

Peptide loading of MHC class II molecules is regulated by two accessory molecules in the class II pathway, invariant chain Ii and HLA-DM (reviewed by Bakke, O. & Nordeng T. W., Immunol Rev 172 (1999) 171-187 and Kropshofer, H. et al., Immunol Today 18 (1997) 77-82). Ii trimers bind to nascent MHC class II molecules in the rough endoplasmic reticulum (ER) and block the peptide-binding groove, stabilizing the class II molecule and preventing the binding of ligands available in the ER. Class II/Ii complexes are transported via the Golgi apparatus to endosomes, where Ii is degraded to a nested set of class II-associated Ii peptides (CLIP). The release of CLIP enables endosomal peptides to bind to the MHC class II molecules which are presented to T cell receptors on the cell surface. The identified epitope Ii (110-124) overlaps with the C-terminal part of CLIP. Interestingly, a reduced interaction of CLIP with RA-associated HLA-DR alleles was described (Patil, N. S. et al., J Immunol 167 (2001) 7157-7168), indicating that a reduced interaction may contribute to the pathophysiology of autoimmunity in RA.

Retinoic Acid Receptor Responder Protein 2

In 4 out of 11 serum samples (erosive & non-erosive RA) an epitope was identified which is derived from retinoic add receptor responder protein 2 (RARRES2): the 22-mer RARRES2 (40-61; SEQ. ID NO: 86) with the amino acid sequence HPPVQWAFQETSVESAVDTPFP (Table 3). In the C-terminal part of the epitope two length variants could have been identified: the 23-mer RARRES2 (40-62; SEQ. ID NO: 84) and the 24-mer RARRES2 (40-63; SEQ. ID NO: 85) (Table 3). With 45W as P1 anchor, 48Q as P4 anchor and 50T as P6 anchor, the epitope displays a moderate binding motif in the context of DRB1*0401 (TEPITOPE binding score: 3%).

RARRES2 is a small 18.6 kD protein and mostly expressed in the endothelium and epidermis. The expression appears to be hormone dependent and a response to retinoic acid in skin and some osteotrophic hormones in marrow-derived stromal cells was identified (Nagpal, S. et al., J Invest Dermatol 109 (1997) 91-95 and Adams, A. E. et al., J Cell Biochem 74 (1999) 587-595). The function of RARRES2 is largely unknown and an association with RA, for instance through impaired osteoclastogenesis, is imaginable.

### Fibronectin

Another HLA-DR4 associated peptide which was exclusively found in 3 out of 4 synovia samples (erosive & non-erosive RA) is derived from fibronectin (Fn), a major glycoprotein in blood plasma and in the extracellular matrix: the 15-mer Fn (1881-1895; SEQ. ID NO: 90) with the amino acid sequence IYLYTLNDNARSSPV (Table 3). The epitope appears to be a very good DRB1*0401 binder with 1885Y serving as a hydrophobic P1 anchor, 1888N as a P4 anchor and 1890N as a P6 anchor (TEPITOPE binding score: 1%). Four length variants were additionally identified: the 16-mer Fn (1881-1896; SEQ. ID NO: 91), the 16-mer Fn (1880-1895; SEQ. ID NO: 88), the 17-mer Fn (1881-1897; SEQ. ID NO: 89) and the 17-mer Fn (1880-1896; SEQ. ID NO: 87), strongly supporting the validity of the identified Fn epitope (Table 3).

Fibronectin plays an important role in cell adhesion, cell motility and in opsonization. It binds collagen and fibrin and mediates adhesion of fibroblasts to collagen fibrils. Fibronectin is strongly expressed in the synovial lining layer of both RA and osteoarthritis patients and correlates with hyperplasia of diseased joints. The identification of epitope Fn (1881-1895) in only samples of synovial origin is in line with these findings and points to a putative role of highly abundant fibronectin in autoimmunity.

Cathepsin B

Three out of fifteen RA samples (erosive & non-erosive RA) gave rise to an epitope which originates from Cathepsin B (CatB): the 15-mer CatB (227-241; SEQ. ID NO: 92) with the amino acid sequence YNSYSVSNSEKDIMA (Table 3). The peptide displays a moderate DRB1*0401 binding motif with 230Y as a hydrophobic P1 anchor and the serine residues at positions 233 and 235 as putative P4 and P6 anchors (TEPITOPE binding score: 6%).

Cathepsin B is a thiol peptidase and believed to participate in intracellular degradation and turnover of proteins, e.g. collagen. It is located in lysosomes of different cell types including leukocytes. In the context of inflammation and disease it was shown that Cathepsin B contributes to cartilage destruction in osteoarthritis and pathological proteolysis in RA and cancer (Cunnane, G. et al., Arthritis Rheum 44 (2001) 1744-1753). Enzyme activities of Cathepsin B and other lysosomal peptidases correlate with RA progression which supports the validity of epitope CatB (227-241) as a putative RA marker peptide (Sohar, N. et al., Biol Chem 383 (2002) 865-869).

Tripeptidyl-Peptidase II

Another peptidase-derived epitope which was found in three out of fifteen RA samples (erosive & non-erosive RA) is derived from tripeptidyl-peptidase II (TPP2): the 15-mer TPP2 (970-984; SEQ. ID NO: 93) having the amino acid sequence AGSLTLSKTELGKKA (Table 3). Additionally, the length variant TPP2 (970-985; SEQ. ID NO: 94) was identified. The peptidase epitope contains a typical DRB1*0401 binding motif with 973L, 976S and 978T as P1, P4 and P6 anchors respectively (TEPITOPE binding score: 3%).

Similar to Cathepsin B, tripeptidyl-peptidase II is involved in lysosomal protein degradation. The identification of epitope TPP2 (970-984) in the context of RA provides the first indication that TPP2 might be implicated in impaired protein degradation in the context of inflammation and RA.

Legumain

Legumain (LGMN) completes the set of three peptidases which were found in this investigation. The identified epitope LGMN (99-112; SEQ. ID NO: 95) with the amino acid sequence VPKDYTGEDVTPQN (Table 3) displays a typical binding motif with respect to the HLA allele DRB1*0401 in which 103Y could serve as a P1 anchor, 106E as a P4 anchor and 108V as a P6 anchor (TEPITOPE binding score: 1%).

Legumain occurs in endosomal and lysosomal fractions of antigen-presenting cells such as dendritic cells (Schwarz, G. et al., Biol Chem 383 (2002) 1813-1816). It has a strict specificity for the hydrolysis of asparaginyl bonds and was shown to play an important role in the processing of bacterial antigens for MHC class II presentation (Manoury, B. et al., Nature 396 (1998) 695-699). Whether Legumain is involved in autoimmune diseases, such as RA, remains unknown but the fact that three lysosomal peptidases together with GILT and the 26S proteasome were identified in this investigation suggests that the protein degradation machinery facilitating antigen processing might be significantly altered in the context of RA.

Platelet Activating Factor Receptor

An epitope which was identified in three out of eleven serum RA samples (erosive & non-erosive RA) is derived from a receptor for platelet activating factor (PAFR): the 13-mer PAFR (264-276; SEQ. ID NO: 96) with the amino acid sequence DSKFHQAINDAHQ (Table 3). According to TEPITOPE scoring (3%) the epitope contains a moderated DRB1*0401 binding motif with 267F as P1 anchor, 270A as P4 anchor and 272N as P6 anchor (Table 3).

Platelet activating factor (PAF) is a pro-inflammatory lipid mediator which binds to a G-protein-coupled seven transmembrane receptor on the surface of a broad range of cell types. By receptor binding, PAF transduces pleiotrophic functions which include cell motility, smooth muscle contraction, and synthesis and release of cytokines (reviewed by Honda, Z. et al., J Biochem 131 (2002) 773-779). Pharmacological studies and the establishment of PAFR (−/−) mice have suggested that PAF functions in a variety of settings including allergy, inflammation, neural functions, reproduction, and atherosclerosis. Interestingly, PAF was found in elevated levels in the synovial fluid of RA patients and shown to induce neoangiogenesis, which is frequently observed in rheumatoid synovitis (Lupia, E. et al., Eur J Immunol 26 (1996) 1690-1694). This type of evidence gives further confidence in epitope PAFR (264-276) being a RA-associated marker peptide.

Poly-alpha-2,8-sialyltransferase

Another epitope which was found in three out of eleven serum samples (erosive & non-erosive RA) originates from Polysialyltransferase (PST): the 15-mer PST (333-347; SEQ. ID NO: 97), MPLEFKTLNVLHNRG (Table 3), displays a moderate binding motif with respect to DRB1*0401 binding (TEPITOPE scoring: 2%). 337F could serve as a P1 anchor, 340L as a P4 anchor and 342V as a P6 anchor.

Polysialic acid is a carbohydrate composed of a linear homopolymer of α-2,8-linked sialic acid residues. The glycan is mainly attached to the neural cell adhesion molecule (N-CAM) and implicated in many morphogenic processes of the neural cells by modulating the adhesive property of N-CAM. The membrane protein polysialyltransferase catalyzes the polycondensation of sialic acid residues and is highly expressed in fetal brain, lung and kidney, in adult heart, spleen and thymus, and to a lesser extent in peripheral blood leukocytes (Nakayama, J. et al., Proc Natl Acad Sci 92 (1995) 7031-7035). Elevated PST levels were observed in serum of patients with metastatic tumors and an increased enzyme activity was also associated with rheumatoid arthritis (Berge, P. G. et al., Klin Wochenschr 60 (1982) 445-449). The identification of epitope PST (333-347) in several RA samples is in line with these observations and supports the putative role of PST in RA.

Ras-Related Protein Rab-11B

The last epitope presented in this analysis of RA samples is derived from the Ras-related protein Rab-11B: the 13-mer Rab-11B (51-63; SEQ. ID NO: 102) with the amino acid sequence RSIQVDGKTIKAQ (Table 3, the peptide sequence were validated with regard to binding to the RA susceptibility allele DRB1*0301 by using the TEPITOPE software (Hammer, J. et al., Adv Immunol 66 (1997) 67-100)). This epitope and four additional length variants, the 14-mer Rab-11B (50-63; SEQ. ID NO: 100), the 15-mer Rab-11B (49-63; SEQ. ID NO: 98), the 17-mer Rab-11B (49-65; SEQ. ID NO: 101) and the 18-mer Rab-11B (49-66; SEQ. ID NO: 99) were found in three erosive and two non-erosive RA samples (Table 3). The 15-mer Rab-11B (49-63) was identified in 1 out of 12 control samples.

.Rab proteins are small GTPases that play an important role in membrane trafficking along the endo- and exocytic pathway. Rab-11B is assumed to be essential for the transport of internalized transferrin from the recycling compartment to the plasma membrane. Furthermore Rab-11B was identified in rat osteoclasts and might play an additional role in bone resorption. The epitope Rab-11B (51-63) provides the first indication of a role of Rab-11B in RA development.

TABLE 1
HLA-DR associated peptide antigens from serum and synovial fluid of patients with mostly non-erosive RA.

| SEQ. ID. | RA- | RF^{b} | | Haplo- | | | DRB1*0401- | |
|---|---|---|---|---|---|---|---|---|
| NO. | type^{a} | (IU/ml) | Sample^{c} | type^{d} | Length | Sequence^{e} | binding score^{f} | Protein source^{g} |
| 1 | N | − | S | 1 | 14 | 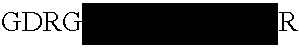 | 1% | Interferon-gamma-inducible |
| 2 | N | 6.8 | S | 3 | 17 | 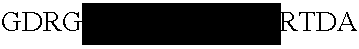 | | lysosomal thiol reductase |
| 3 | N | 6.8 | S | 3 | 16 | 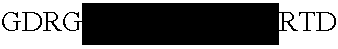 | | (192-205) |
| 2 | N | 9.1 | Syn | 4 | 17 | 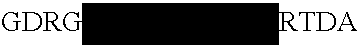 | | |
| 3 | N | 9.1 | Syn | 4 | 16 | 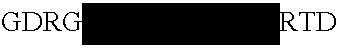 | | |
| 3 | E | 20.7 | S | 3 | 16 | 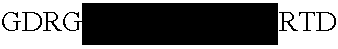 | | |
| 58 | N | 9.1 | Syn | 4 | 17 | 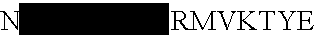 | 2% | Integrin beta-2 |
| 58 | N | 9.1 | S | 4 | 17 | 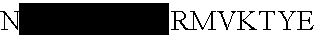 | | (315-331) |
| 59 | N | 9.1 | S | 4 | 19 | 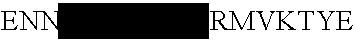 | | |
| 60 | N | 153 | S | 5 | 17 | 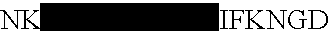 | 3% | Phosphatidylinositol-4,5- |
| 60 | N | 88 | S | 5 | 17 | 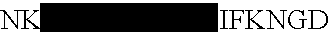 | | bisphosphate 3-kinase |
| | | | | | | | | (792-808) |
| 61 | N | 9.1 | S | 4 | 16 | 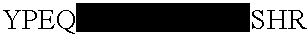 | 2% | Urokinase-type |
| 61 | N | 9.1 | Syn | 4 | 16 | 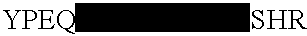 | | plasminogen activator |
| | | | | | | | | (328-343) |
| 62 | N | 153 | S | 5 | 16 | 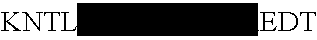 | 1% | Immunglobulin heavy chain |
| 62 | N | 88 | S | 5 | 16 | 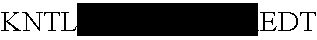 | | V-III region (V_{H}26) |
| | | | | | | | | (95-110) |
| 63 | N | 153 | S | 5 | 16 | 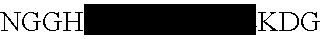 | 8% | DJ-1 protein |
| 63 | N | 88 | S | 5 | 16 | 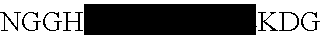 | | (135-150) |

| | | | |
|---|---|---|---|
| strong HLA-DRB1*0401 binder | 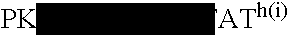 | 1% | Influenza Haemagglutinin |
| | | | (307-319) |
| moderate HLA-DRB1*0401 binder | 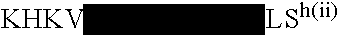 | 2% | Immunglobuline kappa |
| | | | (188-202) |
| weak HLA-DRB1*0401 binder | 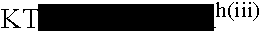 | >10% | M. tuberculosis Hsp65 |
| | | | (3-13) |

^{a}RA-type of the patient based on clinical diagnosis: persistant erosive (E) or persistent non-erosive (N) RA
^{b}Rheumatoid factor
^{c}Sample description: dendritic cells pulsed with serum (S) or synovial fluid (Syn)
^{d}Haplotype of the buffy coat: (1) HLA-DRB1*0401, *03011; (2) HLA-DRB1*0401, *0304; (3) HLA-DRB1*0401, *1301; (4) HLA-DRB1*0401, *0701; (5) HLA-DRB1*0401, *0407 *0401, *1301; (4) HLA-DRB1*0401, *0701
^{e}Sequences of the RA-derived peptides in one-letter-code. The HLA-DRB1*0401 binding motif is boxed in grey.
^{f}Score of the epitope in context of the HLA-DRB1*0401 allele based on the TEPITOPE program (Hammer, J. et al., Adv Immunol 66 (1997) 67-100).
^{g}Protein name according to the Swiss-Prot/TreEMBL database. The numbers in brackets represent the shortest length variant of the repective epitope.
^{h(i)}Rothbard, J. B. et al., Cell 52 (1988) 515-523.
^{h(ii)}Chicz, R. M. et al., J Exp Med 178 (1993) 27-47.
^{h(iii)}van Schooten, W. C. et al., Eur J Immunol 19 (1989) 2075-2079.

TABLE 2
HLA-DR associated peptide antigens from serum and synovial fluid of patients with mostly erosive RA.

| SEQ. ID. | RA- | RF^{b} | | Haplo- | | | DRB1*0401- | |
|---|---|---|---|---|---|---|---|---|
| NO. | type^{a} | (IU/ml) | Sample^{c} | type^{d} | Length | Sequence^{e} | binding score^{f} | Protein source^{g} |
| 4 | E | − | S | 1 | 16 | 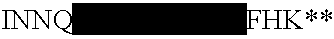 | 3% | Apolipoprotein B-100 |
| 4 | E | + | S | 2 | 16 | 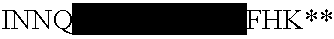 | | (2877-2892) |
| 4 | E | 134 | S | 3 | 16 | 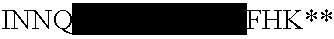 | | |
| 4 | E | 20.7 | S | 3 | 16 | 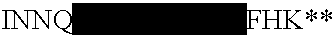 | | |
| 5 | E | 20.7 | S | 3 | 17 | 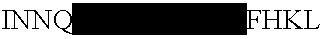 | | |
| 4 | N | − | S | 1 | 16 | 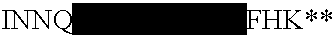 | | |
| 64 | E | 134 | S | 3 | 15 | 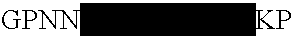 | 3% | 26S proteasome non-ATPase |
| 64 | E | 0 | S | 5 | 15 | 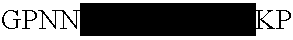 | | regulatory subunit 8 |
| 64 | E | 401 | S | 5 | 15 | 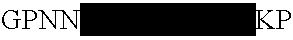 | | (218-232) |
| 65 | E | 134 | Syn | 3 | 16 | 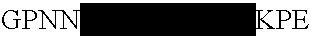 | | |
| 66 | E | 134 | Syn | 3 | 17 | 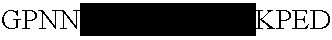 | | |
| 66 | N | 6.8 | Syn | 3 | 17 | 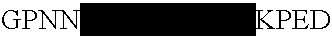 | | |
| 67 | N | 6.8 | Syn | 3 | 18 | 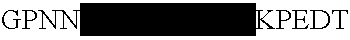 | | |
| 68 | E | 0 | S | 5 | 16 | 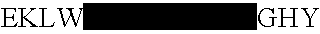 | 1% | Interleukin-1 receptor |
| 68 | E | 401 | S | 5 | 16 | 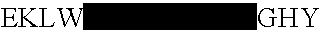 | | (79-94) |
| 69 | E | − | S | 1 | 14 | 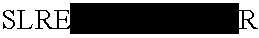 | 1% | Fibromodulin |
| 70 | E | 20.7 | Syn | 3 | 13 | 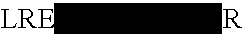 | | (178-190) |
| 69 | E | 20.7 | Syn | 3 | 14 | 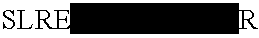 | | |
| 70 | E | 134 | Syn | 3 | 13 | 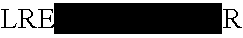 | | |
| 69 | N | 6.8 | Syn | 3 | 14 | 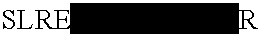 | | |
| 70 | N | 6.8 | Syn | 3 | 13 | 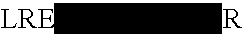 | | |
| 71 | E | 134 | S | 3 | 15 | 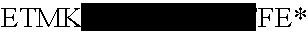 | 3% | GM-CSF/IL-3/IL-5 receptor |
| 71 | E | 20.7 | S | 3 | 15 | 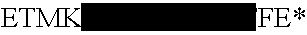 | | (359-373) |
| 71 | E | 0 | S | 5 | 15 | 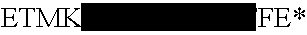 | | |
| 72 | E | 401 | S | 5 | 17 | 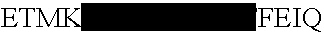 | | |
| 72 | E | 134 | Syn | 3 | 17 | 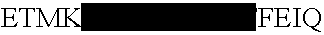 | | |
| 71 | N | 6.8 | S | 3 | 15 | 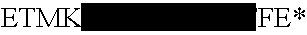 | | |
| 71 | N | 6.8 | Syn | 3 | 15 | 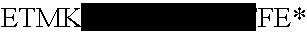 | | |
| 73 | E | 0 | S | 5 | 16 | 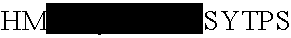 | 2% | Sorting nexin 3 |
| 73 | E | 401 | S | 5 | 16 | 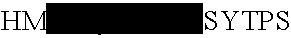 | | (142-157) |

^{a}RA-type of the patient based on clinical diagnosis: persistant erosive (E) or persistent non-erosive (N) RA
^{b}Rheumatoid factor
^{c}Sample description: dendritic cells pulsed with serum (S) or synovial fluid (Syn)
^{d}Haplotype of the buffy coat: (1) HLA-DRB1*0401, *03011; (2) HLA-DRB1*0401, *0304; (3) HLA-DRB1*0401, *1301; (4) HLA-DRB1*0401, *0701; (5) HLA-DRB1*0401, *0407
^{e}Sequences of the RA-derived peptides in one-letter-code. The HLA-DRB1*0401 binding motif is boxed in grey.
^{f}Score of the epitope in context of the HLA-DRB1*0401 allele based on the TEPITOPE program (Hammer, J. et al., Adv Immunol 66 (1997) 67-100).
^{g}Protein name according to the Swiss-Prot/TrEMBL database. The numbers in brackets represent the shortest length variant of the repective epitope.
*Length variant of the respective epitope which was indentified in 1 healthy control sample as well.
** Length variant of the respective epitope, which was indentified in 2 healthy control samples as well.

TABLE 3
HLA-DR associated peptide antigens from serum or synvial fluid of patients with erosive and non-erosive RA.

| SEQ. ID. | RA- | RF^{b} | | Haplo- | | | DRB1*0401- | |
|---|---|---|---|---|---|---|---|---|
| NO. | type^{a} | (IU/ml) | Sample^{c} | type^{d} | Length | Sequence^{e} | binding score^{f} | Protein source^{g} |
| 6 | E | − | S | 3 | 19 | 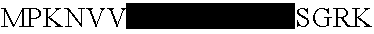 | 1% | Inter-alpha-trypsin inhibitor |
| 7 | E | + | S | 3 | 18 | 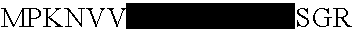 | | heavy chain H4 |
| 8 | E | + | S | 3 | 17 | 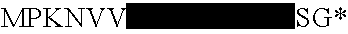 | | (274-287) |
| 8 | E | 134 | S | 3 | 17 | 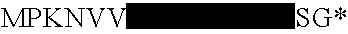 | | |
| 9 | E | 134 | S | 3 | 14 | 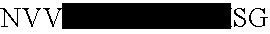 | | |
| 8 | E | 134 | S | 2 | 17 | 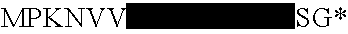 | | |
| 10 | E | 134 | S | 2 | 15 | 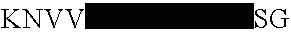 | | |
| 8 | E | 20.7 | S | 1 | 17 | 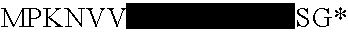 | | |
| 11 | E | 20.7 | S | 1 | 15 | 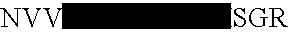 | | |
| 6 | N | − | S | 3 | 19 | 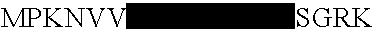 | | |
| 7 | N | 6.8 | S | 3 | 18 | 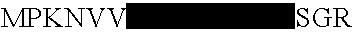 | | |
| 8 | N | 6.8 | S | 3 | 17 | 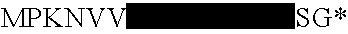 | | |
| 8 | N | 6.8 | S | 3 | 17 | 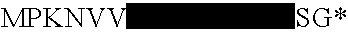 | | |
| 12 | N | 6.8 | S | 3 | 16 | 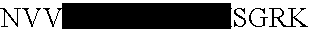 | | |
| 8 | N | 6.8 | S | 1 | 17 | 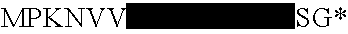 | | |
| 8 | N | 153 | S | 5 | 17 | 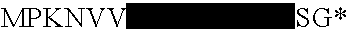 | | |
| 8 | N | 88 | S | 5 | 17 | 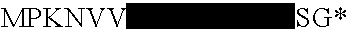 | | |
| 10 | E | 0 | S | 5 | 15 | 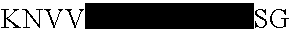 | | |
| 8 | E | 0 | S | 5 | 17 | 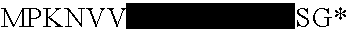 | | |
| 7 | E | 0 | S | 5 | 18 | 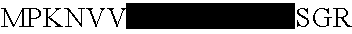 | | |
| 8 | E | 401 | S | 5 | 17 | 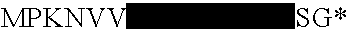 | | |
| 7 | E | 401 | S | 5 | 18 | 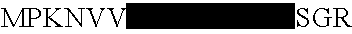 | | |
| 13 | N | − | S | 1 | 15 | 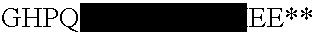 | 1% | Complement C4 |
| 14 | N | − | S | 1 | 16 | 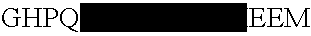 | | (1697-1708) |
| 15 | N | − | S | 1 | 14 | 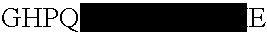 | | |
| 13 | N | 6.8 | S | 3 | 15 | 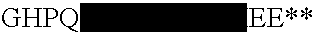 | | |
| 16 | N | 9.1 | S | 4 | 18 | 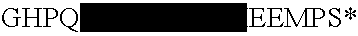 | | |
| 17 | N | 9.1 | S | 4 | 13 | 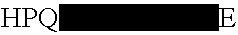 | | |
| 13 | N | 9.1 | S | 4 | 15 | 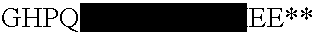 | | |
| 18 | N | 9.1 | S | 4 | 12 | 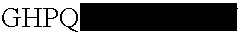 | | |
| 14 | E | − | S | 1 | 16 | 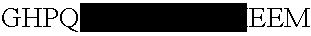 | | |
| 13 | E | − | S | 1 | 15 | 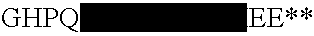 | | |
| 18 | E | − | S | 1 | 12 | 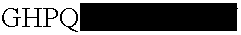 | | |
| 13 | E | + | S | 2 | 15 | 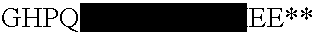 | | |
| 13 | E | 134 | S | 3 | 15 | 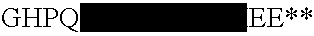 | | |
| 15 | E | 134 | S | 3 | 14 | 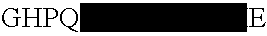 | | |
| 16 | E | 134 | S | 3 | 18 | 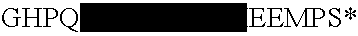 | | |
| 13 | E | 20.7 | S | 3 | 15 | 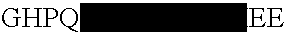 | | |
| 16 | E | 20.7 | S | 3 | 18 | 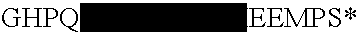 | | |
| 13 | E | 0 | S | 5 | 15 | 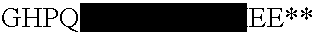 | | |
| 19 | N | 9.1 | S | 4 | 19 | 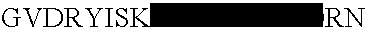 | 9% | Complement C3 (α-chain) |
| 20 | N | − | S | 1 | 15 | 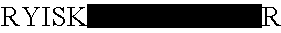 | | (1431-1443) |
| 21 | N | − | S | 1 | 14 | 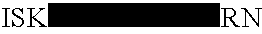 | | |
| 22 | N | − | S | 1 | 15 | 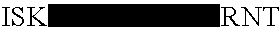 | | |
| 23 | N | − | S | 1 | 13 | 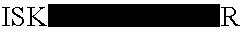 | | |
| 21 | E | + | S | 2 | 14 | 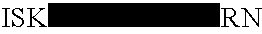 | | |
| 74 | N | 88 | S | 5 | 17 | 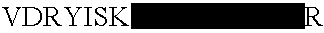 | | |
| 75 | E | 0 | S | 5 | 14 | 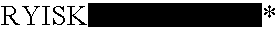 | | |
| 76 | N | 153 | S | 5 | 19 | 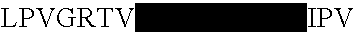 | 3% | Complement C3 (β-chain) |
| 76 | N | 88 | S | 5 | 19 | 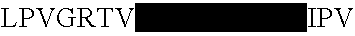 | | (157-175) |
| 76 | E | 0 | S | 5 | 19 | 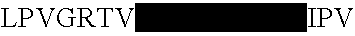 | | |
| 77 | E | 0 | S | 5 | 20 | 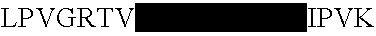 | | |
| 76 | E | 401 | S | 5 | 19 | 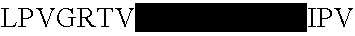 | | |
| 77 | E | 401 | S | 5 | 20 | 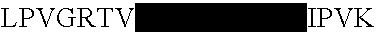 | | |
| 24 | N | 6.8 | S | 3 | 16 | 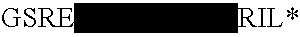 | 4% | SH3 domain-binding |
| 25 | N | − | S | 1 | 12 | 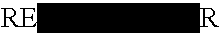 | | glutamic acid-rich-like |
| 26 | N | − | S | 1 | 14 | 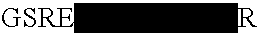 | | protein 3 |
| 24 | N | − | S | 1 | 16 | 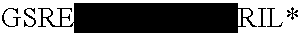 | | (15-26) |
| 27 | N | − | S | 1 | 14 | 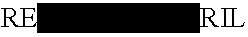 | | |
| 26 | E | − | S | 1 | 14 | 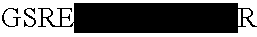 | | |
| 25 | E | − | S | 1 | 12 | 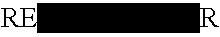 | | |
| 25 | E | 134 | S | 3 | 12 | 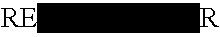 | | |
| 25 | E | 20.7 | S | 3 | 12 | 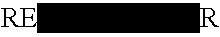 | | |
| 24 | E | 20.7 | S | 3 | 16 | 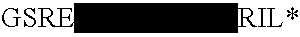 | | |
| 26 | E | 401 | S | 5 | 14 | 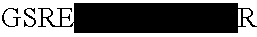 | | |
| 25 | E | 401 | S | 5 | 12 | 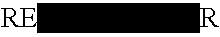 | | |
| 28 | E | 20.7 | Syn | 3 | 17 | 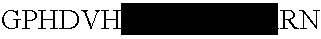 | 1% | Interleukin-4- |
| 29 | E | 20.7 | Syn | 3 | 19 | 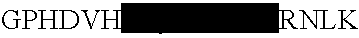 | | induced protein 1 |
| 29 | E | 134 | Syn | 3 | 19 | 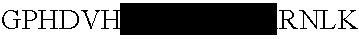 | | (293-308) |
| 28 | E | − | S | 1 | 17 | 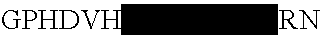 | | |
| 28 | E | + | S | 2 | 17 | 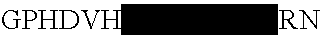 | | |
| 28 | E | 134 | S | 3 | 17 | 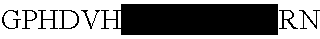 | | |
| 30 | E | 134 | S | 3 | 16 | 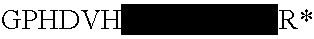 | | |
| 30 | E | 20.7 | S | 3 | 16 | 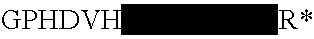 | | |
| 28 | N | 6.8 | S | 3 | 17 | 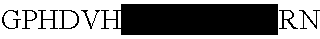 | | |
| 28 | N | 9.1 | S | 4 | 17 | 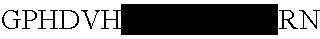 | | |
| 30 | N | 9.1 | S | 4 | 16 | 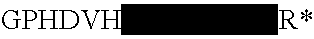 | | |
| 28 | N | − | S | 1 | 17 | 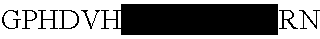 | | |
| 28 | N | 9.1 | Syn | 4 | 17 | 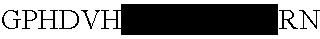 | | |
| 28 | N | 6.8 | Syn | 3 | 17 | 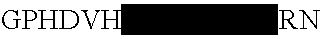 | 1% | |
| 28 | E | 0 | S | 5 | 17 | 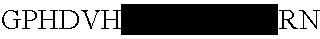 | | Interleukin-4-induced protein 1 |
| | | | | | | | | (293-309) |
| 31 | N | − | S | 1 | 18 | 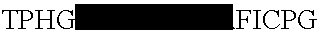 | 1% | Hemopexin |
| 32 | N | 9.1 | S | 4 | 17 | 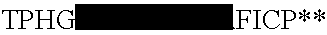 | | (351-363) |
| 33 | N | 9.1 | S | 4 | 13 | 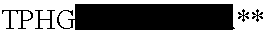 | | |
| 32 | N | 6.8 | Syn | 3 | 17 | 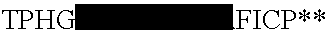 | | |
| 32 | N | 9.1 | Syn | 4 | 17 | 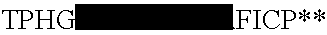 | | |
| 33 | N | 9.1 | Syn | 4 | 13 | 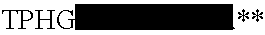 | | |
| 34 | N | 9.1 | Syn | 4 | 14 | 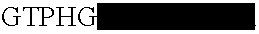 | | |
| 35 | N | 6.8 | S | 3 | 15 | 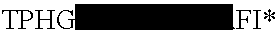 | | |
| 32 | N | 6.8 | S | 3 | 17 | 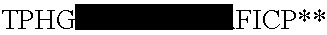 | | |
| 31 | E | + | S | 2 | 18 | 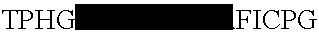 | | |
| 35 | E | 134 | S | 3 | 15 | 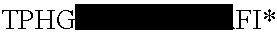 | | |
| 32 | E | 134 | S | 3 | 17 | 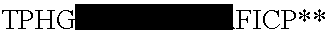 | | |
| 35 | E | 20.7 | S | 3 | 15 | 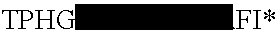 | | |
| 32 | E | 20.7 | S | 3 | 17 | 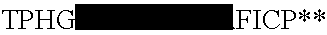 | | |
| 78 | N | 153 | S | 5 | 18 | 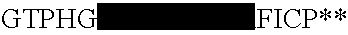 | | |
| 35 | N | 88 | S | 5 | 15 | 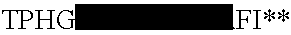 | | |
| 33 | N | 88 | S | 5 | 13 | 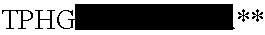 | | |
| 32 | N | 88 | S | 5 | 17 | 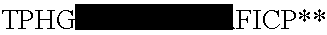 | | |
| 78 | E | 0 | S | 5 | 18 | 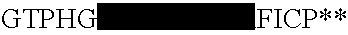 | | |
| 35 | E | 0 | S | 5 | 15 | 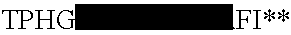 | | |
| 33 | E | 401 | S | 5 | 13 | 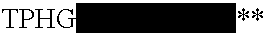 | | |
| 35 | E | 401 | S | 5 | 15 | 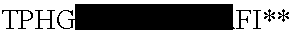 | | |
| 32 | E | 401 | S | 5 | 17 | 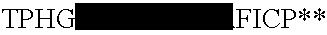 | | |
| 36 | E | 134 | Syn | 3 | 18 | 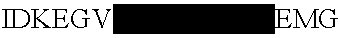 | 8% | Hsc70-interacting protein |
| 37 | E | 134 | Syn | 3 | 15 | 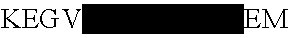 | | (84-98) |
| 38 | E | − | S | 1 | 16 | 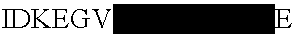 | | |
| 39 | E | − | S | 1 | 15 | 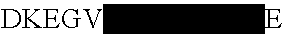 | | |
| 36 | N | 9.1 | S | 4 | 18 | 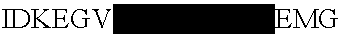 | | |
| 38 | N | − | S | 1 | 16 | 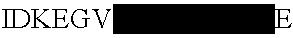 | | |
| 38 | N | 6.8 | S | 3 | 16 | 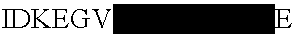 | | |
| 79 | E | + | S | 2 | 18 | 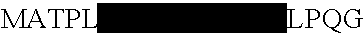 | 5% | Invariant chain (Ii) |
| 80 | E | + | S | 2 | 17 | 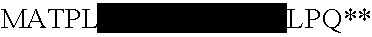 | | (110-124) |
| 81 | E | + | S | 2 | 16 | 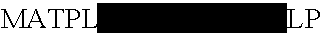 | | |
| 80 | E | 134 | Syn | 3 | 17 | 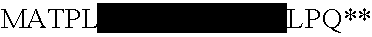 | | |
| 82 | N | 9.1 | S | 4 | 21 | 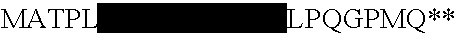 | | |
| 83 | N | 6.8 | S | 3 | 15 | 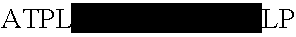 | | |
| 82 | N | 6.8 | Syn | 3 | 21 | 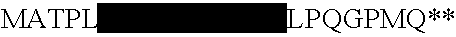 | | |
| 84 | E | 0 | S | 5 | 23 | 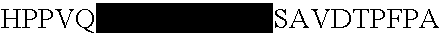 | 3% | Retinoic acid receptor |
| 85 | E | 0 | S | 5 | 24 | 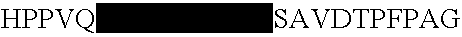 | | responder protein 2 |
| 86 | E | 0 | S | 5 | 22 | 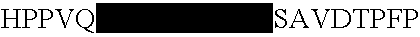 | | (40-61) |
| 84 | E | 401 | S | 5 | 23 | 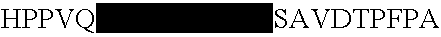 | | |
| 85 | E | 401 | S | 5 | 24 | 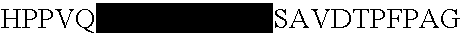 | | |
| 84 | N | 153 | S | 5 | 23 | 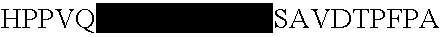 | | |
| 86 | N | 153 | S | 5 | 22 | 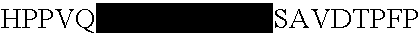 | | |
| 85 | N | 153 | S | 5 | 24 | 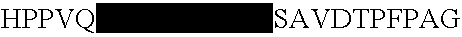 | | |
| 84 | N | 88 | S | 5 | 23 | 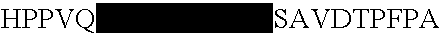 | | |
| 85 | N | 88 | S | 5 | 24 | 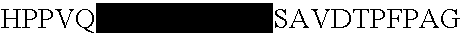 | | |
| 87 | E | 20.7 | Syn | 3 | 17 | 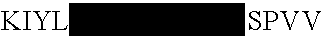 | 1% | Fibronectin |
| 88 | E | 20.7 | Syn | 3 | 16 | 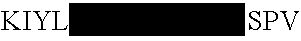 | | (1881-1895) |
| 89 | E | 20.7 | Syn | 3 | 17 | 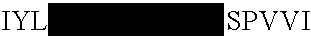 | | |
| 90 | E | 20.7 | Syn | 3 | 15 | 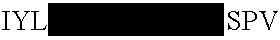 | | |
| 91 | E | 20.7 | Syn | 3 | 16 | 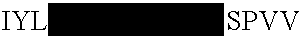 | | |
| 90 | E | 134 | Syn | 3 | 15 | 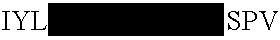 | | |
| 90 | N | 6.8 | Syn | 3 | 15 | 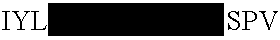 | | |
| 92 | E | 134 | S | 3 | 15 | 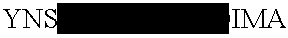 | 6% | Cathespin B |
| 92 | E | 134 | Syn | 3 | 15 | 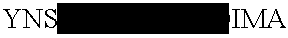 | | (227-241) |
| 92 | N | 6.8 | S | 3 | 15 | 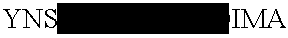 | | |
| 93 | E | 0 | S | 5 | 15 | 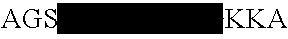 | 3% | Tripeptidyl-peptidase II |
| 93 | E | 401 | S | 5 | 15 | 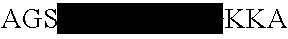 | | (970-984) |
| 94 | E | 401 | S | 5 | 16 | 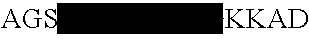 | | |
| 94 | N | 153 | S | 5 | 16 | 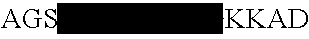 | | |
| 95 | E | − | S | 1 | 14 | 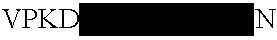 | 1% | Legumain |
| 95 | N | − | S | 1 | 14 | 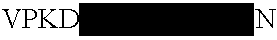 | | (99-112) |
| 95 | N | 9.1 | Syn | 4 | 14 | 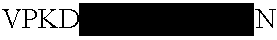 | | |
| 96 | E | 0 | S | 5 | 13 | 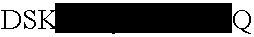 | 3% | Platelet activating factor |
| 96 | E | 401 | S | 5 | 13 | 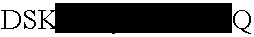 | | receptor |
| 96 | N | 88 | S | 5 | 13 | 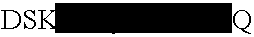 | | (264-276) |
| 97 | E | 0 | S | 5 | 15 | 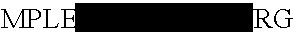 | 2% | Poly-alpha-2,8-sialyltransferase |
| 97 | E | 401 | S | 5 | 15 | 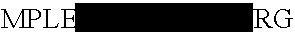 | | (333-347) |
| 97 | N | 88 | S | 5 | 15 | 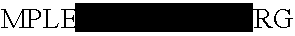 | | |
| 98 | E | − | S | 1 | 15 | 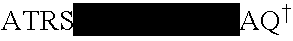 | 1%^{†} | Ras-related protein Rab-11B |
| 98 | E | + | S | 2 | 15 | 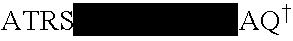 | | (51-63) |
| 98 | E | 20.7 | S | 3 | 15 | 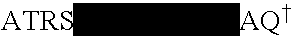 | | |
| 99 | E | 20.7 | S | 3 | 18 | 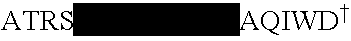 | | |
| 100 | N | 6.8 | S | 3 | 14 | 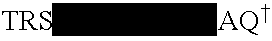 | | |
| 99 | N | 6.8 | S | 3 | 18 | 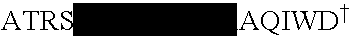 | | |
| 101 | N | 6.8 | S | 3 | 17 | 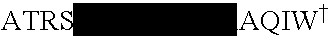 | | |
| 98 | N | − | S | 1 | 15 | 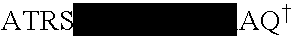 | | |
| 102 | N | − | S | 1 | 13 | 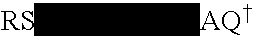 | | |

^{a}RA-type of the patient based on clinical diagnosis: persistant erosive (E) or persistent non-erosive (N) RA
^{b}Rheumatoid factor
^{c}Sample description: dendritic cells pulsed with serum (S) or synovial fluid (Syn)
^{d}Haplotype of the buffy coat: (1) HLA-DRB1*0401, *03011; (2) HLA-DRB1*0401, *0304; (3) HLA-DRB1*0401, *1301; (4) HLA-DRB1*0401, *0701; (5) HLA-DRB1*0401, *0407
^{e}Sequences of the RA-derived peptides in one-letter-code. The putative HLA-DRB1*0401 (^{†}*0301) binding motif is boxed in grey.
^{f}Score of the epitope in context of the HLA-DRB1*0401 (^{†}*0301) allele based on the TEPITOPE program (Hammer, J. et al., Adv Immunol 66 (1997) 67-100).
^{g}Protein name according to the Swiss-Prot/TrEMBL database. The numbers in brackets represent the shortest length variant of the repective epitope.
*(**)Length variant of the respective epitope which was identified in 1 (2) healthy control sample(s) as well.

TABLE 4
Summary of the candidate RA markers.

| | | Frequency in the | |
|---|---|---|---|
| RA-type | Protein source^{a} | RA samples^{b} | Accession number^{c} |
| mostly non-erosive | Interferon-γ-inducible lysosomal thiol reductase | 3 of 7 N | P13284 |
| | Integrin beta-2 | 2 of 7 N | P05107 |
| | Phosphatidylinositol-4,5-bisphosphate 3-kinase | 2 of 7 N | P42338 |
| | Urokinase-type plasminogen activator | 2 of 7 N | P00749 |
| | Immunglobulin heavy chain V-III region (V_{H}26) | 2 of 7 N | P01764 |
| | DJ-1 protein | 2 of 7 N | Q99497 |
| mostly erosive | Apolipoprotein B-100 | 4 of 8 E | P04114 |
| | 26S proteasome non-ATPase regulatory subunit 8 | 4 of 8 E | P48556 |
| | Interleukin-1 receptor | 2 of 8 E | P14778 |
| | Fibromodulin | 3 of 8 E | Q06828 |
| | GM-CSF/IL-3/IL-5 receptor | 5 of 8 E | P32927 |
| | Sorting nexin 3 | 2 of 8 E | O60493 |
| erosive and non-erosive | Inter-α-trypsin inhibitor heavy chain H4 | 6 of 8 E/4 of 7 N | Q14624 |
| | Complement C4 | 5 of 8 E/3 of 7 N | P01028 |
| | Complement C3 | 2 of 8 E/3 of 7 N | P01024 |
| | SH3 domain-binding glutamic acid-rich-like protein 3 | 4 of 8 E/2 of 7 N | Q9H299 |
| | Interleukin-4-induced protein 1 | 7 of 8 E/5 of 7 N | Q96RQ9 |
| | Hemopexin | 5 of 8 E/7 of 7 N | P02790 |
| | Hsc70-interacting protein | 2 of 8 E/3 of 7 N | P50502 |
| | Invariant chain (Ii) | 2 of 8 E/3 of 7 N | P04233 |
| | Retinoic acid receptor responder protein 2 | 2 of 8 E/2 of 7 N | Q99969 |
| | Fibronectin | 2 of 8 E/1 of 7 N | P02751 |
| | Cathepsin B | 2 of 8 E/1 of 7 N | P07858 |
| | Tripeptidyl-peptidase II | 2 of 8 E/1 of 7 N | P29144 |
| | Legumain | 1 of 8 E/2 of 7 N | Q99538 |
| | Platelet activating factor receptor | 2 of 8 E/1 of 7 N | P25105 |
| | Alpha-2,8-sialyltransferase | 2 of 8 E/1 of 7 N | Q92187 |
| | Ras-related protein Rab-11B | 3 of 8 E/2 of 7 N | Q15907 |

^{a}Protein name according to the Swiss-Prot/TrEMBL database.
^{b}Frequency of the identified epitope in the RA samples. The RA-type of the patient was based on clinical diagnosis: persistant erosive (E) or persistent non-erosive (N) RA.
^{c}relates to the Swiss-Prot databas

1. A MHC class II antigenic peptides comprising
(a) the amino acid sequence of the peptide binding motif selected from the group consisting of SEQ ID NOs. 49 to 57 and SEQ ID NOs. 103 to 122, or
(b) the amino acid sequence of the peptide binding motif selected from the group consisting of SEQ ID NOs. 49 to 57 and SEQ ID NOs. 103 to 122, with additional N- and C-terminal flanking sequences of a corresponding sequence selected from the group consisting of SEQ ID NOs. 1 to 39 and SEQ ID NOs. 58 to 102.

2. A MHC class II antigenic peptide comprising
(a) the amino acid sequence of the peptide binding motif of SEQ ID NO. 49, or
(b) the amino acid sequence of the peptide binding motif of SEQ ID NO. 49 with additional N- and C-terminal flanking sequences of a corresponding sequence selected from the group consisting of SEQ ID NOs. 1 to 3.

3. A MHC class II antigenic peptide comprising
(a) the amino acid sequence of the peptide binding motif of SEQ ID NO. 103, or
(b) the amino acid sequence of the peptide binding motif of SEQ ID NO. 103 with additional N- and C-terminal flanking sequences of the corresponding sequence of SEQ ID NOs. 58 and 59.

4. A MHC class II antigenic peptide comprising
(a) the amino acid sequence of the peptide binding motif of SEQ ID NO. 104, or
(b) the amino acid sequence of the peptide binding motif of SEQ ID NO. 104 with additional N- and C-terminal flanking sequences of the corresponding sequence of SEQ ID NO. 60.

5. A MHC class II antigenic peptide comprising
(a) the amino acid sequence of the peptide binding motif of SEQ ID NO. 105, or
(b) the amino acid sequence of the peptide binding motif of SEQ ID NO. 105 with additional N- and C-terminal flanking sequences of the corresponding sequence of SEQ ID NO. 61.

6. A MHC class II antigenic peptide comprising
(a) the amino acid sequence of the peptide binding motif of SEQ ID NO. 106, or
(b) at least the amino acid sequence of the peptide binding motif of SEQ ID NO. 106 with additional N- and C-terminal flanking sequences of the corresponding sequence of SEQ ID NO. 62.

7. A MHC class II antigenic peptide comprising
(a) at least the amino acid sequence of the peptide binding motif of SEQ ID NO. 107, or
(b) the amino acid sequence of the peptide binding motif of SEQ ID NO. 107 with additional N- and C-terminal flanking sequences of the corresponding sequence of SEQ ID NO. 63.

8. A MHC class II antigenic peptide comprising
(a) the amino acid sequence of the peptide binding motif of SEQ ID NO. 50, or
(b) the amino acid sequence of the peptide binding motif of SEQ ID NO. 50 with additional N- and C-terminal flanking sequences of the corresponding sequence of SEQ ID NO. 5.

9. A MHC class II antigenic peptide comprising
(a) at least the amino acid sequence of the peptide binding motif of SEQ ID NO. 108, or
(b) the amino acid sequence of the peptide binding motif of SEQ ID NO. 108 with additional N- and C-terminal flanking sequences of the corresponding sequence of SEQ ID NOs. 64 to 67.

10. A MHC class II antigenic peptide comprising
(a) the amino acid sequence of the peptide binding motif of SEQ ID NO. 109, or
(b) at least the amino acid sequence of the peptide binding motif of SEQ ID NO. 109 with additional N- and C-terminal flanking sequences of the corresponding sequence of SEQ ID NO. 68.

11. A MHC class II antigenic peptide comprising
(a) the amino acid sequence of the peptide binding motif of SEQ ID NO. 110, or
(b) the amino acid sequence of the peptide binding motif of SEQ ID NO. 110 with additional N- and C-terminal flanking sequences of the corresponding sequence of SEQ ID NOs. 69 and 70.

12. A MHC class II antigenic peptide comprising
(a) the amino acid sequence of the peptide binding motif of SEQ ID NO. 111, or
(b) the amino acid sequence of the peptide binding motif of SEQ ID NO. 111 with additional N- and C-terminal flanking sequences of the corresponding sequence of SEQ ID NO. 72.

13. A MHC class II antigenic peptide comprising
(a) the amino acid sequence of the peptide binding motif of SEQ ID NO. 112, or
(b) the amino acid sequence of the peptide binding motif of SEQ ID NO. 112 with additional N- and C-terminal flanking sequences of the corresponding sequence of SEQ ID NO. 73.

14. The MHC class II antigenic peptide of claim 1 inked to a MHC class II molecule.

15. A purified antibody composition which is selectively reactive to a MHC class II antigenic peptide according to claim 1.

16. A nucleic acid molecule encoding a peptide according to claim 1.

17. A recombinant nucleic acid construct comprising the nucleic acid molecule according to claim 16 operably linked to an expression vector.

18. A host cell containing the nucleic acid construct according to claim 17.

19-22. (canceled)

23. A pharmaceutical composition comprising a MHC class II antigenic peptide according to claim 1 and a pharmaceutically acceptable carrier.

24. (canceled)

25. A method for diagnosing RA comprising detecting in a patient serum sample the presence of one or more peptides according to claim 1.

26. (canceled)

27. (canceled)

28. A method for diagnosing RA comprising detecting in a patient serum sample the presence of one or more peptides selected from the group consisting of SEQ ID NOs 40 to 48 and SEQ. ID NOs 123 to 141.

29. (canceled)

30. A pharmaceutical composition comprising a peptide selected from the group consisting of SEQ ID NOs 40 to 48 and SEQ ID NOs. 123 to 141, and a pharmaceutically acceptable carrier.

31. A pharmaceutical composition comprising an antibody according to claim 15 and a pharmaceutically acceptable carrier.


SEQUENCE LISTING (141 sequences)

SEQ ID NO: 1 - protein, 14 aa, Homo sapiens
GDRGMQLMHANAQR

SEQ ID NO: 2 - protein, 17 aa, Homo sapiens
GDRGMQLMHANAQRTDA

SEQ ID NO: 3 - protein, 16 aa, Homo sapiens
GDRGMQLMHANAQRTD

SEQ ID NO: 4 - protein, 16 aa, Homo sapiens
INNQLTLDSNTKYFHK

SEQ ID NO: 5 - protein, 17 aa, Homo sapiens
INNQLTLDSNTKYFHKL

SEQ ID NO: 6 - protein, 19 aa, Homo sapiens
MPKNVVFVIDKSGSMSGRK

SEQ ID NO: 7 - protein, 18 aa, Homo sapiens
MPKNVVFVIDKSGSMSGR

SEQ ID NO: 8 - protein, 17 aa, Homo sapiens
MPKNVVFVIDKSGSMSG

SEQ ID NO: 9 - protein, 14 aa, Homo sapiens
NVVFVIDKSGSMSG

SEQ ID NO: 10 - protein, 15 aa, Homo sapiens
KNVVFVIDKSGSMSG

SEQ ID NO: 11 - protein, 15 aa, Homo sapiens
NVVFVIDKSGSMSGR

SEQ ID NO: 12 - protein, 16 aa, Homo sapiens
NVVFVIDKSGSMSGRK

SEQ ID NO: 13 - protein, 15 aa, Homo sapiens
GHPQYLLDSNSWIEE

SEQ ID NO: 14 - protein, 16 aa, Homo sapiens
GHPQYLLDSNSWIEEM

SEQ ID NO: 15 - protein, 14 aa, Homo sapiens
GHPQYLLDSNSWIE

SEQ ID NO: 16 - protein, 18 aa, Homo sapiens
GHPQYLLDSNSWIEEMPS

SEQ ID NO: 17 - protein, 13 aa, Homo sapiens
HPQYLLDSNSWIE

SEQ ID NO: 18 - protein, 12 aa, Homo sapiens
GHPQYLLDSNSW

SEQ ID NO: 19 - protein, 19 aa, Homo sapiens
GVDRYISKYELDKAFSDRN

SEQ ID NO: 20 - protein, 15 aa, Homo sapiens
RYISKYELDKAFSDR

SEQ ID NO: 21 - protein, 14 aa, Homo sapiens
ISKYELDKAFSDRN

SEQ ID NO: 22 - protein, 15 aa, Homo sapiens
ISKYELDKAFSDRNT

SEQ ID NO: 23 - protein, 13 aa, Homo sapiens
ISKYELDKAFSDR

SEQ ID NO: 24 - protein, 16 aa, Homo sapiens
GSREIKSQQSEVTRIL

SEQ ID NO: 25 - protein, 12 aa, Homo sapiens
REIKSQQSEVTR

SEQ ID NO: 26 - protein, 14 aa, Homo sapiens
GSREIKSQQSEVTR

SEQ ID NO: 27 - protein, 14 aa, Homo sapiens
REIKSQQSEVTRIL

SEQ ID NO: 28 - protein, 17 aa, Homo sapiens
GPHDVHVQIETSPPARN

SEQ ID NO: 29 - protein, 19 aa, Homo sapiens
GPHDVHVQIETSPPARNLK

SEQ ID NO: 30 - protein, 16 aa, Homo sapiens
GPHDVHVQIETSPPAR

SEQ ID NO: 31 - protein, 18 aa, Homo sapiens
TPHGIILDSVDAAFICPG

SEQ ID NO: 32 - protein, 17 aa, Homo sapiens
TPHGIILDSVDAAFICP

SEQ ID NO: 33 - protein, 13 aa, Homo sapiens
TPHGIILDSVDAA

SEQ ID NO: 34 - protein, 14 aa, Homo sapiens
GTPHGIILDSVDAA

SEQ ID NO: 35 - protein, 15 aa, Homo sapiens
TPHGIILDSVDAAFI

SEQ ID NO: 36 - protein, 18 aa, Homo sapiens
IDKEGVIEPDTDAPQEMG

SEQ ID NO: 37 - protein, 15 aa, Homo sapiens
KEGVIEPDTDAPQEM

SEQ ID NO: 38 - protein, 16 aa, Homo sapiens
IDKEGVIEPDTDAPQE

SEQ ID NO: 39 - protein, 15 aa, Homo sapiens
DKEGVIEPDTDAPQE

SEQ ID NO: 40 - protein, 261 aa, Homo sapiens [sequence omitted]

SEQ ID NO: 41 - protein, 4563 aa, Homo sapiens
  ref <308>: DATABASE ACCESSION NUMBER: Swiss-Prot/P04114
  ref <309>: DATABASE ENTRY DATE: 1986-11-01
  ref <313>: RELEVANT RESIDUES IN SEQ ID NO: (1)..(4563)
MDPPRPALLALLALPALLLLLLAGARAEEEMLENVSLVCPKDATRFKHLRKYTYNYEAESSSGVPGTADSRSATRINCKVELEVPQLCSFILKTSQCTLKEVYGFNPEGKALLKKTKNSEEFAAAMSRYELKLAIPEGKQVFLYPEKDEPTYILNIKRGIISALLVPPETEEAKQVLFLDTVYGNCSTHFTVKTRKGNVATEISTERDLGQCDRFKPIRTGISPLALIKGMTRPLSTLISSSQSCQYTLDAKRKHVAEAICKEQHLFLPFSYNNKYGMVAQVTQTLKLEDTPKINSRFFGEGTKKMGLAFESTKSTSPPKQAEAVLKTLQELKKLTISEQNIQRANLFNKLVTELRGLSDEAVTSLLPQLIEVSSPITLQALVQCGQPQCSTHILQWLKRVHANPLLIDVVTYLVALIPEPSAQQLREIFNMARDQRSRATLYALSHAVNNYHKTNPTGTQELLDIANYLMEQIQDDCTGDEDYTYLILRVIGNMGQTMEQLTPELKSSILKCVQSTKPSLMIQKAAIQALRKMEPKDKDQEVLLQTFLDDASPGDKRLAAYLMLMRSPSQADINKIVQILPWEQNEQVKNFVASHIANILNSEELDIQDLKKLVKEALKESQLPTVMDFRKFSRNYQLYKSVSLPSLDPASAKIEGNLIFDPNNYLPKESMLKTTLTAFGFASADLIEIGLEGKGFEPTLEALFGKQGFFPDSVNKALYWVNGQVPDGVSKVLVDHFGYTKDDKHEQDMVNGIMLSVEKLIKDLKSKEVPEARAYLRILGEELGFASLHDLQLLGKLLLMGARTLQGIPQMIGEVIRKGSKNDFFLHYIFMENAFELPTGAGLQLQISSSGVIAPGAKAGVKLEVANMQAELVAKPSVSVEFVTNMGIIIPDFARSGVQMNTNFFHESGLEAHVALKAGKLKFIIPSPKRPVKLLSGGNTLHLVSTTKTEVIPPLIENRQSWSVCKQVFPGLNYCTSGAYSNASSTDSASYYPLTGDTRLELELRPTGEIEQYSVSATYELQREDRALVDTLKFVTQAEGAKQTEATMTFKYNRQSMTLSSEVQIPDFDVDLGTILRVNDESTEGKTSYRLTLDIQNKKITEVALMGHLSCDTKEERKIKGVISIPRLQAEARSEILAHWSPAKLLLQMDSSATAYGSTVSKRVAWHYDEEKIEFEWNTGTNVDTKKMTSNFPVDLSDYPKSLHMYANRLLDHRVPETDMTFRHVGSKLIVAMSSWLQKASGSLPYTQTLQDHLNSLKEFNLQNMGLPDFHIPENLFLKSDGRVKYTLNKNSLKIEIPLPFGGKSSRDLKMLETVRTPALHFKSVGFHLPSREFQVPTFTIPKLYQLQVPLLGVLDLSTNVYSNLYNWSASYSGGNTSTDHFSLRARYHMKADSVVDLLSYNVQGSGETTYDHKNTFTLSCDGSLRHKFLDSNIKFSHVEKLGNNPVSKGLLIFDASSSWGPQMSASVHLDSKKKQHLFVKEVKIDGQFRVSSFYAKGTYGLSCQRDPNTGRLNGESNLRFNSSYLQGTNQITGRYEDGTLSLTSTSDLQSGIIKNTASLKYENYELTLKSDTNGKYKNFATSNKMDMTFSKQNALLRSEYQADYESLRFFSLLSGSLNSHGLELNADILGTDKINSGAHKATLRIGQDGISTSATTNLKCSLLVLENELNAELGLSGASMKLTTNGRFREHNAKFSLDGKAALTELSLGSAYQAMILGVDSKNIFNFKVSQEGLKLSNDMMGSYAEMKFDHTNSLNIAGLSLDFSSKLDNIYSSDKFYKQTVNLQLQPYSLVTTLNSDLKYNALDLTNNGKLRLEPLKLHVAGNLKGAYQNNEIKHIYAISSAALSASYKADTVAKVQGVEFSHRLNTDIAGLASAIDMSTNYNSDSLHFSNVFRSVMAPFTMTIDAHTNGNGKLALWGEHTGQLYSKFLLKAEPLAFTFSHDYKGSTSHHLVSRKSISAALEHKVSALLTPAEQTGTWKLKTQFNNNEYSQDLDAYNTKDKIGVELTGRTLADLTLLDSPIKVPLLLSEPINIIDALEMRDAVEKPQEFTIVAFVKYDKNQDVHSINLPFFETLQEYFERNRQTIIVVVENVQRNLKHINIDQFVRKYRAALGKLPQQANDYLNSFNWERQVSHAKEKLTALTKKYRITENDIQIALDDAKINFNEKLSQLQTYMIQFDQYIKDSYDLHDLKIAIANIIDEIIEKLKSLDEHYHIRVNLVKTIHDLHLFIENIDFNKSGSSTASWIQNVDTKYQIRIQIQEKLQQLKRHIQNIDIQHLAGKLKQHIEAIDVRVLLDQLGTTISFERINDVLEHVKHFVINLIGDFEVAEKINAFRAKVHELIERYEVDQQIQVLMDKLVELTHQYKLKETIQKLSNVLQQVKIKDYFEKLVGFIDDAVKKLNELSFKTFIEDVNKFLDMLIKKLKSFDYHQFVDETNDKIREVTQRLNGEIQALELPQKAEALKLFLEETKATVAVYLESLQDTKITLIINWLQEALSSASLAHMKAKFRETLEDTRDRMYQMDIQQELQRYLSLVGQVYSTLVTYISDWWTLAAKNLTDFAEQYSIQDWAKRMKALVEQGFTVPEIKTILGTMPAFEVSLQALQKATFQTPDFIVPLTDLRIPSVQINFKDLKNIKIPSRFSTPEFTILNTFHIPSFTIDFVEMKVKIIRTIDQMQNSELQWPVPDIYLRDLKVEDIPLARITLPDFRLPEIAIPEFIIPTLNLNDFQVPDLHIPEFQLPHISHTIEVPTFGKLYSILKIQSPLFTLDANADIGNGTTSANEAGIAASITAKGESKLEVLNFDFQANAQLSNPKINPLALKESVKFSSKYLRTEHGSEMLFFGNAIEGKSNTVASLHTEKNTLELSNGVIVKINNQLTLDSNTKYFHKLNIPKLDFSSQADLRNEIKTLLKAGHIAWTSSGKGSWKWACPRFSDEGTHESQISFTIEGPLTSFGLSNKINSKHLRVNQNLVYESGSLNFSKLEIQSQVDSQHVGHSVLTAKGMALFGEGKAEFTGRHDAHLNGKVIGTLKNSLFFSAQPFEITASTNNEGNLKVRFPLRLTGKIDFLNNYALFLSPSAQQASWQVSARFNQYKYNQNFSAGNNENIMEAHVGINGEANLDFLNIPLTIPEMRLPYTIITTPPLKDFSLWEKTGLKEFLKTTKQSFDLSVKAQYKKNKHRHSITNPLAVLCEFISQSIKSFDRHFEKNRNNALDFVTKSYNETKIKFDKYKAEKSHDELPRTFQIPGYTVPVVNVEVSPFTIEMSAFGYVFPKAVSMPSFSILGSDVRVPSYTLILPSLELPVLHVPRNLKLSLPHFKELCTISHIFIPAMGNITYDFSFKSSVITLNTNAELFNQSDIVAHLLSSSSSVIDALQYKLEGTTRLTRKRGLKLATALSLSNKFVEGSHNSTVSLTTKNMEVSVAKTTKAEIPILRMNFKQELNGNTKSKPTVSSSMEFKYDFNSSMLYSTAKGAVDHKLSLESLTSYFSIESSTKGDVKGSVLSREYSGTIASEANTYLNSKSTRSSVKLQGTSKIDDIWNLEVKENFAGEATLQRIYSLWEHSTKNHLQLEGLFFTNGEHTSKATLELSPWQMSALVQVHASQPSSFHDFPDLGQEVALNANTKNQKIRWKNEVRIHSGSFQSQVELSNDQEKAHLDIAGSLEGHLRFLKNIILPVYDKSLWDFLKLDVTTSIGRRQHLRVSTAFVYTKNPNGYSFSIPVKVLADKFITPGLKLNDLNSVLVMPTFHVPFTDLQVPSCKLDFREIQIYKKLRTSSFALNLPTLPEVKFPEVDVLTKYSQPEDSLIPFFEITVPESQLTVSQFTLPKSVSDGIAALDLNAVANKIADFELPTIIVPEQTIEIPSIKFSVPAGIVIPSFQALTARFEVDSPVYNATWSASLKNKADYVETVLDSTCSSTVQFLEYELNVLGTHKIEDGTLASKTKGTLAHRDFSAEYEEDGKFEGLQEWEGKAHLNIKSPAFTDLHLRYQKDKKGISTSAASPAVGTVGMDMDEDDDFSKWNFYYSPQSSPDKKLTIFKTELRVRESDEETQIKVNWEEEAASGLLTSLKDNVPKATGVLYDYVNKYHWEHTGLTLREVSSKLRRNLQNNAEWVYQGAIRQIDDIDVRFQKAASGTTGTYQEWKDKAQNLYQELLTQEGQASFQGLKDNVFDGLVRVTQKFHMKVKHLIDSLIDFLNFPRFQFPGKPGIYTREELCTMFIREVGTVLSQVYSKVHNGSEILFSYFQDLVITLPFELRKHKLIDVISMYRELLKDLSKEAQEVFKAIQSLKTTEVLRNLQDLLQFIFQLIEDNIKQLKEMKFTYLINYIQDEINTIFNDYIPYVFKLLKENLCLNLHKFNEFIQNELQEASQELQQIHQYIMALREEYFDPSIVGWTVKYYELEEKIVSLIKNLLVALKDFHSEYIVSASNFTSQLSSQVEQFLHRNIQEYLSILTDPDGKGKEKIAELSATAQEIIKSQAIATKKIISDYHQQFRYKLQDFSDQLSDYYEKFIAESKRLIDLSIQNYHTFLIYITELLKKLQSTTVMNPYMKLAPGELTIIL

SEQ ID NO: 42 - protein, 930 aa, Homo sapiens [sequence omitted]

SEQ ID NO: 43 - protein, 1744 aa, Homo sapiens
  ref <308>: DATABASE ACCESSION NUMBER: Swiss-Prot/P01028
  ref <309>: DATABASE ENTRY DATE: 1986-07-21
  ref <313>: RELEVANT RESIDUES IN SEQ ID NO: (1)..(1744)
MRLLWGLIWASSFFTLSLQKPRLLLFSPSVVHLGVPLSVGVQLQDVPRGQVVKGSVFLRNPSRNNVPCSPKVDFTLSSERDFALLSLQVPLKDAKSCGLHQLLRGPEVQLVAHSPWLKDSLSRTTNIQGINLLFSSRRGHLFLQTDQPIYNPGQRVRYRVFALDQKMRPSTDTITVMVENSHGLRVRKKEVYMPSSIFQDDFVIPDISEPGTWKISARFSDGLESNSSTQFEVKKYVLPNFEVKITPGKPYILTVPGHLDEMQLDIQARYIYGKPVQGVAYVRFGLLDEDGKKTFFRGLESQTKLVNGQSHISLSKAEFQDALEKLNMGITDLQGLRLYVAAAIIESPGGEMEEAELTSWYFVSSPFSLDLSKTKRHLVPGAPFLLQALVREMSGSPASGIPVKVSATVSSPGSVPEVQDIQQNTDGSGQVSIPIIIPQTISELQLSVSAGSPHPAIARLTVAAPPSGGPGFLSIERPDSRPPRVGDTLNLNLRAVGSGATFSHYYYMILSRGQIVFMNREPKRTLTSVSVFVDHHLAPSFYFVAFYYHGDHPVANSLRVDVQAGACEGKLELSVDGAKQYRNGESVKLHLETDSLALVALGALDTALYAAGSKSHKPLNMGKVFEAMNSYDLGCGPGGGDSALQVFQAAGLAFSDGDQWTLSRKRLSCPKEKTTRKKRNVNFQKAINEKLGQYASPTAKRCCQDGVTRLPMMRSCEQRAARVQQPDCREPFLSCCQFAESLRKKSRDKGQAGLQRALEILQEEDLIDEDDIPVRSFFPENWLWRVETVDRFQILTLWLPDSLTTWEIHGLSLSKTKGLCVATPVQLRVFREFHLHLRLPMSVRRFEQLELRPVLYNYLDKNLTVSVHVSPVEGLCLAGGGGLAQQVLVPAGSARPVAFSVVPTAAAAVSLKVVARGSFEFPVGDAVSKVLQIEKEGAIHREELVYELNPLDHRGRTLEIPGNSDPNMIPDGDFNSYVRVTASDPLDTLGSEGALSPGGVASLLRLPRGCGEQTMIYLAPTLAASRYLDKTEQWSTLPPETKDHAVDLIQKGYMRIQQFRKADGSYAAWLSRDSSTWLTAFVLKVLSLAQEQVGGSPEKLQETSNWLLSQQQADGSFQDPCPVLDRSMQGGLVGNDETVALTAFVTIALHHGLAVFQDEGAEPLKQRVEASISKANSFLGEKASAGLLGAHAAAITAYALSLTKAPVDLLGVAHNNLMAMAQETGDNLYWGSVTGSQSNAVSPTPAPRNPSDPMPQAPALWIETTAYALLHLLLHEGKAEMADQASAWLTRQGSFQGGFRSTQDTVIALDALSAYWIASHTTEERGLNVTLSSTGRNGFKSHALQLNNRQIRGLEEELQFSLGSKINVKVGGNSKGTLKVLRTYNVLDMKNTTCQDLQIEVTVKGHVEYTMEANEDYEDYEYDELPAKDDPDAPLQPVTPLQLFEGRRNRRRREAPKVVEEQESRVHYTVCIWRNGKVGLSGMAIADVTLLSGFHALRADLEKLTSLSDRYVSHFETEGPHVLLYFDSVPTSRECVGFEAVQEVPVGLVQPASATLYDYYNPERRCSVFYGAPSKSRLLATLCSAEVCQCAEGKCPRQRRALERGLQDEDGYRMKFACYYPRVEYGFQVKVLREDSRAAFRLFETKITQVLHFTKDVKAAANQMRNFLVRASCRLRLEPGKEYLIMGLDGATYDLEGHPQYLLDSNSWIEEMPSERLCRSTRQRAACAQLNDFLQEYGTQGCQV

SEQ ID NO: 44 - protein, 1663 aa, Homo sapiens
  ref <308>: DATABASE ACCESSION NUMBER: Swiss-Prot/P01024
  ref <309>: DATABASE ENTRY DATE: 1986-07-21
  ref <313>: RELEVANT RESIDUES IN SEQ ID NO: (1)..(1663)
MGPTSGPSLLLLLLTHLPLALGSPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGYLFIQTDKTIYTPGSTVLYRIFTVNHKLLPVGRTVMVNIENPEGIPVKQDSLSSQNQLGVLPLSWDIPELVNMGQWKIRAYYENSPQQVFSTEFEVKEYVLPSFEVIVEPTEKFYYIYNEKGLEVTITARFLYGKKVEGTAFVIFGIQDGEQRISLPESLKRIPIEDGSGEVVLSRKVLLDGVQNLRAEDLVGKSLYVSATVILHSGSDMVQAERSGIPIVTSPYQIHFTKTPKYFKPGMPFDLMVFVTNPDGSPAYRVPVAVQGEDTVQSLTQGDGVAKLSINTHPSQKPLSITVRTKKQELSEAEQATRTMQALPYSTVGNSNNYLHLSVLRTELRPGETLNVNFLLRMDRAHEAKIRYYTYLIMNKGRLLKAGRQVREPGQDLVVLPLSITTDFIPSFRLVAYYTLIGASGQREVVADSVWVDVKDSCVGSLVVKSGQSEDRQPVPGQQMTLKIEGDHGARVVLVAVDKGVFVLNKKNKLTQSKIWDVVEKADIGCTPGSGKDYAGVFSDAGLTFTSSSGQQTAQRAELQCPQPAARRRRSVQLTEKRMDKVGKYPKELRKCCEDGMRENPMRFSCQRRTRFISLGEACKKVFLDCCNYITELRRQHARASHLGLARSNLDEDIIAEENIVSRSEFPESWLWNVEDLKEPPKNGISTKLMNIFLKDSITTWEILAVSMSDKKGICVADPFEVTVMQDFFIDLRLPYSVVRNEQVEIRAVLYNYRQNQELKVRVELLHNPAFCSLATTKRRHQQTVTIPPKSSLSVPYVIVPLKTGLQEVEVKAAVYHHFISDGVRKSLKVVPEGIRMNKTVAVRTLDPERLGREGVQKEDIPPADLSDQVPDTESETRILLQGTPVAQMTEDAVDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSRSSKITHRIHWESASLLRSEETKENEGFTVTAEGKGQGTLSVVTMYHAKAKDQLTCNKFDLKVTIKPAPETEKRPQDAKNTMILEICTRYRGDQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHPEKEDGKLNKLCRDELCRCAEENCFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPNLSYIIGKDTWVEHWPEEDECQDEENQKQCQDLGAFTESMVVFGCPN

SEQ ID NO: 45 - protein, 93 aa, Homo sapiens
  ref <308>: DATABASE ACCESSION NUMBER: Swiss-Prot/Q9H299
  ref <309>: DATABASE ENTRY DATE: 2003-02-28
  ref <313>: RELEVANT RESIDUES IN SEQ ID NO: (1)..(93)
MSGLRVYSTSVTGSREIKSQQSEVTRILDGKRIQYQLVDISQDNALRDEMRALAGNPKATPPQIVNGDQYCGDYELFVEAVEQNTLQEFLKLA

SEQ ID NO: 46 - protein, 567 aa, Homo sapiens
  ref <308>: DATABASE ACCESSION NUMBER: Swiss-Prot/Q96RQ9
  ref <309>: DATABASE ENTRY DATE: 2003-02-28
  ref <313>: RELEVANT RESIDUES IN SEQ ID NO: (1)..(567)
MAPLALHLLVLVPILLSLVASQDWKAERSQDPFEKCMQDPDYEQLLKVVTWGLNRTLKPQRVIVVGAGVAGLVAAKVLSDAGHKVTILEADNRIGGRIFTYRDQNTGWIGELGAMRMPSSHRILHKLCQGLGLNLTKFTQYDKNTWTEVHEVKLRNYVVEKVPEKLGYALRPQEKGHSPEDIYQMALNQALKDLKALGCRKAMKKFERHTLLEYLLGEGNLSRPAVQLLGDVMSEDGFFYLSFAEALRAHSCLSDRLQYSRIVGGWDLLPRALLSSLSGLVLLNAPVVAMTQGPHDVHVQIETSPPARNLKVLKADVVLLTASGPAVKRITFSPPLPRHMQEALRRLHYVPATKVFLSFRRPFWREEHIEGGHSNTDRPSRMIFYPPPREGALLLASYTWSDAAAAFAGLSREEALRLALDDVAALHGPVVRQLWDGTGVVKRWAEDQHSQGGFVVQPPALWQTEKDDWTVPYGRIYFAGEHTAYPHGWVETAVKSALRAAIKINSRKGPASDTASPEGHASDMEGQGHVHGVASSPSHDLAKEEGSHPPVQGQLSLQNTTHTRTSH

SEQ ID NO: 47 - protein, 462 aa, Homo sapiens
  ref <308>: DATABASE ACCESSION NUMBER: Swiss-Prot/P02790
  ref <309>: DATABASE ENTRY DATE: 1986-07-21
  ref <313>: RELEVANT RESIDUES IN SEQ ID NO: (1)..(462)
MARVLGAPVALGLWSLCWSLAIATPLPPTSAHGNVAEGETKPDPDVTERCSDGWSFDATTLDDNGTMLFFKGEFVWKSHKWDRELISERWKNFPSPVDAAFRQGHNSVFLIKGDKVWVYPPEKKEKGYPKLLQDEFPGIPSPLDAAVECHRGECQAEGVLFFQGDREWFWDLATGTMKERSWPAVGNCSSALRWLGRYYCFQGNQFLRFDPVRGEVPPRYPRDVRDYFMPCPGRGHGHRNGTGHGNSTHHGPEYMRCSPHLVLSALTSDNHGATYAFSGTHYWRLDTSRDGWHSWPIAHQWPQGPSAVDAAFSWEEKLYLVQGTQVYVFLTKGGYTLVSGYPKRLEKEVGTPHGIILDSVDAAFICPGSSRLHIMAGRRLWWLDLKSGAQATWTELPWPHEKVDGALCMEKSLGPNSCSANGPGLYLIHGPNLYCYSDVEKLNAAKALPQPQNVTSLLGCTH

SEQ ID NO: 48 - protein, 369 aa, Homo sapiens
  ref <308>: DATABASE ACCESSION NUMBER: Swiss-Prot/P50502
  ref <309>: DATABASE ENTRY DATE: 1996-10-02
  ref <313>: RELEVANT RESIDUES IN SEQ ID NO: (1)..(369)
MDPRKVNELRAFVKMCKQDPSVLHTEEMRFLREWVESMGGKVPPATQKAKSEENTKEEKPDSKKVEEDLKADEPSSEESDLEIDKEGVIEPDTDAPQEMGDENAEITEEMMDQANDKKVAAIEALNDGELQKAIDLFTDAIKLNPRLAILYAKRASVFVKLQKPNAAIRDCDRAIEINPDSAQPYKWRGKAHRLLGHWEEAAHDLALACKLDYDEDASAMLKEVQPRAQKIAEHRRKYERKREEREIKERIERVKKAREEHERAQREEEARRQSGAQYGSFPGGFPGGMPGNFPGGMPGMGGGMPGMAGMPGLNEILSDPEVLAAMQDPEVMVAFQDVAQNPANMSKYQSNPKVMNLISKLSAKFGGQA

SEQ ID NO: 49 - protein, 9 aa, Homo sapiens
MQLMHANAQ

SEQ ID NO: 50 - protein, 9 aa, Homo sapiens
LTLDSNTKY

SEQ ID NO: 51 - protein, 9 aa, Homo sapiens
FVIDKSGSM

SEQ ID NO: 52 - protein, 9 aa, Homo sapiens
YLLDSNSWI

SEQ ID NO: 53 - protein, 9 aa, Homo sapiens
YELDKAFSD

SEQ ID NO: 54 - protein, 9 aa, Homo sapiens
IKSQQSEVT

SEQ ID NO: 55 - protein, 9 aa, Homo sapiens
VQIETSPPA

SEQ ID NO: 56 - protein, 9 aa, Homo sapiens
IILDSVDAA

SEQ ID NO: 57 - protein, 9 aa, Homo sapiens
IEPDTDAPQ

SEQ ID NO: 58 - protein, 17 aa, Homo sapiens
NIQPIFAVTSRMVKTYE

SEQ ID NO: 59 - protein, 19 aa, Homo sapiens
ENNIQPIFAVTSRMVKTYE

SEQ ID NO: 60 - protein, 17 aa, Homo sapiens
NKVFGEDSVGVIFKNGD

SEQ ID NO: 61 - protein, 16 aa, Homo sapiens
YPEQLKMTVVKLISHR

SEQ ID NO: 62 - protein, 16 aa, Homo sapiens
KNTLYLQMNSLRAEDT

SEQ ID NO: 63 - protein, 16 aa, Homo sapiens
NGGHYTYSENRVEKDG

SEQ ID NO: 64 - protein, 15 aa, Homo sapiens
GPNNYYSFASQQQKP

SEQ ID NO: 65 - protein, 16 aa, Homo sapiens
GPNNYYSFASQQQKPE

SEQ ID NO: 66 - protein, 17 aa, Homo sapiens
GPNNYYSFASQQQKPED

SEQ ID NO: 67 - protein, 18 aa, Homo sapiens
GPNNYYSFASQQQKPEDT

SEQ ID NO: 68 - protein, 16 aa, Homo sapiens
EKLWFVPAKVEDSGHY

SEQ ID NO: 69 - protein, 14 aa, Homo sapiens
SLRELHLDHNQISR

SEQ ID NO: 70 - protein, 13 aa, Homo sapiens
LRELHLDHNQISR

SEQ ID NO: 71 - protein, 15 aa, Homo sapiens
ETMKMRYEHIDHTFE

SEQ ID NO: 72 - protein, 17 aa, Homo sapiens
ETMKMRYEHIDHTFEIQ

SEQ ID NO: 73 - protein, 16 aa, Homo sapiens
HMFLQDEIIDKSYTPS

SEQ ID NO: 74 - protein, 17 aa, Homo sapiens
VDRYISKYELDKAFSDR

SEQ ID NO: 75 - protein, 14 aa, Homo sapiens
RYISKYELDKAFSD

SEQ ID NO: 76 - protein, 19 aa, Homo sapiens
LPVGRTVMVNIENPEGIPV

SEQ ID NO: 77 - protein, 20 aa, Homo sapiens
LPVGRTVMVNIENPEGIPVK

SEQ ID NO: 78 - protein, 18 aa, Homo sapiens
GTPHGIILDSVDAAFICP

SEQ ID NO: 79 - protein, 18 aa, Homo sapiens
MATPLLMQALPMGALPQG

SEQ ID NO: 80 - protein, 17 aa, Homo sapiens
MATPLLMQALPMGALPQ

SEQ ID NO: 81 - protein, 16 aa, Homo sapiens
MATPLLMQALPMGALP

SEQ ID NO: 82 - protein, 21 aa, Homo sapiens
MATPLLMQALPMGALPQGPMQ

SEQ ID NO: 83 - protein, 15 aa, Homo sapiens
ATPLLMQALPMGALP

SEQ ID NO: 84 - protein, 23 aa, Homo sapiens
HPPVQWAFQETSVESAVDTPFPA

SEQ ID NO: 85 - protein, 24 aa, Homo sapiens
HPPVQWAFQETSVESAVDTPFPAG

SEQ ID NO: 86 - protein, 22 aa, Homo sapiens
HPPVQWAFQETSVESAVDTPFP

SEQ ID NO: 87 - protein, 17 aa, Homo sapiens
KIYLYTLNDNARSSPVV

SEQ ID NO: 88 - protein, 16 aa, Homo sapiens
KIYLYTLNDNARSSPV

SEQ ID NO: 89 - protein, 17 aa, Homo sapiens
IYLYTLNDNARSSPVVI

SEQ ID NO: 90 - protein, 15 aa, Homo sapiens
IYLYTLNDNARSSPV

SEQ ID NO: 91 - protein, 16 aa, Homo sapiens
IYLYTLNDNARSSPVV

SEQ ID NO: 92 - protein, 15 aa, Homo sapiens
YNSYSVSNSEKDIMA

SEQ ID NO: 93 - protein, 15 aa, Homo sapiens
AGSLTLSKTELGKKA

SEQ ID NO: 94 - protein, 16 aa, Homo sapiens
AGSLTLSKTELGKKAD

SEQ ID NO: 95 - protein, 14 aa, Homo sapiens
VPKDYTGEDVTPQN

SEQ ID NO: 96 - protein, 13 aa, Homo sapiens
DSKFHQAINDAHQ

SEQ ID NO: 97 - protein, 15 aa, Homo sapiens
MPLEFKTLNVLHNRG

SEQ ID NO: 98 - protein, 15 aa, Homo sapiens
ATRSIQVDGKTIKAQ

SEQ ID NO: 99 - protein, 18 aa, Homo sapiens
ATRSIQVDGKTIKAQIWD

SEQ ID NO: 100 - protein, 14 aa, Homo sapiens
TRSIQVDGKTIKAQ

SEQ ID NO: 101 - protein, 17 aa, Homo sapiens
ATRSIQVDGKTIKAQIW

SEQ ID NO: 102 - protein, 13 aa, Homo sapiens
RSIQVDGKTIKAQ

SEQ ID NO: 103 - protein, 9 aa, Homo sapiens
IQPIFAVTS

SEQ ID NO: 104 - protein, 9 aa, Homo sapiens
VFGEDSVGV

SEQ ID NO: 105 - protein, 9 aa, Homo sapiens
LKMTVVKLI

SEQ ID NO: 106 - protein, 9 aa, Homo sapiens
YLQMNSLRA

SEQ ID NO: 107 - protein, 9 aa, Homo sapiens
YTYSENRVE

SEQ ID NO: 108 - protein, 9 aa, Homo sapiens
YYSFASQQQ

SEQ ID NO: 109 - protein, 9 aa, Homo sapiens
FVPAKVEDS

SEQ ID NO: 110 - protein, 9 aa, Homo sapiens
LHLDHNQIS

SEQ ID NO: 111 - protein, 9 aa, Homo sapiens
MRYEHIDHT

SEQ ID NO: 112 - protein, 9 aa, Homo sapiens
FLQDEIIDK

SEQ ID NO: 113 - protein, 9 aa, Homo sapiens
MVNIENPEG

SEQ ID NO: 114 - protein, 9 aa, Homo sapiens
LMQALPMGA

SEQ ID NO: 115 - protein, 9 aa, Homo sapiens
WAFQETSVE

SEQ ID NO: 116 - protein, 9 aa, Homo sapiens
YTLNDNARS

SEQ ID NO: 117 - protein, 9 aa, Homo sapiens
YSVSNSEKD

SEQ ID NO: 118 - protein, 9 aa, Homo sapiens
LTLSKTELG

SEQ ID NO: 119 - protein, 9 aa, Homo sapiens
YTGEDVTPQ

SEQ ID NO: 120 - protein, 9 aa, Homo sapiens
FHQAINDAH

SEQ ID NO: 121 - protein, 9 aa, Homo sapiens
FKTLNVLHN

SEQ ID NO: 122 - protein, 9 aa, Homo sapiens
IQVDGKTIK

SEQ ID NO: 123 - protein, 769 aa, Homo sapiens
  ref <308>: DATABASE ACCESSION NUMBER: Swissprot/P05107
  ref <309>: DATABASE ENTRY DATE: 1987-08-13
  ref <313>: RELEVANT RESIDUES IN SEQ ID NO: (1)..(769)
MLGLRPPLLALVGLLSLGCVLSQECTKFKVSSCRECIESGPGCTWCQKLNFTGPGDPDSIRCDTRPQLLMRGCAADDIMDPTSLAETQEDHNGGQKQLSPQKVTLYLRPGQAAAFNVTFRRAKGYPIDLYYLMDLSYSMLDDLRNVKKLGGDLLRALNEITESGRIGFGSFVDKTVLPFVNTHPDKLRNPCPNKEKECQPPFAFRHVLKLTNNSNQFQTEVGKQLISGNLDAPEGGLDAMMQVAACPEEIGWRNVTRLLVFATDDGFHFAGDGKLGAILTPNDGRCHLEDNLYKRSNEFDYPSVGQLAHKLAENNIQPIFAVTSRMVKTYEKLTEIIPKSAVGELSEDSSNVVHLIKNAYNKLSSRVFLDHNALPDTLKVTYDSFCSNGVTHRNQPRGDCDGVQINVPITFQVKVTATECIQEQSFVIRALGFTDIVTVQVLPQCECRCRDQSRDRSLCHGKGFLECGICRCDTGYIGKNCECQTQGRSSQELEGSCRKDNNSIICSGLGDCVCGQCLCHTSDVPGKLIYGQYCECDTINCERYNGQVCGGPGRGLCFCGKCRCHPGFEGSACQCERTTEGCLNPRRVECSGRGRCRCNVCECHSGYQLPLCQECPGCPSPCGKYISCAECLKFEKGPFGKNCSAACPGLQLSNNPVKGRTCKERDSEGCWVAYTLEQQDGMDRYLIYVDESRECVAGPNIAAIVGGTVAGIVLIGILLLVIWKALIHLSDLREYRRFEKEKLKSQWNNDNPLFKSATTTVMNPKFAES

SEQ ID NO: 124 - protein, 1070 aa, Homo sapiens
  ref <308>: DATABASE ACCESSION NUMBER: Swissprot/P42338
  ref <309>: DATABASE ENTRY DATE: 1995-11-01
  ref <313>: RELEVANT RESIDUES IN SEQ ID NO: (1)..(1070)
MCFSFIMPPAMADILDIWAVDSQIASDGSIPVDFLLPTGIYIQLEVPREATISYIKQMLWKQVHNYPMFNLLMDIDSYMFACVNQTAVYEELEDETRRLCDVRPFLPVLKLVTRSCDPGEKLDSKIGVLIGKGLHEFDSLKDPEVNEFRRKMRKFSEEKILSLVGLSWMDWLKQTYPPEHEPSIPENLEDKLYGGKLIVAVHFENCQDVFSFQVSPNMNPIKVNELAIQKRLTIHGKEDEVSPYDYVLQVSGRVEYVFGDHPLIQFQYIRNCVMNRALPHFILVECCKIKKMYEQEMIAIEAAINRNSSNLPLPLPPKKTRIISHVWENNNPFQIVLVKGNKLNTEETVKVHVRAGLFHGTELLCKTIVSSEVSGKNDHIWNEPLEFDINICDLPRMARLCFAVYAVLDKVKTKKSTKTINPSKYQTIRKAGKVHYPVAWVNTMVFDFKGQLRTGDIILHSWSSFPDELEEMLNPMGTVQTNPYTENATALHVKFPENKKQPYYYPPFDKIIEKAAEIASSDSANVSSRGGKKFLPVLKEILDRDPLSQLCENEMDLIWTLRQDCREIFPQSLPKLLLSIKWNKLEDVAQLQALLQIWPKLPPREALELLDFNYPDQYVREYAVGCLRQMSDEELSQYLLQLVQVLKYEPFLDCALSRFLLERALGNRRIGQFLFWHLRSEVHIPAVSVQFGVILEAYCRGSVGHMKVLSKQVEALNKLKTLNSLIKLNAVKLNRAKGKEAMHTCLKQSAYREALSDLQSPLNPCVILSELYVEKCKYMDSKMKPLWLVYNNKVFGEDSVGVIFKNGDDLRQDMLTLQMLRLMDLLWKEAGLDLRMLPYGCLATGDRSGLIEVVSTSETIADIQLNSSNVAAAAAFNKDALLNWLKEYNSGDDLDRAIEEFTLSCAGYCVASYVLGIGDRHSDNIMVKKTGQLFHIDFGHILGNFKSKFGIKRERVPFILTYDFIHVIQQGKTGNTEKFGRFRQCCEDAYLILRRHGNLFITLFALMLTAGLPELTSVKDIQYLKDSLALGKSEEEALKQFKQKFDEALRESWTTKVNWMAHTVRKDYRS

SEQ ID NO: 125 - protein, 431 aa, Homo sapiens [sequence omitted]

SEQ ID NO: 126 - protein, 117 aa, Homo sapiens
  ref <308>: DATABASE ACCESSION NUMBER: Swissprot/P01764
  ref <309>: DATABASE ENTRY DATE: 1986-07-21
  ref <313>: RELEVANT RESIDUES IN SEQ ID NO: (1)..(117)
MEFGLSWLFLVAILKGVQCEVQLLESGGGLVQPGGSLRLSCAASGFTFSSYAMSWVRQAPGKGLEWVSAISGSGGSTYYGDSVKGRFTISRDNSKNTLYLQMNSLRAEDTAVYYCAK

SEQ ID NO: 127 - protein, 189 aa, Homo sapiens
  ref <308>: DATABASE ACCESSION NUMBER: Swissprot/Q99497
  ref <309>: DATABASE ENTRY DATE: 1997-05-01
  ref <313>: RELEVANT RESIDUES IN SEQ ID NO: (1)..(189)
MASKRALVILAKGAEEMETVIPVDVMRRAGIKVTVAGLAGKDPVQCSRDVVICPDASLEDAKKEGPYDVVVLPGGNLGAQNLSESAAVKEILKEQENRKGLIAAICAGPTALLAHEIGFGSKVTTHPLAKDKMMNGGHYTYSENRVEKDGLILTSRGPGTSFEFALAIVEALNGKEVAAQVKAPLVLKD

SEQ ID NO: 128 - protein, 257 aa, Homo sapiens
  ref <308>: DATABASE ACCESSION NUMBER: Swissprot/P48556
  ref <309>: DATABASE ENTRY DATE: 1996-02-01
  ref <313>: RELEVANT RESIDUES IN SEQ ID NO: (1)..(257)
MYEQLKGEWNRKSPNLSKCGEELGRLKLVLLELNFLPTTGTKLTKQQLILARDILEIGAQWSILRKDIPSFERYMAQLKCYYFDYKEQLPESAYMHQLLGLNLLFLLSQNRVAEFHTELERLPAKDIQTNVYIKHPVSLEQYLMEGSYNKVFLAKGNIPAESYTFFIDILLDTIRDEIAGCIEKAYEKILFTEATRILFFNTPKKMTDYAKKRGWVLGPNNYYSFASQQQKPEDTTIPSTELAKQVIEYARQLEMIV

SEQ ID NO: 129 - protein, 569 aa, Homo sapiens
  ref <308>: DATABASE ACCESSION NUMBER: Swissprot/P14778
  ref <309>: DATABASE ENTRY DATE: 1990-04-01
  ref <313>: RELEVANT RESIDUES IN SEQ ID NO: (1)..(569)
MKVLLRLICFIALLISSLEADKCKEREEKIILVSSANEIDVRPCPLNPNEHKGTITWYKDDSKTPVSTEQASRIHQHKEKLWFVPAKVEDSGHYYCVVRNSSYCLRIKISAKFVENEPNLCYNAQAIFKQKLPVAGDGGLVCPYMEFFKNENNELPKLQWYKDCKPLLLDNIHFSGVKDRLIVMNVAEKHRGNYTCHASYTYLGKQYPITRVIEFITLEENKPTRPVIVSPANETMEVDLGSQIQLICNVTGQLSDIAYWKWNGSVIDEDDPVLGEDYYSVENPANKRRSTLITVLNISEIESRFYKHPFTCFAKNTHGIDAAYIQLIYPVTNFQKHMIGICVTLTVIIVCSVFIYKIFKIDIVLWYRDSCYDFLPIKASDGKTYDAYILYPKTVGEGSTSDCDIFVFKVLPEVLEKQCGYKLFIYGRDDYVGEDIVEVINENVKKSRRLIIILVRETSGFSWLGGSSEEQIAMYNALVQDGIKVVLLELEKIQDYEKMPESIKFIKQKHGAIRWSGDFTQGPQSAKTRFWKNVRYHMPVQRRSPSSKHQLLSPATKEKLQREAHVPLG

SEQ ID NO: 130 - protein, 376 aa, Homo sapiens [sequence omitted]

SEQ ID NO: 131 - protein, 897 aa, Homo sapiens
  ref <308>: DATABASE ACCESSION NUMBER: Swissprot/P32927
  ref <309>: DATABASE ENTRY DATE: 1993-10-01
  ref <313>: RELEVANT RESIDUES IN SEQ ID NO: (1)..(897)
MVLAQGLLSMALLALCWERSLAGAEETIPLQTLRCYNDYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTGYNGIWSEWSEARSWDTESVLPMWVLALIVIFLTIAVLLALRFCGIYGYRLRRKWEEKIPNPSKSHLFQNGSAELWPPGSMSAFTSGSPPHQGPWGSRFPELEGVFPVGFGDSEVSPLTIEDPKHVCDPPSGPDTTPAASDLPTEQPPSPQPGPPAASHTPEKQASSFDFNGPYLGPPHSRSLPDILGQPEPPQEGGSQKSPPPGSLEYLCLPAGGQVQLVPLAQAMGPGQAVEVERRPSQGAAGSPSLESGGGPAPPALGPRVGGQDQKDSPVAIPMSSGDTEDPGVASGYVSSADLVFTPNSGASSVSLVPSLGLPSDQTPSLCPGLASGPPGAPGPVKSGFEGYVELPPIEGRSPRSPRNNPVPPEAKSPVLNPGERPADVSPTSPQPEGLLVLQQVGDYCFLPGLGPGPLSLRSKPSSPGPGPEIKNLDQAFQVKKPPGQAVPQVPVIQLFKALKQQDYLSLPPWEVNKPGEVC

SEQ ID NO: 132 - protein, 261 aa, Homo sapiens
  ref <308>: DATABASE ACCESSION NUMBER: Swissprot/Q60493
  ref <309>: DATABASE ENTRY DATE: 1996-11-01
  ref <313>: RELEVANT RESIDUES IN SEQ ID NO: (1)..(261)
MIYKCPMCREFFSERADLFMHQKVHTAEKPHKCDKCDKGFFHISELHIHWRDHTGEKVYKCDDCGKDFSTTTKLNRHKKIHTVEKPYKCYECGKAFNWSPHLQIHMRVHTGEKPYVCSECGRGFSNSSNLCMHQRVHTGEKPFKCEECGKAFRHTSSLCMHQRVHTGEKPYKCYECGKAFSQSSSLCIHQRVHTGEKPYRCCGCGKAFSQSSSLCIHQRVHTGEKPFKCDECGKAFSQSTSLCIHQRVHTKERNHLKISVI

SEQ ID NO: 133 - protein, 296 aa, Homo sapiens
  ref <308>: DATABASE ACCESSION NUMBER: Swissprot/P04233
  ref <309>: DATABASE ENTRY DATE: 1987-03-20
  ref <313>: RELEVANT RESIDUES IN SEQ ID NO: (1)..(296)
MHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKVLTKCQEEVSHIPAVHPGSFRPKCDENGNYLPLQCYGSIGYCWCVFPNGTEVPNTRSRGHHNCSESLELEDPSSGLGVTKQDLGPVPM

SEQ ID NO: 134 - protein, 163 aa, Homo sapiens
  ref <308>: DATABASE ACCESSION NUMBER: Swissprot/Q99969
  ref <309>: DATABASE ENTRY DATE: 2000-05-30
  ref <313>: RELEVANT RESIDUES IN SEQ ID NO: (1)..(163)
MRRLLIPLALWLGAVGVGVAELTEAQRRGLQVALEEFHKHPPVQWAFQETSVESAVDTPFPAGIFVRLEFKLQQTSCRKRDWKKPECKVRPNGRKRKCLACIKLGSEDKVLGRLVHCPIETQVLREAEEHQETQCLRVQRAGEDPHSFYFPGQFAFSKALPRS

SEQ ID NO: 135 - protein, 2386 aa, Homo sapiens
  ref <308>: DATABASE ACCESSION NUMBER: Swissprot/P02751
  ref <309>: DATABASE ENTRY DATE: 1986-07-21
  ref <313>: RELEVANT RESIDUES IN SEQ ID NO: (1)..(2386)
MLRGPGPGLLLLAVQCLGTAVPSTGASKSKRQAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTQGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWNAPQPSHISKYILRWRPKNSVGRWKEATIPGHLNSYTIKGLKPGVVYEGQLISIQQYGHQEVTRFDFTTTSTSTPVTSNTVTGETTPFSPLVATSESVTEITASSFVVSWVSASDTVSGFRVEYELSEEGDEPQYLDLPSTATSVNIPDLLPGRKYIVNVYQISEDGEQSLILSTSQTTAPDAPPDPTVDQVDDTSIVVRWSRPQAPITGYRIVYSPSVEGSSTELNLPETANSVTLSDLQPGVQYNITIYAVEENQESTPVVIQQETTGTPRSDTVPSPRDLQFVEVTDVKVTIMWTPPESAVTGYRVDVIPVNLPGEHGQRLPISRNTFAEVTGLSPGVTYYFKVFAVSHGRESKPLTAQQTTKLDAPTNLQFVNETDSTVLVRWTPPRAQITGYRLTVGLTRRGQPRQYNVGPSVSKYPLRNLQPASEYTVSLVAIKGNQESPKATGVFTTLQPGSSIPPYNTEVTETTIVITWTPAPRIGFKLGVRPSQGGEAPREVTSDSGSIVVSGLTPGVEYVYTIQVLRDGQERDAPIVNKVVTPLSPPTNLHLEANPDTGVLTVSWERSTTPDITGYRITTTPTNGQQGNSLEEVVHADQSSCTFDNLSPGLEYNVSVYTVKDDKESVPISDTIIPAVPPPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQMQVTDVQDNSISVKWLPSSSPVTGYRVTTTPKNGPGPTKTKTAGPDQTEMTIEGLQPTVEYVVSVYAQNPSGESQPLVQTAVTNIDRPKGLAFTDVDVDSIKIAWESPQGQVSRYRVTYSSPEDGIHELFPAPDGEEDTAELQGLRPGSEYTVSVVALHDDMESQPLIGTQSTAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPSTVQKTPFVTHPGYDTGNGIQLPGTSGQQPSVGQQMIFEEHGFRRTTPPTTATPIRHRPRPYPPNVGEEIQIGHIPREDVDYHLYPHGPGLNPNASTGQEALSQTTISWAPFQDTSEYIISCHPVGTDEEPLQFRVPGTSTSATLTGLTRGATYNIIVEALKDQQRHKVREEVVTVGNSVNEGLNQPTDDSCFDPYTVSHYAVGDEWERMSESGFKLLCQCLGFGSGHFRCDSSRWCHDNGVNYKIGEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQADREDSRE

SEQ ID NO: 136 - protein, 339 aa, Homo sapiens [sequence omitted]

SEQ ID NO: 137 - protein, 1249 aa, Homo sapiens
  ref <308>: DATABASE ACCESSION NUMBER: Swissprot/P29144
  ref <309>: DATABASE ENTRY DATE: 1992-12-01
  ref <313>: RELEVANT RESIDUES IN SEQ ID NO: (1)..(1249)
MATAATEEPFPFHGLLPKKETGAASFLCRYPEYDGRGVLIAVLDTGVDPGAPGMQVTTDGKPKIVDIIDTTGSGDVNTATEVEPKDGEIVGLSGRVLKIPASWTNPSGKYHIGIKNGYDFYPKALKERIQKERKEKIWDPVHRVALAEACRKQEEFDVANNGSSQANKLIKEELQSQVELLNSFEKKYSDPGPVYDCLVWHDGEVWRACIDSNEDGDLSKSTVLRNYKEAQEYGSFGTAEMLNYSVNIYDDGNLLSIVTSGGAHGTHVASIAAGHFPEEPERNGVAPGAQILSIKIGDTRLSTMETGTGLIRAMIEVINHKCDLVNYSYGEATHWPNSGRICEVINEAVWKHNIIYVSSAGNNGPCLSTVGCPGGTTSSVIGVGAYVSPDMMVAEYSLREKLPANQYTWSSRGPSADGALGVSISAPGGAIASVPNWTLRGTQLMNGTSMSSPNACGGIALILSGLKANNIDYTVHSVRRALENTAVKADNIEVFAQGHGIIQVDKAYDYLVQNTSFANKLGFTVTVGNNRGIYLRDPVQVAAPSDHGVGIEPVFPENTENSEKISLQLHLALTSNSSWVQCPSHLELMNQCRHINIRVDPRGLREGLHYTEVCGYDIASPNAGPLFRVPITAVIAAKVNESSHYDLAFTDVHFKPGQIRRHFIEVPEGATWAEVTVCSCSSEVSAKFVLHAVQLVKQRAYRSHEFYKFCSLPEKGTLTEAFPVLGGKAIEFCIARWWASLSDVNIDYTISFHGIVCTAPQLNIHASEGINRFDVQSSLKYEDLAPCITLKNWVQTLRPVSAKTKPLGSRDVLPNNRQLYEMVLTYNFHQPKSGEVTPSCPLLCELLYESEFDSQLWIIFDQNKRQMGSGDAYPHQYSLKLEKGDYTIRLQIRHEQISDLERLKDLPFIVSHRLSNTLSLDIHENHSFALLGKKKSSNLTLPPKYNQPFFVTSLPDDKIPKGAGPGCYLAGSLTLSKTELGKKADVIPVHYYLIPPPTKTKNGSKDKEKDSEKEKDLKEEFTEALRDLKIQWMTKLDSSDIYNELKETYPNYLPLYVARLHQLDAEKERMKRLNEIVDAANAVISHIDQTALAVYIAMKTDPRPDAATIKNDMDKQKSTLVDALCRKGCALADHLLHTQAQDGAISTDAEGKEEEGESPLDSLAETFWETTKWTDLFDNKVLTFAYKHALVNKMYGRGLKFATKLVEEKPTKENWKNCIQLMKLLGWTHCASFTENWLPIMYPPDYCVF

SEQ ID NO: 138 - protein, 433 aa, Homo sapiens
  ref <308>: DATABASE ACCESSION NUMBER: Swissprot/Q99538
  ref <309>: DATABASE ENTRY DATE: 1997-11-01
  ref <313>: RELEVANT RESIDUES IN SEQ ID NO: (1)..(433)
MVWKVAVFLSVALGIGAVPIDDPEDGGKHWVVIVAGSNGWYNYRHQADACHAYQIIHRNGIPDEQIVVMMYDDIAYSEDNPTPGIVINRPNGTDVYQGVPKDYTGEDVTPQNFLAVLRGDAEAVKGIGSGKVLKSGPQDHVFIYFTDHGSTGILVFPNEDLHVKDLNETIHYMYKHKMYRKMVFYIEACESGSMMNHLPDNINVYATTAANPRESSYACYYDEKRSTYLGDWYSVNWMEDSDVEDLTKETLHKQYHLVKSHTNTSHVMQYGNKTISTMKVMQFQGMKRKASSPVPLPPVTHLDLTPSPDVPLTIMKRKLMNTNDLEESRQLTEEIQRHLDARHLIEKSVRKIVSLLAASEAEVEQLLSERAPLTGHSCYPEALLHFRTHCFNWHSPTYEYALRHLYVLVNLCEKPYPLHRIKLSMDHVCLGHY

SEQ ID NO: 139 - protein, 342 aa, Homo sapiens
  ref <308>: DATABASE ACCESSION NUMBER: Swissprot/P25105
  ref <309>: DATABASE ENTRY DATE: 1992-05-01
  ref <313>: RELEVANT RESIDUES IN SEQ ID NO: (1)..(342)
MEPHDSSHMDSEFRYTLFPIVYSIIFVLGVIANGYVLWVFARLYPCKKFNEIKIFMVNLTMADMLFLITLPLWIVYYQNQGNWILPKFLCNVAGCLFFINTYCSVAFLGVITYNRFQAVTRPIKTAQANTRKRGISLSLVIWVAIVGAASYFLILDSTNTVPDSAGSGNVTRCFEHYEKGSVPVLIIHIFIVFSFFLVFLIILFCNLVIIRTLLMQPVQQQRNAEVKRRALWMVCTVLAVFIICFVPHHVVQLPWTLAELGFQDSKFHQAINDAHQVTLCLLSTNCVLDPVIYCFLTKKFRKHLTEKFYSMRSSRKCSRATTDTVTEVVVPFNQIPGNSLKN

SEQ ID NO: 140 - protein, 359 aa, Homo sapiens
  ref <308>: DATABASE ACCESSION NUMBER: Swissprot/Q92187
  ref <309>: DATABASE ENTRY DATE: 1997-11-01
  ref <313>: RELEVANT RESIDUES IN SEQ ID NO: (1)..(359)
MRSIRKRWTICTISLLLIFYKTKEIARTEEHQETQLIGDGELSLSRSLVNSSDKIIRKAGSSIFQHNVEGWKINSSLVLEIRKNILRFLDAERDVSVVKSSFKPGDVIHYVLDRRRTLNISHDLHSLLPEVSPMKNRRFKTCAVVGNSGILLDSECGKEIDSHNFVIRCNLAPVVEFAADVGTKSDFITMNPSVVQRAFGGFRNESDREKFVHRLSMLNDSVLWIPAFMVKGGEKHVEWVNALILKNKLKVRTAYPSLRLIHAVRGYWLTNKVPIKRPSTGLLMYTLATRFCDEIHLYGFWPFPKDLNGKAVKYHYYDDLKYRYFSNASPHRMPLEFKTLNVLHNRGALKLTTGKCVKQ

SEQ ID NO: 141 - protein, 218 aa, Homo sapiens
  ref <308>: DATABASE ACCESSION NUMBER: Swissprot/Q15907
  ref <309>: DATABASE ENTRY DATE: 1998-07-15
  ref <313>: RELEVANT RESIDUES IN SEQ ID NO: (1)..(218)
MGTRDDEYDYLFKVVLIGDSGVGKSNLLSRFTRNEFNLESKSTIGVEFATRSIQVDGKTIKAQIWDTAGQERYRAITSAYYRGAVGALLVYDIAKHLTYENVERWLKELRDHADSNIVIMLVGNKSDLRHLRAVPTDEARAFAEKNNLSFIETSALDSTNVEEAFKNILTEIYRIVSQKQIADRAAHDESPGNNVVDISVPPTTDGQKPNKLQCCQNL